(19) United States
(12) Patent Application Publication
(10) Pub. No.: US 2025/0109143 A1
(43) Pub. Date: Apr. 3, 2025

(54) KLF2 INDUCING DEUTERATED COMPOUNDS AND USES THEREOF

(71) Applicants: Riparian Pharmaceuticals, Inc., Watertown, MA (US); Pfizer Inc., New York, NY (US)

(72) Inventors: Michael Serrano-Wu, Boston, MA (US); Jamison Bryce Tuttle, Marblehead, MA (US); Ronald T. Wester, Ledyard, CT (US)

(73) Assignees: Riparian Pharmaceuticals, Inc., Watertown, MA (US); Pfizer Inc., New York, NY (US)

(21) Appl. No.: 18/792,742

(22) Filed: Aug. 2, 2024

Related U.S. Application Data
(60) Provisional application No. 63/517,630, filed on Aug. 4, 2023.

(51) Int. Cl. C07D 498/04; A61K 31/553; C07B 59/00

(52) U.S. Cl. CPC C07D 498/04; A61K 31/553; C07B 59/002

(57) ABSTRACT

The present disclosure provides deuterated compounds that are inducers of KLF2 and pharmaceutical compositions comprising the same. The present disclosure further provides method of treating an inflammatory disease or endothelial dysfunction comprising administering a therapeutically effective amount of the deuterated compounds disclosed herein.

### STATEMENT OF GOVERNMENT SUPPORT

This invention was made with government support under Grant Number R44HL118826 awarded by the National Institutes of Health National Heart, Lung and Blood Institute. The government has certain rights in the invention.

### BACKGROUND

The vascular endothelium is an important regulator of vascular integrity and vascular homeostasis. The vascular endothelium is a dynamic interface that regulates vasotone, inflammation, hemostasis and vascular remodeling. Dysfunction of the vascular endothelium, including vasoconstriction, impaired vasoreactivity, inflammation, thrombosis, altered barrier permeability and loss of vascular quiescence, is a key driver of many vascular diseases. As such, the vascular endothelium is important for maintaining vascular and cardiovascular health.

Krüppel-like Factor 2 (KLF2) is a shear stress-induced transcription factor that may confer anti-inflammatory and/or anti-thrombotic properties to vascular endothelial cells. In endothelial cells, KLF2 may be involved in transcriptional processes for regulating inflammation, thrombosis hemostasis, vascular tone, and blood vessel development. KLF2 is a key regulator of activation, differentiation, and migration processes in various immune cell types including monocytes, macrophages, neutrophils, T lymphocytes, B lymphocytes and natural killer cells.

Accordingly, compounds that induce KLF2 may be useful for maintaining vascular health or for treating vascular or inflammatory conditions.

### SUMMARY OF THE INVENTION

The present disclosure provides, in part, compounds of Formula I, Ia, Ib, Ic, Id, or Ie and pharmaceutically acceptable salts thereof. Such compounds may modulate the activity of KLF2 and may be useful in the treatment of an inflammatory disorder or endothelial dysfunction. Also provided are pharmaceutical compositions, comprising the compounds or salts of the disclosure, alone or in combination with additional therapeutic agents such as cardiovascular drugs. The present disclosure also provides, in part, methods for preparing such compounds, pharmaceutically acceptable salts and compositions of the disclosure, and methods of using the foregoing. This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

According to an embodiment of the disclosure there is provided a compound of Formula Ia:

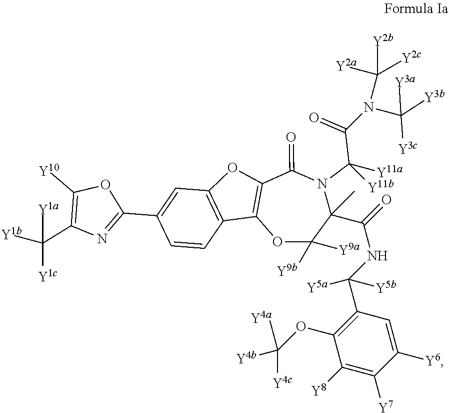

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H or D.

In one embodiment, the disclosure provides a compound of Formula Ib:

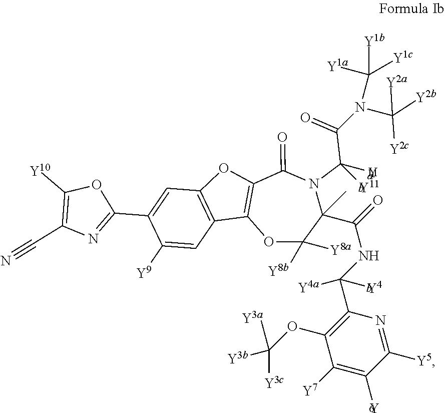

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

In one embodiment, the disclosure provides a compound of Formula Ic:

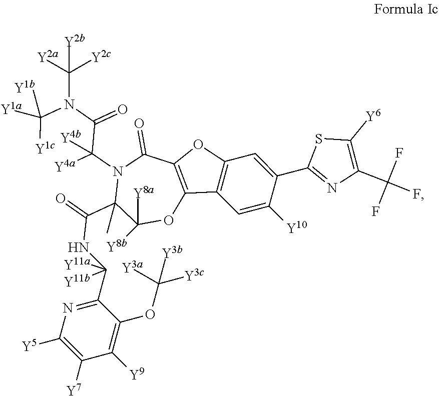

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

In one embodiment, the disclosure provides a compound of Formula Id:

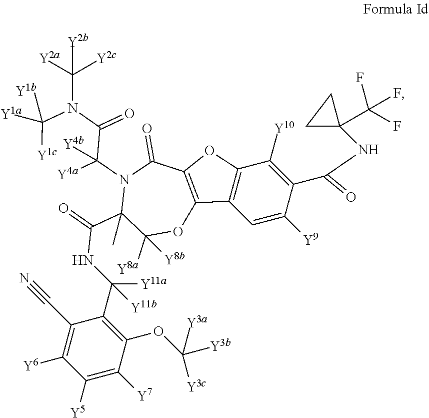

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

In one embodiment, the disclosure provides a compound of Formula Ie:

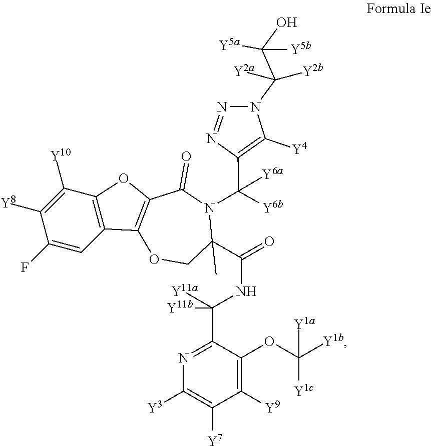

or a pharmaceutically acceptable salt thereof, wherein each Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H or D.

In one embodiment, disclosed herein is a pharmaceutical composition In one embodiment, the disclosure provides a compound of Formula Ib: comprising a compound of Formula I, Ia, Ib, Ic, Id, or Ie, or a pharmaceutically acceptable salt thereof; and a pharmaceutically acceptable carrier.

In one embodiment, disclosed herein is a method of treating a condition comprising administering a therapeutically effective amount of a compound of Formula I, Ia, Ib, Ic, Id, or Ie, or a pharmaceutically acceptable salt thereof, to a subject in need thereof, wherein the condition is an inflammatory disease or endothelial dysfunction.

It is to be understood that both the foregoing general description and the following detailed description are exemplary and explanatory only and are not restrictive of the disclosure, as claimed.

### DETAILED DESCRIPTION

The present disclosure may be understood more readily by reference to the following detailed description of the embodiments of the disclosure and the Examples included herein. It is to be understood that this disclosure is not limited to specific synthetic methods of making that may of course vary. It is to be also understood that the terminology used herein is for the purpose of describing specific embodiments only and is not intended to be limiting.

### Definitions

Unless otherwise defined herein, scientific and technical terms used in connection with the present disclosure have the meanings that are commonly understood by those of ordinary skill in the art. The disclosure described herein suitably may be practiced in the absence of any element(s) not specifically disclosed herein.

“Compounds of the disclosure” include compounds of Formula I, Ia, Ib, Ic, Id, or Ie, or a pharmaceutically acceptable salt thereof, and the novel intermediates used in the preparation thereof. One of ordinary skill in the art will appreciate that compounds of the disclosure include conformational isomers (e.g., cis and trans isomers) and all optical isomers (e.g., enantiomers and diastereomers), racemic, diastereomeric and other mixtures of such isomers, tautomers thereof, where they may exist. One of ordinary skill in the art will also appreciate that compounds of the disclosure include solvates, hydrates, isomorphs, polymorphs, esters, salt forms, prodrugs, and isotopically labelled versions thereof (including deuterium substitutions), where they may be formed.

As used herein, the singular form “a”, “an”, and “the” include plural references unless indicated otherwise. For example, “a” substituent includes one or more substituents.

As used herein, the term “about” when used to modify a numerically defined parameter (e.g., the dose of a compound) means that the parameter may vary by as much as 10% below or above the stated numerical value for that parameter. For example, a dose of about 5 mg means 5%±10%, i.e., it may vary between 4.5 mg and 5.5 mg.

If substituents are described as being “independently selected” from a group, each substituent is selected independent of the other. Each substituent therefore may be identical to or different from the other substituent(s).

“Optional” or “optionally” means that the subsequently described event or circumstance may, but need not occur, and the description includes instances where the event or circumstance occurs and instances in which it does not.

The terms “optionally substituted” and “substituted or unsubstituted” are used interchangeably to indicate that the particular group being described may have no non-hydrogen substituents (i.e., unsubstituted), or the group may have one or more non-hydrogen substituents (i.e., substituted). If not otherwise specified, the total number of substituents that may be present is equal to the number of H atoms present on the unsubstituted form of the group being described. Where an optional substituent is attached via a double bond, such as an oxo (═O) substituent, the group occupies two available valences, so the total number of other substituents that are included is reduced by two. In the case where optional substituents are selected independently from a list of alternatives, the selected groups may be the same or different. Throughout the disclosure, it will be understood that the number and nature of optional substituent groups will be limited to the extent that such substitutions make chemical sense to one of ordinary skill in the art.

“Halogen” or “halo” refers to fluoro, chloro, bromo and iodo (F, Cl, Br, I).

“Cyano” refers to a substituent having a carbon atom joined to a nitrogen atom by a triple bond, i.e., —C≡N.

“Hydroxy” refers to an —OH group.

“Oxo” refers to a double bonded oxygen (═O).

“Alkyl” refers to a saturated, monovalent aliphatic hydrocarbon radical that has a specified number of carbon atoms, including straight chain or branched chain groups. Alkyl groups may contain, but are not limited to, 1 to 12 carbon atoms (“C₁-C₁₂ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄ alkyl”), 1 to 3 carbon atoms (“C₁-C₃ alkyl”), or 1 to 2 carbon atoms (“C₁-C₂ alkyl”). Examples include, but are not limited to, methyl, ethyl, n-propyl, isopropyl, n-butyl, sec-butyl, isobutyl, tert-butyl, n-pentyl, isopentyl, neopentyl, n-hexyl, n-heptyl, n-octyl, and the like. Alkyl groups may be optionally substituted, unsubstituted or substituted, as further defined herein. In some instances, substituted alkyl groups are specifically named by reference to the substituent group. For example, “haloalkyl” refers to an alkyl group having the specified number of carbon atoms that is substituted by one or more halo substituents, up to the available valence number.

“Haloalkyl” refers to an alkyl group as defined above containing the specified number of carbon atoms wherein at least one hydrogen atom has been replaced by halogen. Haloalkyl groups man contain, but are not limited to, 1-6 carbon atoms (“C₁-C₆ haloalkyl”), 1-4 carbon atoms (“C₁-C₄ haloalkyl”), or 1-2 carbon atoms (“C₁-C₂ haloalkyl”). More specifically, fluorinated alkyl groups may be specifically referred to as “fluoroalkyl.”

“Fluoroalkyl” refers to an alkyl group, as defined herein, wherein from one to all of the hydrogen atoms of the alkyl group are replaced by fluoro atoms. Examples include, but are not limited to, fluoromethyl, difluoromethyl, fluoroethyl, difluoroethyl, trifluoroethyl, and tetrafluoroethyl. Examples of fully substituted fluoroalkyl groups (also referred to as perfluoroalkyl groups) include trifluoromethyl (—CF₃) and pentafluoroethyl (—C₂F₅).

“Alkoxy” refers to an alkyl group, as defined herein, that is single bonded to an oxygen atom. The attachment point of an alkoxy radical to a molecule is through the oxygen atom. An alkoxy radical may be depicted as alkyl-O—. Alkoxy groups may contain, but are not limited to, 1 to 8 carbon atoms (“C₁-C₈ alkoxy”), 1 to 6 carbon atoms (“C₁-C₆ alkoxy”), 1 to 4 carbon atoms (“C₁-C₄ alkoxy”), or 1 to 3 carbon atoms (“C₁-C₃ alkoxy”). Alkoxy groups include, but are not limited to, methoxy, ethoxy, n-propoxy, isobutoxy, and the like.

“Haloalkoxy” refers to an alkoxyl group as defined above containing the specified number of carbon atoms wherein at least one hydrogen atom has been replaced by halogen. Haloalkoxy groups may contain, but are not limited to, 1-6 carbon atoms, (“C₁-C₆ haloalkoxy”), 1-4 carbon atoms (“C₁-C₄ haloalkoxy”), or 1-2 carbon atoms (“C₁-C₂ haloalkoxy”). More specifically, fluorinated alkoxyl groups may be specifically referred to as “fluoroalkoxy.”

“Alkoxyalkyl” refers to an alkyl group, as defined herein, that is substituted by an alkoxy group, as defined herein. Examples include, but are not limited to, CH₃OCH₂— and CH₃CH₂OCH₂—.

“Alkenyl” refers to an alkyl group, as defined herein, consisting of at least two carbon atoms and at least one carbon-carbon double bond. For example, as used herein, the term “C₂-C₆ alkenyl” means straight or branched chain unsaturated radicals of 2 to 6 carbon atoms, including, but not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Examples include, but are not limited to, ethynyl, 1-propynyl, 2-propynyl, 1-, 2-, or 3-butynyl, and the like.

“Cycloalkyl” refers to a fully saturated hydrocarbon ring system that has the specified number of carbon atoms, which may be a monocyclic, bridged or fused bicyclic or polycyclic ring system that is connected to the base molecule through a carbon atom of the cycloalkyl ring. Cycloalkyl groups may contain, but are not limited to, 3 to 12 carbon atoms (“C₃-C₁₂ cycloalkyl”), 3 to 8 carbon atoms (“C₃-C₈ cycloalkyl”), 3 to 6 carbon atoms (“C₃-C₆ cycloalkyl”), 3 to 5 carbon atoms (“C₃-C₅ cycloalkyl”) or 3 to 4 carbon atoms (“C₃-C₄ cycloalkyl”). Examples include, but are not limited to, cyclopropyl, cyclobutyl, cyclopentyl, cyclohexyl, cycloheptyl, adamantanyl, and the like. Cycloalkyl groups may be optionally substituted, unsubstituted or substituted, as further defined herein.

“Cycloalkoxy” refers to a cycloalkyl group, as defined herein, that is single bonded to an oxygen atom. The attachment point of a cycloalkoxy radical to a molecule is through the oxygen atom. A cycloalkoxy radical may be depicted as cycloalkyl-O—. Cycloalkoxy groups may contain, but are not limited to, 3 to 8 carbon atoms (“C₃-C₈ cycloalkoxy”), 3 to 6 carbon atoms (“C₃-C₆ cycloalkoxy”), and 3 to 4 carbon atoms (“C₃-C₄ cycloalkoxy”). Representative cycloalkyl rings include cyclopropyl, cyclobutyl, cyclopentyl, cyclohexyl, cycloheptyl, and cyclooctyl. Polycyclic cycloalkyl groups include, for example, adamantanyl, 1,2-dihydronaphthalenyl, 1,4-dihydronaphthalenyl, tetraenyl, decalinyl, 3,4-dihydronaphthalenyl-1(2H)-one, spiro[2.2]pentyl, norbornyl, and bicycle[1.1.1]pentyl.

Heterocycloalkyl” refers to a fully saturated ring system containing the specified number of ring atoms and containing at least one heteroatom selected from N, O and S as a ring member, where ring S atoms are optionally substituted by one or two oxo groups (i.e., S(O)_{q}, where q is 0, 1 or 2) and where the heterocycloalkyl ring is connected to the base molecule via a ring atom, which may be C or N. Heterocycloalkyl rings include rings which are spirocyclic, bridged, or fused to one or more other heterocycloalkyl or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocycloalkyl portion of the ring system. Heterocycloalkyl rings may contain 1 to 4 heteroatoms selected from N, O, and S(O)_{q} as ring members, or 1 to 2 ring heteroatoms, provided that such heterocycloalkyl rings do not contain two contiguous oxygen or sulfur atoms.

Heterocycloalkyl rings may be optionally substituted, unsubstituted or substituted, as further defined herein. Such substituents may be present on the heterocyclic ring attached to the base molecule, or on a spirocyclic, bridged or fused ring attached thereto.

Heterocycloalkyl rings may include, but are not limited to, 3-8 membered heterocyclyl groups, for example 4-7 or 4-6 membered heterocycloalkyl groups, in accordance with the definition herein. Illustrative examples of heterocycloalkyl rings include, but are not limited to a monovalent radical of oxirane (oxiranyl), thiirane (thiiranyl), aziridine (aziridinyl), oxetane (oxetanyl), thietane (thietanyl), azetidine (azetidinyl), tetrahydrofuran (tetrahydrofuranyl), tetrahydrothiophene (tetrahydrothiophenyl), pyrrolidine (pyrrolidinyl), tetrahydropyran (tetrahydropyranyl), tetrahydrothiopyran (tetrahydrothiopyranyl), piperidine (piperidinyl), 1,4-dioxane (1,4-dioxanyl), 1,4-oxathiarane (1,4-oxathiaranyl), morpholine (morpholinyl), 1,4-dithiane (1,4-dithianyl), piperazine (piperazinyl), thiomorpholine (thiomorpholinyl), oxepane (oxepanyl), thiepane (thiepanyl), azepane (azepanyl), 1,4-dioxepane (1,4-dioxepanyl), 1,4-oxathiepane (1,4-oxathiepanyl), 1,4-oxaazepane (1,4-oxaazepanyl), 1,4-thiazepane (1,4-thiazapanyl), 1,4-diazepane (1,4-diazepanyl), or 1,4-dithepane (1,4-dithiepanyl). Illustrative examples of bridged and fused heterocycloalkyl groups include, but are not limited to a monovalent radical of 1-oxa-5-azabicyclo-[2.2.1]heptane, 3-oxa-8-azabicyclo-[3.2.1]octane, 3-azabicyclo-[3.1.0]hexane, or 2-azabicyclo-[3.1.0]hexane.

“Aryl” or “aromatic” refers to monocyclic, bicyclic (e.g., biaryl, fused) or polycyclic ring systems that contain the specified number of ring atoms, in which all carbon atoms in the ring are of sp² hybridization and in which the pi electrons are in conjugation. Aryl groups may contain, but are not limited to, 6 to 20 carbon atoms (“C₆-C₂₀ aryl”), 6 to 14 carbon atoms (“C₆-C₁₄ aryl”), 6 to 12 carbon atoms (“C₆-C₁₂ aryl”), or 6 to 10 carbon atoms (“C₆-C₁₀ aryl”). Fused aryl groups may include an aryl ring (e.g., a phenyl ring) fused to another aryl ring. Examples include, but are not limited to, phenyl, biphenyl, naphthyl, anthracenyl, phenanthrenyl, indanyl, and indenyl. Aryl groups may be optionally substituted, unsubstituted or substituted, as further defined herein.

Similarly, “heteroaryl” or “heteroaromatic” refer to monocyclic, bicyclic (e.g., heterobiaryl, fused) or polycyclic ring systems that contain the specified number of ring atoms and include at least one heteroatom selected from N, O and S as a ring member in a ring in which all carbon atoms in the ring are of sp² hybridization and in which the pi electrons are in conjugation. Heteroaryl groups may contain, but are not limited to, 5 to 20 ring atoms (“5-20 membered heteroaryl”), 5 to 14 ring atoms (“5-14 membered heteroaryl”), 5 to 12 ring atoms (“5-12 membered heteroaryl”), 5 to 10 ring atoms (“5-10 membered heteroaryl”), 5 to 9 ring atoms (“5-9 membered heteroaryl”), or 5 to 6 ring atoms (“5-6 membered heteroaryl”). Heteroaryl rings are attached to the base molecule via a ring atom of the heteroaromatic ring. Thus, either 5- or 6-membered heteroaryl rings, alone or in a fused structure, may be attached to the base molecule via a ring C or N atom. Examples of heteroaryl groups include, but are not limited to, pyrrolyl, furanyl, thiophenyl, pyrazolyl, imidazolyl, isoxazolyl, oxazolyl, isothiazolyl, thiazolyl, triazolyl, oxadiazolyl, thiadiazolyl, tetrazolyl, pyridinyl, pyridizinyl, pyrimidinyl, pyrazinyl, benzofuranyl, benzothiophenyl, indolyl, benzimidazolyl, indazolyl, quinolinyl, isoquinolinyl, purinyl, triazinyl, naphthyridinyl, cinnolinyl, quinazolinyl, quinoxalinyl and carbazolyl. Examples of 5- or 6-membered heteroaryl groups include, but are not limited to, pyrrolyl, furanyl, thiophenyl, pyrazolyl, imidazolyl, isoxazolyl, oxazolyl, isothiazolyl, thiazolyl, triazolyl, pyridinyl, pyrimidinyl, pyrazinyl and pyridazinyl rings. Heteroaryl groups may be optionally substituted, unsubstituted or substituted, as further defined herein.

Illustrative examples of monocyclic heteroaryl groups include, but are not limited to a monovalent radical of pyrrole (pyrrolyl), furan (furanyl), thiophene (thiophenyl), pyrazole (pyrazolyl), imidazole (imidazolyl), isoxazole (isoxazolyl), oxazole (oxazolyl), isothiazole (isothiazolyl), thiazolyl (thiazolyl), 1,2,3-triazole (1,2,3-triazolyl), 1,3,4-triazole (1,3,4-triazolyl), 1-oxa-2,3-diazole (1-oxa-2,3-diazolyl), 1-oxa-2,4-diazole (1-oxa-2,4-diazolyl), 1-oxa-2,5-diazole (1-oxa-2,5-diazolyl), 1-oxa-3,4-diazole (1-oxa-3,4-diazolyl), 1-thia-2,3-diazole (1-thia-2,3-diazolyl), 1-thia-2,4-diazole (1-thia-2,4-diazolyl), 1-thia-2,5-diazole (1-thia-2,5-diazolyl), 1-thia-3,4-diazole (1-thia-3,4-diazolyl), tetrazole (tetrazolyl), pyridine (pyridinyl), pyridazine (pyridazinyl), pyrimidine (pyrimidinyl), or pyrazine (pyrazinyl).

Illustrative examples of fused ring heteroaryl groups include, but are not limited to benzofuran (benzofuranyl), benzothiophene (benzothiophenyl), indole (indolyl), benzimidazole (benzimidazolyl), indazole (indazolyl), benzotriazole (benzotriazolyl), pyrrolo[2,3-b]pyridine (pyrrolo[2,3-b]pyridinyl), pyrrolo[2,3-c]pyridine (pyrrolo[2,3-c]pyridinyl), pyrrolo[3,2-c]pyridine (pyrrolo[3,2-c]pyridinyl), pyrrolo[3,2-b]pyridine (pyrrolo[3,2-b]pyridinyl), imidazo[4,5-b]pyridine (imidazo[4,5-b]pyridinyl), imidazo[4,5-c]pyridine (imidazo[4,5-c]pyridinyl), pyrazolo[4,3-d]pyridine (pyrazolo[4,3-d]pyridinyl), pyrazolo[4,3-c]pyridine (pyrazolo[4,3-c]pyridinyl), pyrazolo[3,4-c]pyridine (pyrazolo[3,4-c]pyridinyl), pyrazolo[3,4-b]pyridine (pyrazolo[3,4-b]pyridinyl), isoindole (isoindolyl), indazole (indazolyl), purine (purinyl), indolizine (indolizinyl), imidazo[1,2-a]pyridine (imidazo[1,2-a]pyridinyl), imidazo[1,5-a]pyridine (imidazo[1,5-a]pyridinyl), pyrazolo[1,5-a]pyridine (pyrazolo[1,5-a]pyridinyl), pyrrolo[1,2-b]pyridazine (pyrrolo[1,2-b]pyridazinyl), imidazo[1,2-c]pyrimidine (imidazo[1,2-c]pyrimidinyl), quinoline (quinolinyl), isoquinoline (isoquinolinyl), cinnoline (cinnolinyl), quinazoline (azaquinazoline), quinoxaline (quinoxalinyl), phthalazine (phthalazinyl), 1,6-naphthyridine (1,6-naphthyridinyl), 1,7-naphthyridine (1,7-naphthyridinyl), 1,8-naphthyridine (1,8-naphthyridinyl), 1,5-naphthyridine (1,5-naphthyridinyl), 2,6-naphthyridine (2,6-naphthyridinyl), 2,7-naphthyridine (2,7-naphthyridinyl), pyrido[3,2-d]pyrimidine (pyrido[3,2-d]pyrimidinyl), pyrido[4,3-d]pyrimidine (pyrido[4,3-d]pyrimidinyl), pyrido[3,4-d]pyrimidine (pyrido[3,4-d]pyrimidinyl), pyrido[2,3-d]pyrimidine (pyrido[2,3-d]pyrimidinyl), pyrido[2,3-b]pyrazine (pyrido[2,3-b]pyrazinyl), pyrido[3,4-b]pyrazine (pyrido[3,4-b]pyrazinyl), pyrimido[5,4-d]pyrimidine (pyrimido[5,4-d]pyrimidinyl), pyrazino[2,3-b]pyrazine (pyrazino[2,3-b]pyrazinyl), or pyrimido[4,5-d]pyrimidine (pyrimido[4,5-d]pyrimidinyl).

“Amino” refers to a group —NH₂, which is unsubstituted. Where the amino is described as substituted or optionally substituted, the term includes groups of the form —NR′R″, where each of R′ and R″ is defined as further described herein. For example, “alkylamino” refers to a group —NRxRy, wherein one of Rx and Ry is an alkyl moiety and the other is H, and “dialkylamino” refers to —NRxRy wherein both of Rx and Ry are alkyl moieties, where the alkyl moieties have the specified number of carbon atoms (e.g., —NH(C₁-C₄ alkyl) or —N(C₁-C₄ alkyl)₂).

“Aminoalkyl” refers to an alkyl group, as defined above, that is substituted by 1, 2, or 3 amino groups, as defined herein.

The term “pharmaceutically acceptable” means the substance (e.g., the compounds described herein) and any salt thereof, or composition containing the substance or salt of the disclosure is suitable for administration to a subject or patient.

A “pharmaceutical composition” refers to a mixture of one or more of the compounds of the disclosure, or a pharmaceutically acceptable salt, solvate, hydrate or prodrug thereof as an active ingredient, and at least one pharmaceutically acceptable excipient.

“Deuterium enrichment factor” as used herein means the ratio between the deuterium abundance and the natural abundance of deuterium, each relative to hydrogen abundance. An atomic position designated as having deuterium typically has a deuterium enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“Excipient” as used herein describes any ingredient other than the compound(s) of the disclosure. The choice of excipient will to a large extent depend on factors such as the mode of administration, the effect of the excipient on solubility and stability, and the nature of the dosage form.

As used herein, “excipient” includes any and all solvents, dispersion media, coatings, antibacterial and antifungal agents, isotonic and absorption delaying agents, carriers, diluents and the like that are physiologically compatible. Examples of excipients include one or more of water, saline, phosphate buffered saline, dextrose, glycerol, ethanol and the like, as well as combinations thereof, and may include isotonic agents, for example, sugar, sodium chloride, or polyalcohol such as mannitol, or sorbitol in the composition. Examples of excipients also include various organic solvents (such as hydrates and solvates). The pharmaceutical compositions may, if desired, contain additional excipients such as flavorings, binders/binding agents, lubricating agents, disintegrants, sweetening or flavoring agents, coloring matters or dyes, and the like. For example, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of excipients, therefore, also include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with additional excipients such as water, ethanol, propylene glycol, glycerin, or combinations thereof.

Examples of excipients also include pharmaceutically acceptable substances such as wetting agents or minor amounts of auxiliary substances such as wetting or emulsifying agents, preservatives, or buffers, which enhance the shelf life or effectiveness of the compound.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

As used herein, the term, “subject, “individual” or “patient,” used interchangeably, refers to any animal, including mammals. Mammals according to the disclosure include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting (or slowing) further development of the pathology or symptomatology or both); and
(3) ameliorating the disease; for example, ameliorating a disease, condition or disorder in an individual that is experiencing or displaying the pathology or symptomatology of the disease, condition or disorder (i.e., reversing the pathology or symptomatology or both).

As used herein, the term “selective” describes a functionally-defined receptor ligand or enzyme inhibitor means selective for the defined receptor or enzyme subtype as compared with other receptor or enzyme subtypes in the same family. For instance, a selective beta blocker is a compound which inhibits one beta receptor more potently than another beta receptor. Such selectivity is, in one embodiment, at least 2 fold (as measured using conventional binding assays), or, in another embodiment, at least 10 fold, or, in a further embodiment, at least 100 fold.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to induce KLF2 and/or vaso-protection may render them suitable as “therapeutic agents” in the methods and compositions of this disclosure.

### Compounds of the Invention

In some embodiments, the invention provides a compound of formula I:

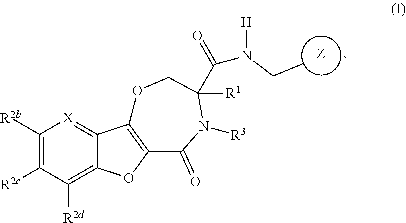

or a pharmaceutically acceptable salt thereof, wherein:
   R¹ is C₁₋₃alkyl;
   X is C—R^{2a} or N;
   R^{2a}, R^{2b}, R^{2c}, and R^{2d} each independently represent H, D, C₁₋₆alkyl, C₁₋₆alkyl-d₁₋₁₃, C₂₋₆alkenyl, C₂₋₆alkynyl, halo, aryl, heteroaryl, cycloalkyl, heterocyclyl, cyano, acyl, carboxy, ester, or amido; or R^{2a} and R^{2b} with the carbon atoms to which R^{2a} and R^{2b} are bound form a ring; R^{2b} and R^{2c} with the carbon atoms to which R^{2b} and R^{2c} are bound form a ring; or R^{2c} and R^{2d} with the carbon atoms to which R^{2c} and R^{2d} are bound form a ring; each of which is independently substituted or unsubstituted;
   R³ is C₁₋₆alkyl, C₁₋₆alkyl-d₁₋₁₃, C₂₋₆alkenyl, C₂₋₆alkynyl, aryl, heteroaryl, cycloalkyl, heterocyclyl, arylalkyl, heteroaralkyl, (cycloalkyl)alkyl, heterocyclylalkyl, amidoalkyl, alkoxyalkyl, or acylalkyl, each of which is independently unsubstituted or substituted; and
   Z is a substituted or unsubstituted aryl or heteroaryl, wherein the aryl or heteroaryl is each independently unsubstituted or substituted with one or more groups chosen from C₁₋₆alkyl, C₁₋₆alkyl-d₁₋₁₃, C₂₋₆alkenyl, C₂₋₆alkynyl, cyano, acyl, carboxy, ester, amido, alkoxy, or halo,
   wherein the compound comprises a D in at least one position, and wherein the level of deuterium at the at least one position is at least about 50%.

In certain embodiments, R¹ is methyl.

In certain embodiments, X is N. In other embodiments, X is C—R^{2a}.

In certain embodiments, R^{2a}, R^{2b}, R^{2c}, and R^{2d} independently represent hydrogen, methyl, propenyl, chloro, fluoro, haloalkyl (e.g., trifluoromethyl), a five-membered heteroaryl, cyclopropyl, or amido having the structure:

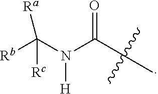

In certain such embodiments, R^{a} is H, D, C₁₋₆alkyl, or C₁₋₆alkyl-d₁₋₁₃, and R^{b} and R^{c} taken together form C₃₋₈cycloalkyl or heterocyclyl, e.g., cyclobutyl. In certain embodiments, R^{a} is H, D, methyl, or CD₃. In one embodiment, R^{a} is methyl or CD₃. In certain embodiments, R^{b} and R^{c} taken together form oxetane.

In certain embodiments, at least one of R^{2a}, R^{2b}, R^{2c}, and R^{2d} is the 5-membered heteroaryl. In some embodiments, the 5-membered heteroaryl is thiazolyl or oxazolyl, each of which is optionally substituted with D, trifluoromethyl, chloro, or cyano. In certain such embodiments, the 5-membered heteroaryl is oxazol-2-yl, e.g., 4-cyanooxazol-2-yl.

In certain embodiments, R^{2a} is H or D. In certain embodiments, R^{2b} and R^{2d} are each independently H or D. In certain embodiments, R^{2c} and R^{2d} are each independently H or D. In certain embodiments, R^{2b}, R^{2c}, and R^{2d} are each independently H or D.

In certain preferred embodiments, if X is C—R^{2a}, at least one of R^{2a}, R^{2b}, R^{2c}, and R^{2d} is not hydrogen, such that the benzofuran ring has at least one non-hydrogen substitution. For example in certain embodiments, if X is C—H, then preferably one of R^{2b}, R^{2c}, and R^{2d} is not hydrogen. In certain of these preferred embodiments, R^{2c} is the non-hydrogen substitution and is preferably a 5-membered heteroaryl such as oxazolyl, which is optionally substituted.

In certain embodiments, if X is C—R^{2a}, then R^{2a}, R^{2b}, R^{2c}, and R^{2d} are each H or D.

In certain preferred embodiments, R³ is amidoalkyl, such as an amidoalkyl having the structure:

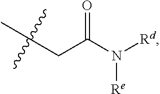

wherein R^{d} and R^{e} are independently chosen from alkyl or hydroxyalkyl, preferably alkyl (e.g., methyl), or R^{d} and R^{e} taken together form a heterocyclic ring. In certain such embodiments, R^{d} and R^{e} are each methyl. In certain embodiments, R^{d} and R^{e} are independently enriched for deuterium at one or more hydrogen-bearing sites. For example, R^{d} and/or R^{e} may contain a non-natural abundance of deuterium, preferably wherein a hydrogen position is at least about 15%, at least about 25%, at least about 50%, at least about 60%, at least about 75%, at least 80%, at least about 90%, at least about 98%, or at least about 99.5% deuterium. In certain embodiments, R^{d} is methyl or CD₃ and R^{e} is —(CH₂)₂OH.

In certain embodiments, R^{d} and R^{e} taken together with the nitrogen to which they are attached form an azetidine optionally substituted with one or more D, halo, hydroxyl, or hydroxyalkyl, for example:

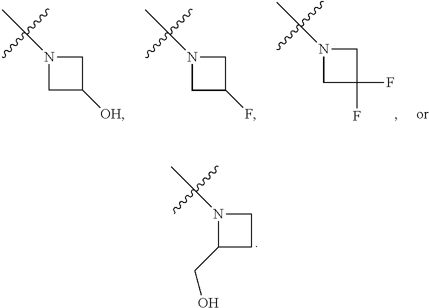 [SMILES: *N1CC(O)C1 ; *N1CC(F)C1 ; *N1CC(F)(F)C1 ; *N1CCC1CO]

In certain embodiments, R³ is C₃-C₆ cycloalkyl, for example:

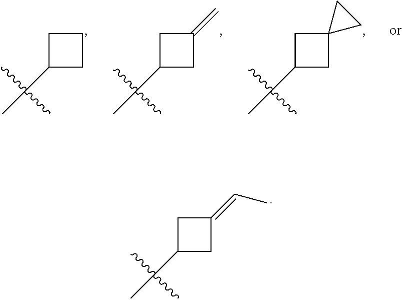

In certain embodiments, R³ is C₁-C₆ alkyl (including, for example, C₁₋₆alkyl, C₁₋₆alkyl-d₁₋₁₃, C₂-C₆alkenyl, or C₂₋₆ alkynyl, optionally substituted with alkoxy. In some embodiments, R³ is

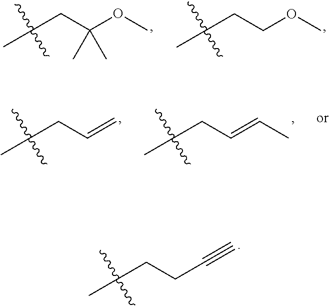

In certain embodiments, R³ is —CH₂-cycloalkyl optionally substituted with D, halo, alkoxy, or hydroxyl. In some embodiments, R³ is

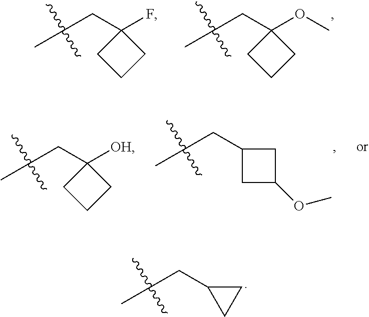

In certain embodiments, R³ is acylalkyl having the structure:

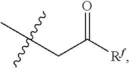

wherein R^{f} represents C₁₋₆alkyl, C₁₋₆alkyl-d₁₋₁₃, or C₃₋₈cycloalkyl. In some embodiments, R^{f} is ethyl or cyclopropyl, each of which is independently unsubstituted or substituted with D.

In certain embodiments, R³ represents —(CH₂)₁₋₃-heteroaryl, optionally substituted with D, C₁₋₆alkyl, C₁₋₆alkyl-d₁₋₁₃, C₁₋₆hydroxyalkyl or C₁₋₆alkoxyC₁₋₆alkoxyC₁₋₆alkyl. In certain embodiments, the heteroaryl is tetrazole, 1,2,3-triazole, or 1,2,4-triazole. In certain embodiments, R³ is:

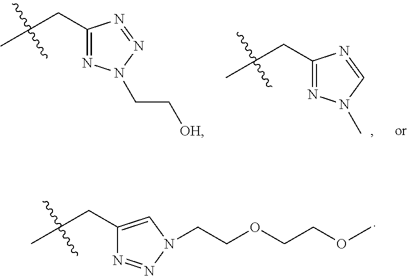

In certain embodiments, Z is phenyl, pyridinyl, naphthyl, isoquinolinyl, or quinolinyl, preferably pyridyl, each of which is optionally substituted with one or more groups chosen from D, C₁₋₃alkyl, C₁₋₃alkyl-d₁₋₁₃, C₁₋₃alkoxy, halo, C₁₋₃haloalkoxy, amido, and cyano. In certain embodiments, Z is substituted with one or more groups, or for example at least two groups, chosen from D, methoxy, isopropyloxy, chloro, fluoro, trifluoromethoxy, cyano, and carbamoyl. In certain embodiments, Z is phenyl substituted with methoxy and at least one additional substituent. In certain preferred embodiments, Z is pyridyl optionally substituted with an alkoxy (e.g., methoxy). In certain embodiments, Z is mono-, di-, or trisubstituted. In certain embodiments, Z is:

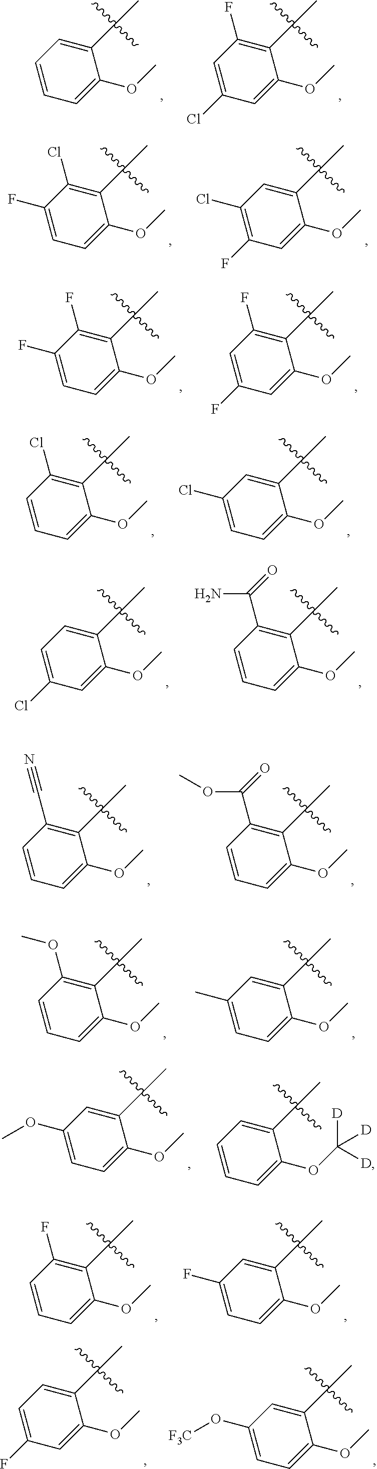

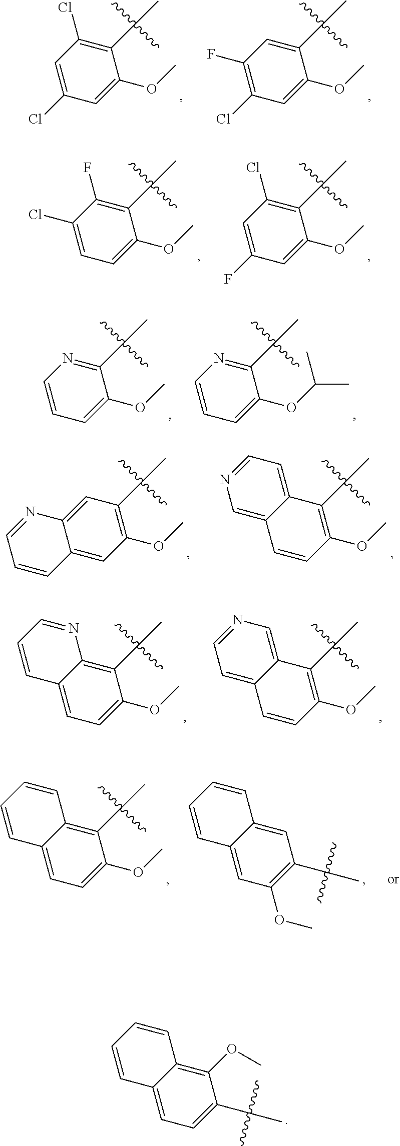

In certain embodiments, the compound of formula (I) is not:

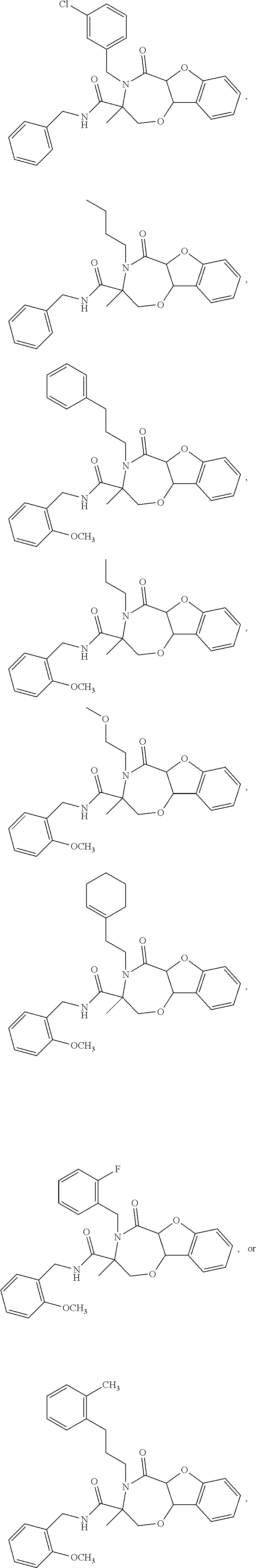

wherein the compound comprises a D in at least one position, and wherein the level of deuterium the at least one position is at least about 50%.

In one embodiment, described herein is a compound of Formula Ia:

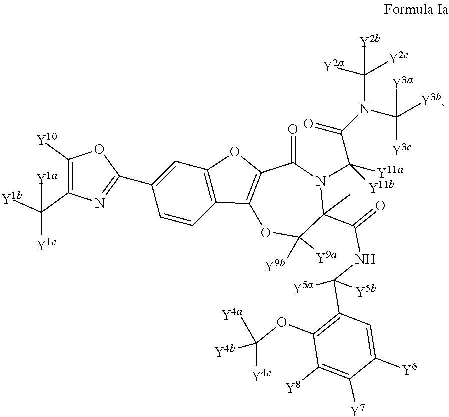

or a pharmaceutically acceptable salt thereof,
wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H or D.

In some embodiments, the compound is of Formula Ia, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, and Y¹⁰ is each independently H. In some embodiments, the compound is of Formula Ia, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, and Y¹⁰ is each independently H. In some embodiments, the compound is of Formula Ia, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y^{4c}, Y^{5a}, Y^{5b}, Y⁶, Y⁷, Y⁸, Y^{9a}, Y^{9b}, and Y¹⁰ is each independently H. In some embodiments, the compound is of Formula Ia, or a pharmaceutically acceptable salt thereof, wherein Y^{4a}, Y^{4b}, and Y^{4c} is each independently D, and Y^{1a}, Y^{1b}, Y^{1c}, Y^{2a}, Y^{2b}, Y^{2c}, Y^{3a}, Y^{3b}, Y^{3c}, Y^{5a}, Y^{5b}, Y⁶, Y⁷, Y⁸, Y^{9a}, Y^{9b}, and Y¹⁰ is each independently H. In some embodiments, the compound is of Formula Ia, or a pharmaceutically acceptable salt thereof, wherein Y^{5a} and Y^{5b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y⁶, Y⁷, Y⁸, Y^{9a}, Y^{9b}, and Y¹⁰ is each independently H. In some embodiments, the compound is of Formula Ia, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁷, Y⁸, Y^{9a}, Y^{9b}, and Y¹⁰ is each independently H. In some embodiments, the compound is of Formula Ia, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁸, Y^{9a}, Y^{9b}, and Y¹⁰ is each independently H. In some embodiments, the compound is of Formula Ia, or a pharmaceutically acceptable salt thereof, wherein Y⁸ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y^{9a}, Y^{9b}, and Y¹⁰ is each independently H. In some embodiments, the compound is of Formula Ia, or a pharmaceutically acceptable salt thereof, wherein Y^{9a} and Y^{9b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, and Y¹⁰ is each independently H. In some embodiments, the compound is of Formula Ia, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, and Y^{9b} is each independently H.

In one embodiment, disclosed herein is a compound of Formula Ib:

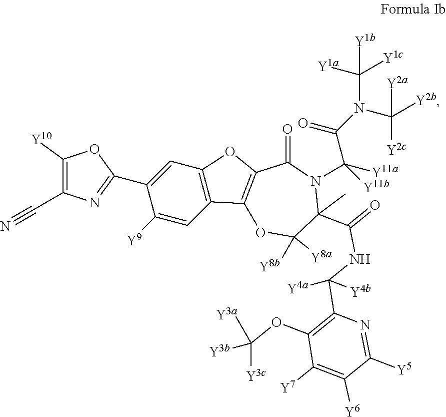

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

In some embodiments, the compound is of Formula Ib, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ib, or a pharmaceutically acceptable salt thereof, wherein Y^{2a}, Y^{2b} and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ib, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ib, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, Y¹⁰ is each independently H. In some embodiments, the compound is of Formula Ib, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, Y¹⁰ is each independently H. In some embodiments, the compound is of Formula Ib, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁷, Y^{8a}, Y^{8b}, Y⁹, Y¹⁰ is each independently H. In some embodiments, the compound is of Formula Ib, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y^{8a}, Y^{8b}, Y⁹, Y¹⁰ is each independently H. In some embodiments, the compound is of Formula Ib, or a pharmaceutically acceptable salt thereof, wherein Y^{8a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y⁹, Y¹⁰ is each independently H. In some embodiments, the compound is of Formula Ib, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y¹⁰ is each independently H. In some embodiments, the compound is of Formula Ib, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, and Y⁹ is each independently H.

In one embodiment, disclosed herein is a compound of Formula Ic:

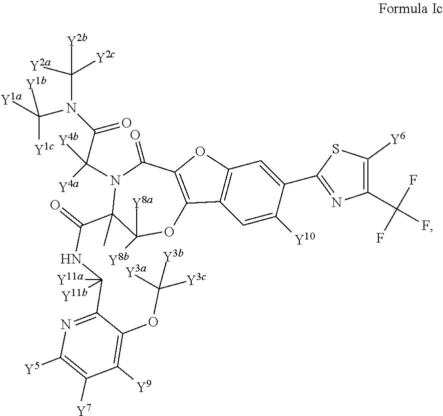

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

In some embodiments, the compound is of Formula Ic, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ic, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ic, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, Y¹⁰ is each independently H. In some embodiments, the compound is of Formula Ic, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, Y¹⁰ is each independently H. In some embodiments, the compound is of Formula Ic, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, Y¹⁰ is each independently H. In some embodiments, the compound is of Formula Ic, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁷, Y^{8a}Y^{8b}, Y⁹, Y¹⁰ is each independently H. In some embodiments, the compound is of Formula Ic, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y^{8a}, Y^{8b}, Y⁹, Y¹⁰ is each independently H. In some embodiments, the compound is of Formula Ic, or a pharmaceutically acceptable salt thereof, wherein Y^{5a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y⁹, Y¹⁰ is each independently H. In some embodiments, the compound is of Formula Ic, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y¹⁰ is each independently H. In some embodiments, the compound is of Formula Ic, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b} and Y⁹ is each independently H.

In one embodiment, disclosed herein is a compound of Formula Id:

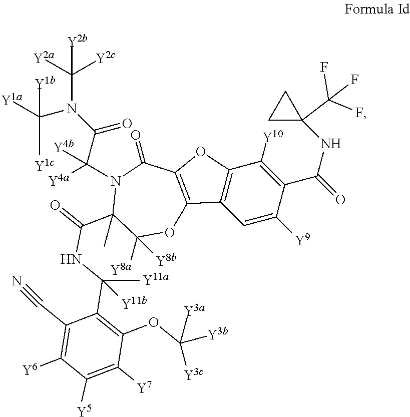

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y^{e}, Y⁶, Y^{8a}, Y^{8b}, Y⁹, Y¹⁰, Y^{11a}, and Y^{11b} is each independently H or D.

In some embodiments, the compound is of Formula Id, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H. In some embodiments, the compound is of Formula Id, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H. In some embodiments, the compound is of Formula Id, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, and Y¹⁰ is each independently H. In some embodiments, the compound is of Formula Id, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, and Y¹⁰ is each independently H. In some embodiments, the compound is of Formula Id, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹ and Y¹⁰ is each independently H. In some embodiments, the compound is of Formula Id, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁷, Y^{8a}, Y^{8b}, Y⁹, and Y¹⁰ is each independently H. In some embodiments, the compound is of Formula Id, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H. In some embodiments, the compound is of Formula Id, or a pharmaceutically acceptable salt thereof, wherein Y^{5a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y⁹, and Y¹⁰ is each independently H. In some embodiments, the compound is of Formula Id, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, and Y¹⁰ is each independently H. In some embodiments, the compound is of Formula Id, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, and Y⁹ is each independently H.

In one embodiment, disclosed herein is a compound of Formula Ie:

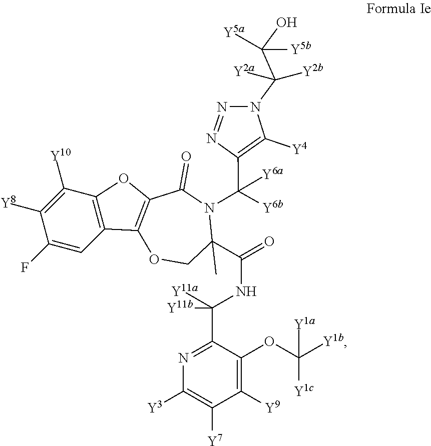

or a pharmaceutically acceptable salt thereof, wherein each Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H or D.

In some embodiments, the compound is of Formula Ie, or a pharmaceutically acceptable salt thereof, wherein Y^{1a}, Y^{1b}, and Y^{1c} is each independently D, and Y^{2a}, Y^{2b}, Y³, Y⁴, Y^{5a}, Y^{5b}, Y^{6a}, Y^{6b}, Y⁷, Y⁸, Y⁹, and Y¹⁰ is each independently H. In some embodiments, the compound is of Formula Ie, or a pharmaceutically acceptable salt thereof, wherein Y^{2a} and Y^{2b} is each independently D, and Y^{1a}, Y^{1b}, Y^{1c}, Y³, Y⁴, Y^{5a}, Y^{5b}, Y^{6a}, Y^{6b}, Y⁷, Y⁸, Y⁹, and Y¹⁰ is each independently H. In some embodiments, the compound is of Formula Ie, or a pharmaceutically acceptable salt thereof, wherein Y³ is D, and Y^{1a}, Y^{1b}, Y^{1c}, Y^{2a}, Y^{2b}, Y⁴, Y^{5a}, Y^{5b}, Y^{6a}, Y^{6b}, Y⁷, Y⁸, Y⁹, and Y¹⁰ is each independently H. In some embodiments, the compound is of Formula Ie, or a pharmaceutically acceptable salt thereof, wherein Y⁴ is D, and Y^{1a}, Y^{1b}, Y^{1c}, Y^{2a}, Y^{2b}, Y³, Y^{5a}, Y^{5b}, Y^{6a}, Y^{6b}, Y⁷, Y⁸, Y⁹, and Y¹⁰ is each independently H. In some embodiments, the compound is of Formula Ie, or a pharmaceutically acceptable salt thereof, wherein Y^{5a} and Y^{5b} is each independently D, and Y^{1a}, Y^{1b}, Y^{1c}, Y^{2a}, Y^{2b}, Y³, Y⁴, Y^{6a}, Y^{6b}, Y⁷, Y⁸, Y⁹, and Y¹⁰ is each independently H. In some embodiments, the compound is of Formula Ie, or a pharmaceutically acceptable salt thereof, wherein Y^{6a} and Y^{6b} is each independently D, and Y^{1a}, Y^{1b}, Y^{1c}, Y^{2a}, Y^{2b}, Y³, Y⁴, Y^{5a}, Y^{5b}, Y⁷, Y⁸, Y⁹, and Y¹⁰ is each independently H. In some embodiments, the compound is of Formula Ie, or a pharmaceutically acceptable salt thereof, wherein Y⁷ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁸, Y⁹, and Y¹⁰ is each independently H. In some embodiments, the compound is of Formula Ie, or a pharmaceutically acceptable salt thereof, wherein Y⁸ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁹, and Y¹⁰ is each independently H. In some embodiments, the compound is of Formula Ie, or a pharmaceutically acceptable salt thereof, wherein Y⁹ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, and Y¹⁰ is each independently H. In some embodiments, the compound is of Formula Ie, or a pharmaceutically acceptable salt thereof, wherein Y¹⁰ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, and is each independently H.

In one embodiment, a compound of Formula I, Ia, Ib, Ic, Id, or Ie, or a pharmaceutically acceptable salt thereof comprises a level of deuterium enrichment denoted D in the at least one position is at least about 70%. In one embodiment, a compound of Formula I, Ia, Ib, Ic, Id, or Ie, or a pharmaceutically acceptable salt thereof comprises a level of deuterium enrichment denoted D in the at least one position is at least about 80%. In one embodiment, a compound of Formula I, Ia, Ib, Ic, Id, or Ie, or a pharmaceutically acceptable salt thereof comprises a level of deuterium enrichment denoted D in the at least one position is at least about 90%. In one embodiment, a compound of Formula I, Ia, Ib, Ic, Id, or Ie, or a pharmaceutically acceptable salt thereof comprises a level of deuterium enrichment denoted D in the at least one position is at least about 98%. In one embodiment, a compound of Formula I, Ia, Ib, Ic, Id, or Ie, or a pharmaceutically acceptable salt thereof comprises a level of deuterium enrichment denoted D in the at least one position is at least about 99.5%.

### Pharmaceutically Acceptable Salts

Salts encompassed within the term “pharmaceutically acceptable salts” refer to the compounds of this disclosure which are generally prepared by reacting the free base or free acid with a suitable organic or inorganic acid, or a suitable organic or inorganic base, respectively, to provide a salt of the compound of the disclosure that is suitable for administration to a subject or patient.

In addition, the compounds of Formula I may also include other salts of such compounds which are not necessarily pharmaceutically acceptable salts, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

Suitable acid addition salts are formed from acids which form non-toxic salts. Examples include, but are not limited to, acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, 1,5-naphathalenedisulfonic acid and xinofoate salts.

Suitable base salts are formed from bases which form non-toxic salts. Examples include, but are not limited to aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see Paulekun, G. S. et al., Trends in Active Pharmaceutical Ingredient Salt Selection Based on Analysis of the Orange Book Database, J. Med. Chem. 2007; 50(26), 6665-6672.

Pharmaceutically acceptable salts of compounds of the disclosure may be prepared by methods well known to one skilled in the art, including but not limited to the following procedures
(i) by reacting a compound of the disclosure with the desired acid or base;
(ii) by removing an acid- or base-labile protecting group from a suitable precursor of a compound of the disclosure or by ring-opening a suitable cyclic precursor, for example, a lactone or lactam, using the desired acid or base; or
(iii) by converting one salt of a compound of the disclosure to another. This may be accomplished by reaction with an appropriate acid or base or by means of a suitable ion exchange procedure.

These procedures are typically carried out in solution. The resulting salt may precipitate out and be collected by filtration or may be recovered by evaporation of the solvent.

### Solvates

The compounds of the disclosure, and pharmaceutically acceptable salts thereof, may exist in unsolvated and solvated forms. The term ‘solvate’ is used herein to describe a molecular complex comprising the compound of the disclosure, or a pharmaceutically acceptable salt thereof, and one or more pharmaceutically acceptable solvent molecules, for example, ethanol. The term ‘hydrate’ is employed when said solvent is water.

In addition, the compounds of Formula I may also include other solvates of such compounds which are not necessarily pharmaceutically acceptable solvates, which may be useful as intermediates for one or more of the following: 1) preparing compounds of Formula I; 2) purifying compounds of Formula I; 3) separating enantiomers of compounds of Formula I; or 4) separating diastereomers of compounds of Formula I.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

### Complexes

Also included within the scope of the disclosure are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, for example, hydrogen bonded complex (cocrystal) may be formed with either a neutral molecule or with a salt. Co-crystals may be prepared by melt crystallization, by recrystallization from solvents, or by physically grinding the components together—see Chem Commun, 17; 1889-1896, by O. Almarsson and M. J. Zaworotko (2004). For a general review of multi-component complexes, see J Pharm Sci, 64(8), 1269-1288, by Haleblian (August 1975).

### Solid Form

The compounds of the disclosure may exist in a continuum of solid states ranging from amorphous to crystalline. The term ‘amorphous’ refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compounds of the disclosure may also exist in a mesomorphic state (mesophase or liquid crystal) when subjected to suitable conditions. The mesomorphic state is intermediate between the true crystalline state and the true liquid state (either melt or solution) and consists of two dimensional order on the molecular level. Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

### Stereoisomers

Compounds of the disclosure may exist as two or more stereoisomers. Stereoisomers of the compounds may include cis and trans isomers (geometric isomers), optical isomers such as R and S enantiomers, diastereomers, rotational isomers, atropisomers, and conformational isomers. For example, compounds of the disclosure containing one or more asymmetric carbon atoms may exist as two or more stereoisomers. Where a compound of the disclosure contains an alkenyl or alkenylene group, geometric cis/trans (or Z/E) isomers are possible. Cis/trans isomers may also exist for saturated rings.

The pharmaceutically acceptable salts of compounds of the disclosure may also contain a counterion which is optically active (e.g., d-lactate or l-lysine) or racemic (e.g., dl-tartrate or dl-arginine).

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where a compound of the disclosure contains an acidic or basic moiety, a base or acid such as 1-phenylethylamine or tartaric acid. The resulting diastereomeric mixture may be separated by chromatography, fractional crystallization, or by using both of said techniques, and one or both of the diastereoisomers converted to the corresponding pure enantiomer(s) by means well known to a skilled person. Chiral compounds of the disclosure (and chiral precursors thereof) may be obtained in enantiomerically-enriched form using chromatography, typically HPLC Concentration of the eluate affords the enriched mixture. Chiral chromatography using sub- and supercritical fluids may be employed. Methods for chiral chromatography useful in some embodiments of the present disclosure are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein).

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two crystal forms are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

### Tautomerism

Where structural isomers are interconvertible via a low energy barrier, tautomeric isomerism (‘tautomerism’) may occur. This may take the form of proton tautomerism in compounds of the disclosure containing, for example, an imino/amino, keto/enol, or oxime/nitroso group, lactam/lactim or so-called valence tautomerism in compounds which contain an aromatic moiety. It follows that a single compound may exhibit more than one type of isomerism.

It must be emphasized that while, for conciseness, the compounds of the disclosure have been drawn herein in a single tautomeric form, all possible tautomeric forms are included within the scope of the disclosure.

### Isotopes

The present disclosure includes all pharmaceutically acceptable isotopically-labeled compounds of the disclosure wherein one or more atoms are replaced by atoms having the same atomic number, but an atomic mass or mass number different from the atomic mass or mass number which predominates in nature.

Examples of isotopes suitable for inclusion in the compounds of the disclosure may include isotopes of hydrogen, such as ²H (D, deuterium) and ³H (T, tritium), carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of the disclosure, for example those incorporating a radioactive isotope, are useful in one or both of drug or substrate tissue distribution studies. The radioactive isotopes, such as, tritium and ¹⁴C are particularly useful for this purpose in view of their ease of incorporation and ready means of detection. Substitution with positron emitting isotopes, such as, ¹¹C, ¹⁸F, ¹⁵O and ¹³N, may be useful in Positron Emission Topography (PET) studies for examining substrate receptor occupancy. Substitution with deuterium may afford certain therapeutic advantages resulting from greater metabolic stability, for example increased in vivo half-life, reduced dosage requirements, reduced CYP450 inhibition (competitive or time dependent), or an improvement in therapeutic index or tolerability.

In some embodiments, the disclosure provides deuterium-labeled (or deuterated) compounds and salts, where the formula and variables of such compounds and salts are each and independently as described herein. “Deuterated” means that at least one of the atoms in the compound is deuterium in an abundance that is greater than the natural abundance of deuterium (typically approximately 0.015%). A skilled artisan recognized that in chemical compounds with a hydrogen atom, the hydrogen atom actually represents a mixture of H and D, with about 0.015% being D. The concentration of the deuterium incorporated into the deuterium-labeled compounds and salt of the disclosure may be defined by the deuterium enrichment factor. It is understood that one or more deuterium may exchange with hydrogen under physiological conditions.

In some embodiments, the deuterium compound is selected from any one of the compounds set forth in TABLE 2-TABLE 6 shown in the Examples section. In some embodiments, one or more hydrogen atoms on certain metabolic sites on the compounds of the disclosure are deuterated.

Isotopically-labeled compounds of the disclosure may generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the disclosure include those wherein the solvent of crystallization may be isotopically substituted, e.g., D₂O, d₆-acetone, d₆-DMSO.

### Prodrugs

A compound of the disclosure may be administered in the form of a prodrug. Thus, certain derivatives of a compound of the disclosure which may have little or no pharmacological activity themselves may, when administered into or onto the body, be converted into a compound of the disclosure having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the disclosure may, for example, be produced by replacing appropriate functionalities present in compounds of the disclosure with certain moieties known to those skilled in the art as ‘pro-moieties’ as described, for example, in ‘Design of Prodrugs’ by H. Bundgaard (Elsevier, 1985).

Thus, a prodrug in accordance with the disclosure may be (a) an ester or amide derivative of a carboxylic acid when present in a compound of the disclosure; (b) an ester, carbonate, carbamate, phosphate or ether derivative of a hydroxyl group when present in a compound of the disclosure; (c) an amide, imine, carbamate or amine derivative of an amino group when present in a compound of the disclosure; (d) a thioester, thiocarbonate, thiocarbamate or sulfide derivatives of a thiol group when present in a compound of the disclosure; or (e) an oxime or imine derivative of a carbonyl group when present in a compound of the disclosure.

Some specific examples of prodrugs in accordance with the disclosure include:
(i) when a compound of the disclosure contains a carboxylic acid functionality (—COOH), an ester thereof, such as a compound wherein the hydrogen of the carboxylic acid functionality of the compound is replaced by C₁-C₈ alkyl (e.g., ethyl) or (C₁-C₈ alkyl)C(═O)OCH₂— (e.g., ^{t}BuC(═O)OCH₂—);
(ii) when a compound of the disclosure contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the disclosure contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the disclosure contains an alcohol functionality (—OH), a phosphate thereof, such as a compound wherein the hydrogen of the alcohol functionality of the compound is replaced by —P(═O)(OH)₂ or —P(═O)(O⁻Na⁺)₂ or —P(═O)(O⁻)₂Ca²⁺;
(v) when a compound of the disclosure contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the disclosure contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.

Certain compounds of the disclosure may themselves act as prodrugs of other compounds the disclosure It is also possible for two compounds of the disclosure to be joined together in the form of a prodrug. In certain circumstances, a prodrug of a compound of the disclosure may be created by internally linking two functional groups in a compound of the disclosure, for instance by forming a lactone.

### Metabolites

Also included within the scope of the disclosure are active metabolites of compounds of the disclosure, that is, compounds formed in vivo upon administration of the drug, often by oxidation or dealkylation. Some examples of metabolites in accordance with the disclosure include, but are not limited to,
(i) where the compound of the disclosure contains an alkyl group, a hydroxyalkyl derivative thereof (—CH>—COH):
(ii) where the compound of the disclosure contains an alkoxy group, a hydroxy derivative thereof (—OR->—OH);
(iii) where the compound of the disclosure contains a tertiary amino group, a secondary amino derivative thereof (—NRR′->—NHR or —NHR′);
(iv) where the compound of the disclosure contains a secondary amino group, a primary derivative thereof (—NHR->—NH₂);
(v) where the compound of the disclosure contains a phenyl moiety, a phenol derivative thereof (-Ph->-PhOH);
(vi) where the compound of the disclosure contains an amide group, a carboxylic acid derivative thereof (—CONH₂->COOH); and
(vii) where the compound contains a hydroxy or carboxylic acid group, the compound may be metabolized by conjugation, for example with glucuronic acid to form a glucuronide. Other routes of conjugative metabolism exist. These pathways are frequently known as Phase 2 metabolism and include, for example, sulfation or acetylation. Other functional groups, such as NH groups, may also be subject to conjugation.

### Pharmaceutical Compositions

In another embodiment, the disclosure comprises pharmaceutical compositions. For pharmaceutical composition purposes, the compound per se or pharmaceutically acceptable salt thereof will simply be referred to as the compounds of the disclosure.

The compositions of this disclosure may be in a variety of forms. These include, for example, liquid, semi-solid and solid dosage forms, such as liquid solutions (e.g., injectable and infusible solutions), dispersions or suspensions, tablets, capsules, pills, powders, liposomes and suppositories. The form depends on the intended mode of administration and therapeutic application.

Typical compositions are in the form of injectable or infusible solutions, such as compositions similar to those used for passive immunization of humans with antibodies in general. One mode of administration is parenteral (e.g., intravenous, subcutaneous, intraperitoneal, intramuscular). In another embodiment, the compound is administered by intravenous infusion or injection. In yet another embodiment, the compound is administered by intramuscular or subcutaneous injection.

Oral administration of a solid dosage form may be, for example, presented in discrete units, such as hard or soft capsules, pills, cachets, lozenges, or tablets, each containing a predetermined amount of at least one compound of the disclosure. In another embodiment, the oral administration may be in a powder or granule form. In another embodiment, the oral dosage form is sub-lingual, such as, for example, a lozenge. In such solid dosage forms, the compounds of the disclosure are ordinarily combined with one or more adjuvants. Such capsules or tablets may comprise a controlled release formulation. In the case of capsules, tablets, and pills, the dosage forms also may comprise buffering agents or may be prepared with enteric coatings.

In another embodiment, oral administration may be in a liquid dosage form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as one or more of wetting, emulsifying, suspending, flavoring (e.g., sweetening), or perfuming agents.

In another embodiment, the disclosure comprises a parenteral dosage form. “Parenteral administration” includes, for example, subcutaneous injections, intravenous injections, intraperitoneally, intramuscular injections, intrasternal injections, and infusion. Injectable preparations (i.e., sterile injectable aqueous or oleaginous suspensions) may be formulated according to the known art using one or more of suitable dispersing, wetting agents, or suspending agents.

In another embodiment, the disclosure comprises a topical dosage form. “Topical administration” includes, for example, dermal and transdermal administration, such as via transdermal patches or iontophoresis devices, intraocular administration, or intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this disclosure are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical excipients include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this disclosure is dissolved or suspended in a suitable excipient. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration, the compounds of the disclosure are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the disclosure comprises a rectal dosage form. Such rectal dosage form may be in the form of, for example, a suppository. Cocoa butter is a traditional suppository base, but various alternatives may be used as appropriate.

Other excipients and modes of administration known in the pharmaceutical art may also be used. Pharmaceutical compositions of the disclosure may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; Rowe, Raymond C. Handbook of Pharmaceutical Excipients. Chicago, Pharmaceutical Press, 2005; Stahl, P. Heinrich and Camilli G. Wermuth, Eds. Handbook of Pharmaceutical Salts: Properties, Selection, and Use. New York: Wiley-VCH, 2011; and Brittain, Harry G., Ed. Polymorphism in Pharmaceutical Solids. New York: Informa Healthcare USA, Inc., 2016.

Acceptable excipients are nontoxic to subjects at the dosages and concentrations employed, and may comprise one or more of the following: 1) buffers such as phosphate, citrate, or other organic acids; 2) salts such as sodium chloride; 3) antioxidants such as ascorbic acid or methionine; 4) preservatives such as octadecyldimethylbenzyl ammonium chloride, hexamethonium chloride, benzalkonium chloride, benzethonium chloride, phenol, butyl or benzyl alcohol; 5) alkyl parabens such as methyl or propyl paraben, catechol, resorcinol, cyclohexanol, 3-pentanol, or m-cresol; 6) low molecular weight (less than about 10 residues) polypeptides; 7) proteins such as serum albumin, gelatin, or immunoglobulins; 8) hydrophilic polymers such as polyvinylpyrrolidone; 9) amino acids such as glycine, glutamine, asparagine, histidine, arginine, or lysine; 10) monosaccharides, disaccharides, or other carbohydrates including glucose, mannose, or dextrins; 11) chelating agents such as EDTA; 12) sugars such as sucrose, mannitol, trehalose or sorbitol; 13) salt-forming counter-ions such as sodium, metal complexes (e.g., Zn-protein complexes), or 14) non-ionic surfactants such as polysorbates (e.g., polysorbate 20 or polysorbate 80), poloxamers or polyethylene glycol (PEG).

For oral administration, the compositions may be provided in the form of tablets or capsules containing 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Dosing regimens may depend on the route of administration, dose scheduling, and use of flat-dose, body surface area or weight-based dosing. For example, for weight-based dosing, intravenously doses may range from about 0.01 to about 10 mg/kg/minute during a constant rate infusion.

Liposome containing compounds of the disclosure may be prepared by methods known in the art (See, for example, Chang, H. I.; Yeh, M. K.; Clinical development of liposome-based drugs: formulation, characterization, and therapeutic efficacy; Int J Nanomedicine 2012; 7; 49-60). Particularly useful liposomes may be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

Compounds of the disclosure may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 20th Ed., Mack Publishing (2000).

Sustained-release preparations may be used. Suitable examples of sustained-release preparations include semi-permeable matrices of solid hydrophobic polymers containing a compound of the disclosure, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides, copolymers of L-glutamic acid and 7 ethyl-L-glutamate, non-degradable ethylene-vinyl acetate, degradable lactic acid-glycolic acid copolymers such as those used in leuprolide acetate for depot suspension (injectable microspheres composed of lactic acid-glycolic acid copolymer and leuprolide acetate), sucrose acetate isobutyrate, and poly-D-(−)-3-hydroxybutyric acid.

The formulations to be used for intravenous administration must be sterile. This is readily accomplished by, for example, filtration through sterile filtration membranes. Compounds of the disclosure are generally placed into a container having a sterile access port, for example, an intravenous solution bag or vial having a stopper pierceable by a hypodermic injection needle.

Suitable emulsions may be prepared using commercially available fat emulsions, such as a lipid emulsions comprising soybean oil, a fat emulsion for intravenous administration (e.g., comprising safflower oil, soybean oil, egg phosphatides and glycerin in water), emulsions containing soya bean oil and medium-chain triglycerides, and lipid emulsions of cottonseed oil. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion may comprise fat droplets between 0.1 and 1.0 μm, particularly 0.1 and 0.5 μm, and have a pH in the range of 5.5 to 8.0.

For example, the emulsion compositions may be those prepared by mixing a compound of the disclosure with a lipid emulsions comprising soybean oil or the components thereof (soybean oil, egg phospholipids, glycerol and water).

Compositions for inhalation or insufflation include solutions and suspensions in pharmaceutically acceptable aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

A drug product intermediate (DPI) is a partly processed material that must undergo further processing steps before it becomes bulk drug product. Compounds of the disclosure may be formulated into drug product intermediate DPI containing the active ingredient in a higher free energy form than the crystalline form. One reason to use a DPI is to improve oral absorption characteristics due to low solubility, slow dissolution, improved mass transport through the mucus layer adjacent to the epithelial cells, and in some cases, limitations due to biological barriers such as metabolism and transporters. Other reasons may include improved solid state stability and downstream manufacturability. In one embodiment, the drug product intermediate contains a compound of the disclosure isolated and stabilized in the amorphous state (for example, amorphous solid dispersions (ASDs)). There are many techniques known in the art to manufacture ASD's that produce material suitable for integration into a bulk drug product, for example, spray dried dispersions (SDD's), melt extrudates (often referred to as HME's), co-precipitates, amorphous drug nanoparticles, and nano-adsorbates. In one embodiment amorphous solid dispersions comprise a compound of the disclosure and a polymer excipient. Other excipients as well as concentrations of said excipients and the compound of the disclosure are well known in the art and are described in standard textbooks. See, for example, “Amorphous Solid Dispersions Theory and Practice” by Navnit Shah et al.

### Administration and Dosing

Typically, a compound of the disclosure is administered in an amount effective to treat a condition as described herein. The compounds of the disclosure may be administered as compound per se, or alternatively, as a pharmaceutically acceptable salt. For administration and dosing purposes, the compound per se or pharmaceutically acceptable salt thereof will simply be referred to as the compounds of the disclosure.

The compounds of the disclosure are administered by any suitable route in the form of a pharmaceutical composition adapted to such a route, and in a dose effective for the treatment intended. The compounds of the disclosure may be administered orally, rectally, vaginally, parenterally, topically, intranasally, or by inhalation.

The compounds of the disclosure may be administered orally. Oral administration may involve swallowing, so that the compound enters the gastrointestinal tract, or buccal or sublingual administration may be employed by which the compound enters the bloodstream directly from the mouth. In some embodiments, a compound for Formula I, Ia, Ib, Ic, Id, or Ie, or a pharmaceutically acceptable salt thereof, is administered orally.

In another embodiment, the compounds of the disclosure may also be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques. In some embodiments, a compound for Formula I, Ia, Ib, Ic, Id, or Ie, or a pharmaceutically acceptable salt thereof, is administered parenterally. In some embodiments, a compound for Formula I, Ia, Ib, Ic, Id, or Ie, or a pharmaceutically acceptable salt thereof, is administered intravenously. In some embodiments, a compound for Formula I, Ia, Ib, Ic, Id, or Ie, or a pharmaceutically acceptable salt thereof, is administered subcutaneously. In some embodiments, a compound for Formula I, la, Ib, Ic, Id, or Ie, or a pharmaceutically acceptable salt thereof, is administered intramuscularly.

In another embodiment, the compounds of the disclosure may also be administered topically to the skin or mucosa, that is, dermally or transdermally. In another embodiment, the compounds of the disclosure may also be administered intranasally or by inhalation. In another embodiment, the compounds of the disclosure may be administered rectally or vaginally. In another embodiment, the compounds of the disclosure may also be administered directly to the eye or ear.

The dosage regimen for the compounds of the disclosure or compositions containing said compounds is based on a variety of factors, including the type, age, weight, sex and medical condition of the patient; the severity of the condition; the route of administration; and the activity of the particular compound employed. Thus, the dosage regimen may vary widely. In one embodiment, the total daily dose of a compound of the disclosure is typically from about 0.01 to about 100 mg/kg (i.e., mg compound of the disclosure per kg body weight) for the treatment of the indicated conditions discussed herein. In another embodiment, total daily dose of the compound of the disclosure is from about 0.1 to about 50 mg/kg, and in another embodiment, from about 0.5 to about 30 mg/kg.

In some embodiments, a compound of Formula I, Ia, Ib, Ic, Id, or Ie or a pharmaceutically acceptable salt thereof, or a pharmaceutical composition comprising a compound of Formula I, Ia, Ib, Ic, Id, or Ie or a pharmaceutically acceptable salt thereof may provide the compound in an amount of from about 0.01 mg to about 150 mg, from about 150 mg to about 250 mg, from about 250 mg to about 500 mg, from about 500 mg to about 750 mg, from about 750 mg to about 1000 mg, from about 1250 mg to about 1500 mg, from about 1500 mg to about 1750 mg, from about 1750 mg to about 2000 mg, from about 2000 mg to about 2250 mg, from about 2250 mg to about 2500 mg, from about 2500 mg to about 2750 mg, from about 2750 mg to about 3000 mg, from about 3000 mg to about 3250 mg, from about 3250 mg to about 3500 mg, from about 3500 mg to about 3750 mg, from about 3750 mg to about 4000 mg, from about 4000 mg to about 4250 mg, from about 4250 mg to about 4500 mg, from about 4500 mg to about 4750 mg, or from about 4750 mg to about 5000 mg. In one embodiment, the compound or pharmaceutically acceptable salt thereof is provided in an amount of from about 1 mg to about 2500 mg. In one embodiment, the compound or pharmaceutically acceptable salt thereof is provided in an amount of from about 1 mg to about 100 mg. In one embodiment, the compound or pharmaceutically acceptable salt thereof is provided in an amount of from about 150 mg to about 2500 mg. In one embodiment, the compound or pharmaceutically acceptable salt thereof is provided in an amount of from about 150 mg to about 500 mg. In one embodiment, the compound or pharmaceutically acceptable salt thereof is provided in an amount of from about 100 mg to about 1000 mg. In one embodiment, the compound or pharmaceutically acceptable salt thereof is provided in an amount of from about 500 mg to about 1500 mg. In one embodiment, the compound or pharmaceutically acceptable salt thereof is provided in an amount of from about 1500 mg to about 2500 mg. In one embodiment, the compound or pharmaceutically acceptable salt thereof is provided in an amount of from about 2500 mg to about 5000 mg.

A compound of Formula I, Ia, Ib, Ic, Id, or Ie or a pharmaceutically acceptable salt thereof, or a pharmaceutical composition comprising a compound of Formula I, Ia, Ib, Ic, Id, or Ie or a pharmaceutically acceptable salt thereof may provide the compound in an amount of about 0.01 mg, about 50 mg, about 100 mg, about 150 mg, about 200 mg, about 250 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg. In some embodiments, a compound of Formula I, Ia, Ib, Ic, Id, or Ie or a pharmaceutically acceptable salt thereof may be provided in an amount of about 25 mg. In some embodiments, a compound of Formula I, Ia, Ib, Ic, Id, or Ie or a pharmaceutically acceptable salt thereof may be provided in an amount of about 50 mg. In some embodiments, a compound of Formula I, Ia, Ib, Ic, Id, or Ie or a pharmaceutically acceptable salt thereof may be provided in an amount of about 75 mg. In some embodiments, a compound of Formula I, Ia, Ib, Ic, Id, or Ie or a pharmaceutically acceptable salt thereof may be provided in an amount of about 100 mg. In some embodiments, a compound of Formula I, Ia, Ib, Ic, Id, or Ie or a pharmaceutically acceptable salt thereof may be provided in an amount of about 250 mg. In some embodiments, a compound of Formula I, Ia, Ib, Ic, Id, or Ie or a pharmaceutically acceptable salt thereof may be provided in an amount of about 500 mg. In some embodiments, a compound of Formula I, Ia, Ib, Ic, Id, or Ie or a pharmaceutically acceptable salt thereof may be provided in an amount of about 1000 mg.

It is not uncommon that the administration of the compounds of the disclosure will be repeated a plurality of times in a day (typically no greater than 4 times). Multiple doses per day typically may be used to increase the total daily dose, if desired. In one embodiment, a compound of Formula I, Ia, Ib, Ic, Id, or Ie, or a pharmaceutically acceptable salt thereof, is administered once a day. In one embodiment, a compound of Formula I, Ia, Ib, Ic, Id, or Ie, or a pharmaceutically acceptable salt thereof, is administered twice a day. In one embodiment, a compound of Formula I, Ia, Ib, Ic, Id, or Ie, or a pharmaceutically acceptable salt thereof, is administered three times a day.

### Therapeutic Methods and Uses

The compounds of the disclosure may induce KLF2 and may be used to treat a condition. In some embodiments, a compound of the disclosure may increase KLF2 and regulate protective functions of the endothelium, for example, vasodilatory, anti-thrombotic, antioxidant, or anti-inflammatory activities. In some embodiments, a compound of the disclosure may increase KLF2 and prevent atherosclerosis. In some embodiments, a compound of the disclosure may increase KLF2 and reverse atherosclerosis.

In one embodiment, a compound of the Formula I, Ia, Ib, Ic, Id, or Ie, or a pharmaceutically acceptable salt thereof, can be used to treat an inflammatory disease. In one embodiment, a compound of Formula I, Ia, Ib, Ic, Id, or Ie, or a pharmaceutically acceptable salt thereof, can be used to treat endothelial dysfunction. In some embodiments, the inflammatory disease or endothelial dysfunction is selected from the group consisting of atherosclerosis, coronary artery disease, stroke, peripheral arterial disease, coronary microvascular diseases, angina, systemic hypertension, pulmonary arterial hypertension, heart failure, and diabetic microvascular diseases, such as diabetic nephropathy, diabetic retinopathy or diabetic neuropathy, an autoimmune disease, inflammatory disease, infectious diseases, arterial and venous thrombosis, long covid, ischemia with non-obstructive coronary arteries (INOCA), antiphospholipid syndrome (APS), and chronic kidney disease. In some embodiments, a compound of the Formula I, Ia, Ib, Ic, Id, or Ie, or a pharmaceutically acceptable salt thereof, can be used to treat atherosclerosis. In some embodiments, a compound of the Formula I, Ia, Ib, Ic, Id, or Ie, or a pharmaceutically acceptable salt thereof, can be used to treat coronary artery disease. In some embodiments, a compound of the Formula I, Ia, Ib, Ic, Id, or Ie, or a pharmaceutically acceptable salt thereof, can be used to treat peripheral arterial disease.

### Co-Administration

The compounds of the disclosure may be used alone, or in combination with one or more other therapeutic agents. The disclosure provides any of the uses, methods or compositions as defined herein wherein the compound of the disclosure, or pharmaceutically acceptable salt thereof, is used in combination with one or more other therapeutic agent discussed herein.

The administration of two or more compounds “in combination” means that all of the compounds are administered closely enough in time to affect treatment of the subject. The two or more compounds may be administered simultaneously or sequentially, via the same or different routes of administration, on same or different administration schedules and with or without specific time limits depending on the treatment regimen. Additionally, simultaneous administration may be carried out by mixing the compounds prior to administration or by administering the compounds at the same point in time but as separate dosage forms at the same or different site of administration. Examples of “in combination” include, but are not limited to, “concurrent administration,” “co-administration,” “simultaneous administration,” “sequential administration” and “administered simultaneously”.

A compound of the disclosure and the one or more other therapeutic agents may be administered as a fixed or non-fixed combination of the active ingredients. The term “fixed combination” means a compound of the disclosure, or a pharmaceutically acceptable salt thereof, and the one or more therapeutic agents, are both administered to a subject simultaneously in a single composition or dosage. The term “non-fixed combination” means that a compound of the disclosure, or a pharmaceutically acceptable salt thereof, and the one or more therapeutic agents are formulated as separate compositions or dosages such that they may be administered to a subject in need thereof simultaneously or at different times with variable intervening time limits, wherein such administration provides effective levels of the two or more compounds in the body of the subject.

In one embodiment, a compound of the disclosure can be administered in combination with a cardiovascular drug, for example, a cardiovascular drug that improves endothelial function. In some embodiments, the cardiovascular drug is an angiotensin-converting enzyme (ACE) inhibitor, an angiotensin II receptor type 1 (AT1) blocker, an antioxidant agent, a beta blocker, a dihydropyridine calcium channel blocker, a phosphodiesterase-5 (PDE5) inhibitor, a statin, a proprotein convertase subtilisin/kexin type 9 (PCSK9) inhibitor, angiotensin-(1-7), bradykinin, an eNOS transcription enhancer, an I_{f} inhibitor, or sphingosine-1-phosphate (S1P), including the pharmaceutically acceptable salts of the agents and the pharmaceutically acceptable solvates of said agents and salts.

In some embodiments, the ACE inhibitor is benazepril, captopril, enalapril, fosinopril, lisinopril, moexipril, perindopril, quinapril, ramipril, or trandolapril, or a pharmaceutically acceptable salt thereof or a pharmaceutically acceptable solvate of said ACE inhibitor or salt thereof. In some embodiments, the AT1 blocker is azilsartan, candesartan, eprosartan, irbesartan, losartan, olmesartan, telmisartan, or valsartan, or a pharmaceutically acceptable salt thereof or a pharmaceutically acceptable solvate of said AT1 blocker or salt thereof. In some embodiments, the antioxidant agent is vitamin C, vitamin E, N-acetylcysteine, or genistein, or a pharmaceutically acceptable salt thereof or a pharmaceutically acceptable solvate of said antioxidant or salt thereof. In some embodiments, the beta blocker is a β1-selective blocker, for example, nebivolol; or a non-selective β1- and β2 antagonist, for example, carvedilol, or a pharmaceutically acceptable salt thereof or a pharmaceutically acceptable solvate of said beta blocker or salt thereof. In some embodiments, the dihydropyridine calcium channel blocker is nicardipine, nifedipine, israpidine, amlodipine, or azelnidipine, or a pharmaceutically acceptable salt thereof or a pharmaceutically acceptable solvate of said dihydropyridine calcium channel blocker or salt thereof.

In some embodiments, the PDE5 inhibitor is sildenafil, tadalafil, or vardenafil, or a pharmaceutically acceptable salt thereof or a pharmaceutically acceptable solvate of said PDE5 inhibitor or salt thereof. In some embodiments, the statin is pravastatin, atorvastatin, fluvastatin, lovastatin, pitavastatin, rosuvastatin, or simvastatin, or a pharmaceutically acceptable salt thereof or a pharmaceutically acceptable solvate of said statin or salt thereof. In some embodiments, the PSK9 inhibitor is alirocumab, evolocumab, or inclisiran, or a pharmaceutically acceptable salt thereof or a pharmaceutically acceptable solvate of said PSK9 inhibitor or salt thereof.

In one embodiment, the present disclosure provides a pharmaceutical composition comprising a compound of Formula I, Ia, Ib, Ic, Id, or Ie or a pharmaceutically acceptable salt thereof, wherein the pharmaceutical composition is administered in combination with a pharmaceutical composition comprising a cardiovascular drug selected from the group consisting of an angiotensin-converting enzyme (ACE) inhibitor, an angiotensin II receptor type 1 (AT1) blocker, an antioxidant agent, a beta blocker, a dihydropyridine calcium channel blocker, a phosphodiesterase-5 (PDE5) inhibitor, a statin, a proprotein convertase subtilisin/kexin type 9 (PCSK9) inhibitor, angiotensin-(1-7), bradykinin, an endothelial nitric oxide synthase (eNOS) transcription enhancer, an I_{f} inhibitor, or sphingosine-1-phosphate (S1P), including the pharmaceutically acceptable salts of the cardiovascular drug and the pharmaceutically acceptable solvates of said cardiovascular drug and salts. In some embodiments, the compound of Formula I, Ia, Ib, Ic, Id, or Ie or a pharmaceutically acceptable salt thereof and a cardiovascular drug disclosed herein can be administered simultaneously. In some embodiments, the compound of Formula I, Ia, Ib, Ic, Id, or Ie or a pharmaceutically acceptable salt thereof and a cardiovascular drug disclosed herein can be administered at different times.

These agents and compounds of the disclosure may be combined with pharmaceutically acceptable vehicles such as saline, Ringer's solution, dextrose solution, and the like. The particular dosage regimen, i.e., dose, timing and repetition, will depend on the particular individual and that individual's medical history.

### Kits

Another aspect of the invention provides kits comprising a compound of the disclosure or pharmaceutical compositions comprising a compound of the disclosure. A kit may include, in addition to a compound of the disclosure or pharmaceutical composition thereof, diagnostic or therapeutic agents. A kit may also include instructions for use in a diagnostic or therapeutic method. In some embodiments, the kit includes the compound or a pharmaceutical composition thereof and a diagnostic agent. In other embodiments, the kit includes the compound or a pharmaceutical composition thereof and one or more therapeutic agents, such as a cardiovascular drug disclosed herein.

In yet another embodiment, the disclosure comprises kits that are suitable for use in performing the methods of treatment described herein. In one embodiment, the kit contains a first dosage form comprising one or more of the compounds of the disclosure in quantities sufficient to carry out the methods of the disclosure. In another embodiment, the kit comprises one or more compounds of the disclosure in quantities sufficient to carry out the methods of the disclosure and a container for the dosage and a container for the dosage.

### Synthetic Methods

Compounds of the present disclosure may be synthesized by synthetic routes that include processes analogous to those well-known in the chemical arts, particularly in light of the description contained herein. The starting materials are generally available from commercial sources or may be prepared using methods well known to those skilled in the art. Many of the compounds used herein, are related to, or may be derived from compounds in which one or more of the scientific interest or commercial need has occurred. Accordingly, such compounds may be one or more of 1) commercially available; 2) reported in the literature or 3) prepared from other commonly available substances by one skilled in the art using materials which have been reported in the literature.

For illustrative purposes, the reaction schemes depicted below provide potential routes for synthesizing the compounds of the present disclosure as well as key intermediates. For a more detailed description of the individual reaction steps, see the Examples section below. Those skilled in the art will appreciate that other synthetic routes may be used to synthesize the inventive compounds. Although specific starting materials and reagents are discussed below, other starting materials and reagents may be substituted to provide one or more of a variety of derivatives or reaction conditions. In addition, many of the compounds prepared by the methods described below may be further modified in light of this disclosure using conventional chemistry well known to those skilled in the art.

The skilled person will appreciate that the experimental conditions set forth in the schemes that follow are illustrative of suitable conditions for effecting the transformations shown, and that it may be necessary or desirable to vary the precise conditions employed for the preparation of compounds of the disclosure. It will be further appreciated that it may be necessary or desirable to carry out the transformations in a different order from that described in the schemes, or to modify one or more of the transformations, to provide the desired compound of the disclosure.

In the preparation of compounds of the disclosure it is noted that some of the preparation methods useful for the preparation of the compounds described herein may require protection of remote functionality (e.g., a primary amine, secondary amine, carboxyl, etc. in a precursor of a compound of the disclosure). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see March's Advanced Organic Chemistry: Reactions, Mechanisms, and Structure 8th Edition.

For example, if a compound contains a amine or carboxylic acid functionality, such functionality may interfere with reactions at other sites of the molecule if left unprotected. Accordingly, such functionalities may be protected by an appropriate protecting group (PG) which may be removed in a subsequent step. Suitable protecting groups for amine and carboxylic acid protection include those protecting groups commonly used in peptide synthesis (such as N-t-butoxycarbonyl (Boc), benzyloxycarbonyl (Cbz), and 9-fluorenylmethylenoxycarbonyl (Fmoc) for amines and lower alkyl or benzyl esters for carboxylic acids) which are generally not chemically reactive under the reaction conditions described and may typically be removed without chemically altering other functionality in a compound of the disclosure.

### General Experimental Methods

In the non-limiting Examples and Preparations that illustrate the invention and that are set out in the description, the following the abbreviations, definitions and analytical procedures may be referred to. Other abbreviations common in the art may also be used. Compounds of the present invention were named using ChemDraw Professional™ version 20 (Perkin Elmer) or were given names which are consistent with IUPAC nomenclature.

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g., s, singlet; d, doublet; t, triplet; q, quartet; quin, quintet; m, multiplet; br, broad. The following abbreviations have been used for common NMR solvents: CD₃CN, deuteroacetonitrile; CDCl₃, deuterochloroform; DMSO-d₆, deuterodimethylsulfoxide; and MeOD, deuteromethanol. Where appropriate, tautomers may be recorded within the NMR data; and some exchangeable protons may not be visible. Some resonances in the NMR spectrum appear as complex multiplets because the isolate is a mixture of two conformers.

Mass spectra were recorded using electron impact ionization (EI), electrospray ionization (ESI) or atmospheric pressure chemical ionization (APCI). The observed ions are reported as MS m/z and may be positive ions of the compound [M]⁺, compound plus a proton [M+H]⁺, or compound plus a sodium ion [M+Na]⁺. In some cases the only observed ions may be fragment ions reported as [M+H-(fragment lost)]⁺. Where relevant, the reported ions are assigned for isotopes of chlorine (³⁵Cl and/or ³⁷Cl), bromine (⁷⁹Br and/or ⁸¹Br) and tin (¹²⁰Sn).

Wherein TLC, chromatography or HPLC has been used to purify compounds, one skilled in the art may choose any appropriate solvent or combination of solvents to purify the desired compound. Chromatographic separations (excluding HPLC) were carried out using silica gel adsorbent unless otherwise noted.

All reactions were carried out using continuous stirring under an atmosphere of nitrogen or argon gas unless otherwise noted. In some cases, reactions were purged with nitrogen or argon gas prior to the start of the reaction. In these cases, the nitrogen or argon gas was bubbled through the liquid phase of the mixture for the approximate specified time. Solvents used were commercial anhydrous grades. All starting materials were commercially available products. In some cases, starting materials were prepared according to reported literature procedures. It will be apparent to one skilled in the art that the word “concentrated” as used herein generally refers to the practice of evaporation of solvent under reduced pressure, typically accomplished using a rotary evaporator.

The Schemes described below are intended to provide a general description of the methodology employed in the preparation of the compounds of the present disclosure. In the following Schemes, the general methods for the preparation of the compounds are shown either in racemic or enantioenriched form.

It will be apparent to one skilled in the art that all of the synthetic transformations may be conducted in a precisely similar manner whether the materials are enantioenriched or racemic. Moreover, the resolution to the desired optically active material may take place at any desired point in the sequence using well known methods such as described herein and in the chemistry literature.

### EXAMPLES

In order that this disclosure may be better understood, the following examples are set forth. These examples are for purposes of illustration only and are not to be construed as limiting the scope of the disclosure in any manner.

The compounds of the invention comprise a compound of TABLE 1 comprising a D in at least one position, wherein the level of deuterium at the at least one position is at least about 50%. In some embodiments, the level of deuterium enrichment denoted D is at least about 80%. In some embodiments, the level of deuterium enrichment denoted D is at least about 90%. In some embodiments, the level of deuterium enrichment denoted D is at least about 98%. The compounds in TABLE 1 were prepared using the experimental methods described in PCT/US2023/012487, which is incorporated herein by reference.

Synthetic methods for synthesizing examples 36, 101, 102, 108, 109, 113, 125, and 126 are summarized as follows:

### Example 36: N-(2-(methoxy-d₃)benzyl)-4-(2-methoxyethyl)-3-methyl-5-oxo-2,3,4,5-tetrahydrobenzofuro[2,3-f][1,4]oxazepine-3-carboxamide

### Intermediate BB-2

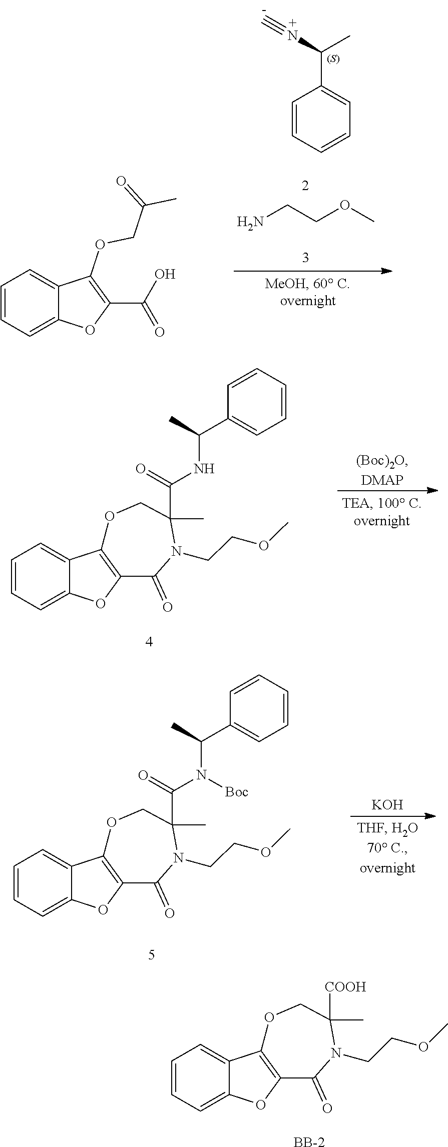

A mixture of 3-(2-oxopropoxy)benzofuran-2-carboxylic acid (220 g, 0.94 mol, 1.0 eq), 2-methoxyethylamine (90 g, 1.13 mol, 1.2 eq), and (S)-(1-isocyanoethyl)benzene (160 g, 1.22 mol, 1.3 eq) in MeOH (1 L) was stirred at 60° C. overnight and concentrated under reduced pressure. The crude product was by column chromatography on silica gel (Hex/EtOAc, 100:1 to 50:1) to give 4-(2-methoxyethyl)-3-methyl-5-oxo-N—((S)-1-phenylethyl)-2,3,4,5-tetrahydrobenzofuro[2,3-f][1,4]oxazepine-3-carboxamide (240 g, 60%) as a light-yellow solid. LC-MS: 423.1 [M+H]⁺; TLC:PE/EA=1:1, UV; R_{f}(compound 1)=0.70; R_{f} (compound 4)=0.40.

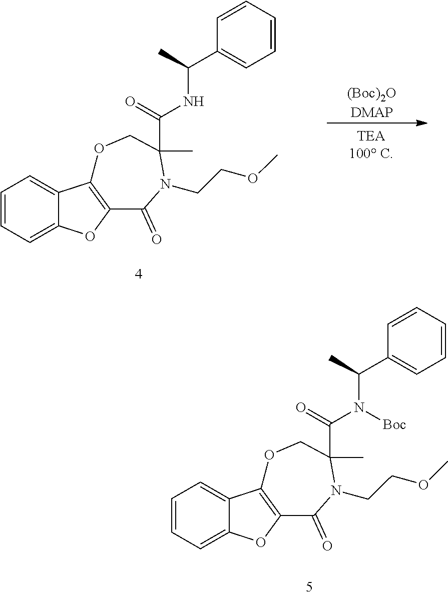 [SMILES: COCCN1C(=O)c2oc3ccccc3c2OCC1(C)C(=O)N[C@@H](C)c1ccccc1 ; COCCN1C(=O)c2oc3ccccc3c2OCC1(C)C(=O)N(C(=O)OC(C)(C)C)[C@@H](C)c1ccccc1]

A mixture of 4-(2-methoxyethyl)-3-methyl-5-oxo-N—((S)-1-phenylethyl)-2,3,4,5-tetrahydrobenzofuro[2,3-f][1,4]oxazepine-3-carboxamide (240 g, 0.57 mol, 1.0 eq), (Boc)₂O (496 g, 2.28 mol, 4.0 eq), and DMAP (69 g, 0.57 mol, 1.0 eq) in TEA (500 mL) was stirred at 110° C. overnight. The mixture was diluted with EtOAc (500 mL). The organic layer was washed with brine, dried over anhydrous Na₂SO₄, filtered, and concentrated under reduced pressure. The residue was purified by column chromatography on silica gel (PE:EtOAc=1:1) to give tert-butyl (4-(2-methoxyethyl)-3-methyl-5-oxo-2,3,4,5-tetrahydrobenzofuro[2,3-f][1,4]oxazepine-3-carbonyl)((S)-1-phenylethyl)carbamate (160 g, 54%) as a light-yellow solid. LC-MS: 523.1 [M+H]⁺; TLC: PE/EA=1:1, UV; R_{f} (compound 4)=0.40; R_{f} (compound 5)=0.70.

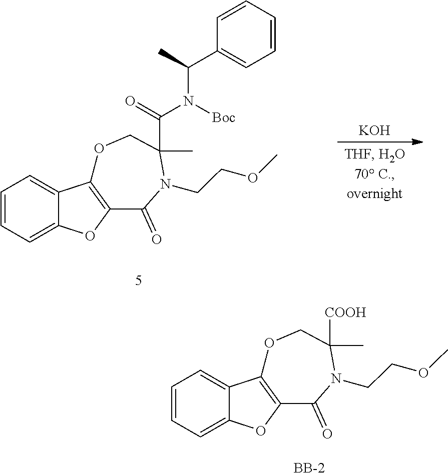

To a solution of tert-butyl (4-(2-methoxyethyl)-3-methyl-5-oxo-2,3,4,5-tetrahydrobenzofuro[2,3-f][1,4]oxazepine-3-carbonyl)((S)-1-phenylethyl)carbamate (160 g, 0.31 mol, 1.0 eq) in EtOH (500 mL), THF (250 mL) was added KOH solution (10% w/w in H₂O, 500 mL). The reaction mixture was stirred at 70° C. overnight, added 6 N HCl to adjust to pH 1-2. The mixture was filtered and the cake was washed with water (200 mL). The solid was dried at 25° C. under reduced pressure to give the desired product (76 g, 23%). LC-MS: 320.1[M+1]+; 1H NMR (400 MHz, DMSO) δ 13.28 (s, 1H), 7.68 (d, J=7.6 Hz, 1H), 7.61 (d, J=8.4 Hz, 1H), 7.51 (ddd, J=8.4, 7.2, 1.3 Hz, 1H), 7.38-7.24 (m, 1H), 4.83 (d, J=12.3 Hz, 1H), 4.38 (d, J=12.3 Hz, 1H), 3.83-3.72 (m, 1H), 3.64 (dt, J=14.5, 6.1 Hz, 1H), 3.54-3.41 (m, 2H), 3.23 (s, 3H), 1.74 (s, 3H).

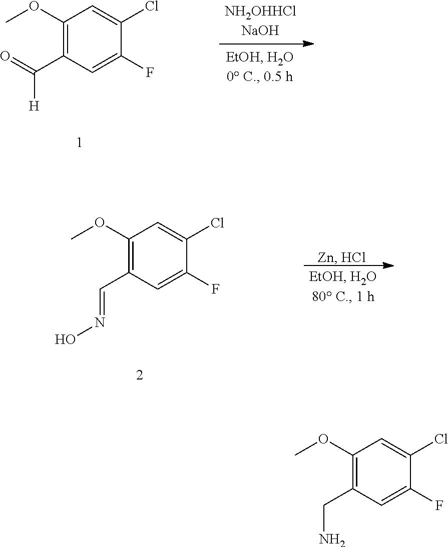

(4-chloro-5-fluoro-2-methoxyphenyl)methanamine: A mixture of NaOH (192 mg, 4.8 mmol, 3.0 eq.) and hydroxylamine hydrochloride (167 mg, 2.4 mmol, 1.5 eq.) was dissolved in 1 mL of water and 5 mL of ethanol at 0° C. for 5 min. To the above mixture, 4-chloro-5-fluoro-2-methoxybenzaldehyde (300 mg, 1.6 mmol, 1.0 eq.) was added. The reaction mixture was warmed to room temperature and stirred for 1 h. At the end of the reaction (confirmed by TLC) the reaction was diluted with water (10 mL) and the aqueous phase was extracted with EA (3×10 mL). The organic phases were washed with brine (10 mL), dried over anhydrous Na2SO4 and concentrated under reduced pressure to yield the crude product (E)-4-chloro-5-fluoro-2-methoxybenzaldehyde oxime (300 mg, 92%) as a white solid. LC-MS: 204.1[M+H]⁺ (V1638-085); TLC: PE/EA=10:1, UV; R_{f}(compound 1)=0.70; R_{f} (compound 2)=0.50.

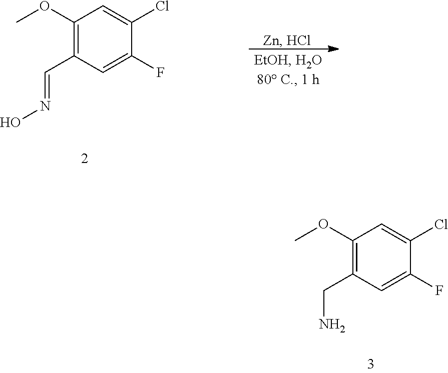

(E)-4-chloro-5-fluoro-2-methoxybenzaldehyde oxime (300 mg, 1.5 mmol) was dissolved in EtOH (6 mL) and water (1 mL). concentrated HCl (2 mL) was added dropwise at 0° C. and the mixture was stirred 10 min before Zn powder (780 mg, 12 mmol, 8 eq.) was added. The mixture was stirred 1 h at 80° C. The mixture was cooled to room temperature and filtered. The filtrate was concentrated under reduced pressure to give a white solid (4-chloro-5-fluoro-2-methoxyphenyl)methanamine (280 mg, 98%). The crude product was used directly in the next step without further purification. TLC: PE/EA=10:1, UV; R_{f}(compound 2)=0.50; R_{f} (compound 3)=0.00; LC-MS: 190.1 [M+H]⁺.

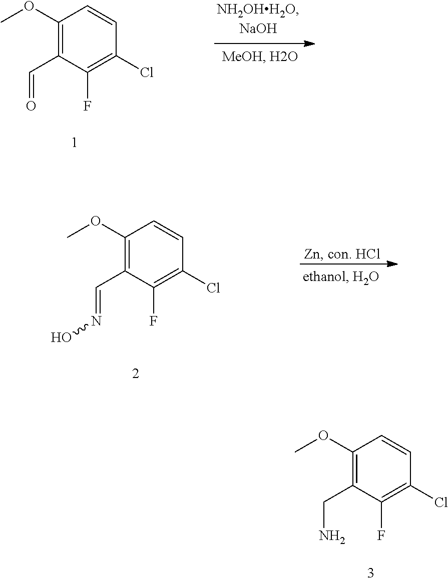

(3-chloro-2-fluoro-6-methoxyphenyl)methanamine: To a solution of 3-chloro-2-fluoro-6-methoxybenzaldehyde (2.0 g, 10.61 mmol) in ethanol (20 mL) was added NaOH (1.3 g, 32.50 mmol, 3.0 eq.) and hydroxylamine hydrochloride (2.2 g, 31.66 mmol, 1.5 eq.) at 0° C. The mixture was allowed to warm to room temperature and stirred for 3 h. water (20 mL) was added and the aqueous phase was extracted with EA (3×20 mL). The combined organic phases were washed with brine (20 mL), dried over NaSO₄, filtered and concentrated in vacuo to give the crude products, which was used directly in the next step. LC-MS: 204.1 [M+H]⁺.

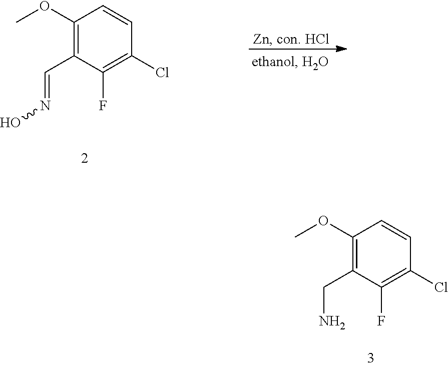

To a solution of 3-chloro-2-fluoro-6-methoxybenzaldehyde oxime (200 mg, 0.98 mmol, 1.0 eq) in ethanol (3 mL) was added con. HCl (1 mL) and water (0.5 mL) at 0° C. The mixture was stirred for 15 min. Zn powder (383 mg, 5.89 mmol, 6.0 eq) was added. The reaction mixture was heated to 80° C. for 1 h, cooled to room temperature, and filtered. The filtrate was concentrated under reduced pressure to give the hydrochloride salt of (3-chloro-2-fluoro-6-methoxyphenyl)methanamine (190 mg, 100%) as a yellow solid. LC-MS: 190.1 [M+H]⁺.

### Example 101: (R)-4-(2-(bis(methyl-d₃)amino)-2-oxoethyl)-9-chloro-N-(2-fluoro-6-methoxybenzyl)-3-methyl-5-oxo-2,3,4,5-tetrahydrobenzofuro[2,3-f][1,4]oxazepine-3-carboxamide

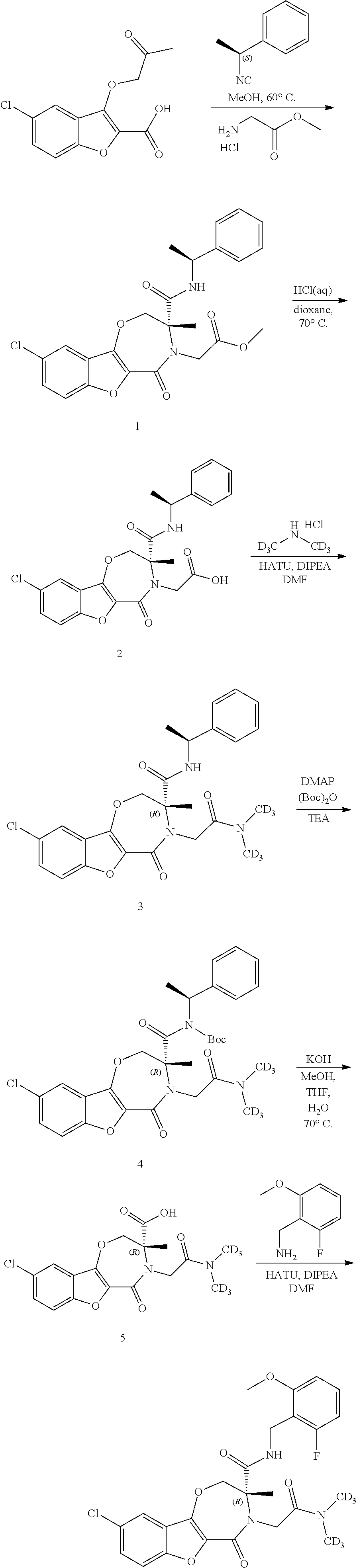

Methyl 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetate: A mixture of 5-chloro-3-(2-oxopropoxy)benzofuran-2-carboxylic acid (4 g, 14.89 mmol, 1.0 eq), methyl glycinate hydrochloride (2.8 g, 22.34 mmol, 1.5 eq), TEA (4.5 g, 44.68 mmol, 3.0 eq) and (S)-(1-isocyanoethyl)benzene (2.15 g, 16.38 mmol, 1.1 eq) in MeOH (20 mL) was stirred at 50° C. for 16 h. The mixture was concentrated under reduced pressure. The crude product was purified by flash chromatography on silica gel (PE:EA=1:1) to give the product (2 g, 28.6%) as a yellow solid. TLC: PE/EA=1:1, UV; R_{f}Starting Material=0.0, Product=0.4, isomer=0.6; LC-MS: Calculated Exact Mass=470.1, Found [M+H]⁺=471.1.

2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetic acid: A mixture of methyl 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetate (2 g, 4.2 mmol, 1.0 eq), 6 M HCl (40 mL) in dioxane (80 mL) was stirred at 80° C. for 16 h. The mixture was concentrated under reduced pressure. The solution was extracted with EA, the solvent was removed under reduced pressure to give the product (1. g, 92.8%) as a yellow solid. TLC: PE/EA=1:1, UV; R_{f}Starting Material=0.4, Product=0.0. LC-MS: Calculated Exact Mass=456.1, Found [M+H]⁺=457.1.

(R)-4-(2-(bis(methyl-d3)amino)-2-oxoethyl)-9-chloro-3-methyl-5-oxo-N—((S)-1-phenylethyl)-2,3,4,5-tetrahydrobenzofuro[2,3-f][1,4]oxazepine-3-carboxamide: To a solution of 2-((R)-9-chloro-3-methyl-5-oxo-3-(((S)-1-phenylethyl)carbamoyl)-2,3-dihydrobenzofuro[2,3-f][1,4]oxazepin-4(5H)-yl)acetic acid (1 g, 2.19 mmol, 1.0 eq) in DMF (30 mL) was added HATU (1.25 g, 3.29 mmol, 1.5 eq), bis(methyl-d3)amine hydrochloride (383 mg, 4.38 mmol, 2.0 eq) and DIPEA (1.4 g, 10.95 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (800 mg, 74.7%) as a white solid. TLC: DCM/MeOH=5:1, UV; R_{f}Starting Material=0.15, Product=0.6. LC-MS: Calculated Exact Mass=565.2, Found [M+H]⁺=566.2.

tert-butyl ((R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)((S)-1-phenylethyl)carbamate: To a solution of (R)-4-(2-(bis(methyl-d3)amino)-2-oxoethyl)-9-chloro-3-methyl-5-oxo-N—((S)-1-phenylethyl)-2,3,4,5-tetrahydrobenzofuro[2,3-f][1,4]oxazepine-3-carboxamide (750 mg, 1.53 mmol, 1.0 eq) in TEA (20 mL) and DMA (2 mL) was added (Boc)₂O (6.5 g, 30.6 mmol, 20.0 eq), DMAP (238 mg, 1.95 mmol, 1.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (750 mg, 83%) as brown solid. TLC: PE/EA=1:2, UV; R_{f}Starting Material=0.15, Product=0.6. LC-MS: Calculated Exact Mass=589.2, Found [M+H]⁺=590.2.

(R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid: To a solution of tert-butyl ((R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)((S)-1-phenylethyl)carbamate (750 mg, 1.27 mmol, 1.0 eq) in THF (5 mL), MeOH (6 mL), H₂O (3 mL) was added KOH (1.4 g, 25.4 mmol, 20.0 eq). The mixture was stirred at 70° C. for 3 hours. The reaction mixture was concentrated under reduced pressure and then acidified to pH 4-5 with 1 N HCl. The solid was filtered to give the product (410 mg, 83.6%) as white solid. The crude product used in the next step directly. LC-MS: Calculated Exact Mass=386.1, Found [M+H]⁺=387.1.

(R)-4-(2-(bis(methyl-d3)amino)-2-oxoethyl)-9-chloro-N-(2-fluoro-6-methoxybenzyl)-3-methyl-5-oxo-2,3,4,5-tetrahydrobenzofuro[2,3-f][1,4]oxazepine-3-carboxamide: To a solution of (R)-4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid (150 mg, 0.389 mmol, 1.0 eq) in DMF (2 mL) was added HATU (222 mg, 0.584 mmol, 1.5 eq). The mixture was stirred at room temperature for 10 min. (2-fluoro-6-methoxyphenyl)methanamine (72.3 mg, 0.466 mmol, 1.2 eq) and DIPEA (251 mg, 1.95 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (85 mg, 41.8%) as a white solid. LC-MS: Calculated Exact Mass=523.2, Found [M+H]⁺=524.2. ¹H NMR (400 MHz, DMSO) δ 9.55 (s, 1H), 7.58 (d, J=13.5 Hz, 1H), 7.55 (d, J=8.9 Hz, 1H), 7.49 (dd, J=8.9, 2.1 Hz, 1H), 7.04 (dd, J=15.2, 7.8 Hz, 1H), 6.61 (d, J=8.3 Hz, 1H), 6.38 (s, 1H), 4.84 (d, J=11.8 Hz, 1H), 4.49 (s, 1H), 4.38-4.22 (m, 3H), 3.92 (s, 1H), 3.63 (s, 3H), 1.53 (s, 3H).

Example 102: (R)-4-(2-(bis(methyl-d₃)amino)-2-oxoethyl)-9-chloro-N-((3-methoxypyridin-2-yl)methyl)-3-methyl-5-oxo-2,3,4,5-tetrahydrobenzofuro[2,3-f][1,4]oxazepine-3-carboxamide was synthesized using a method analogue to the synthesis of Example 101.

### Example 108: 4-(2-(bis(methyl-d₃)amino)-2-oxoethyl)-9-chloro-N-((3-ethoxypyridin-2-yl)methyl)-3-methyl-5-oxo-2,3,4,5-tetrahydrobenzofuro[2,3-f][1,4]oxazepine-3-carboxamide

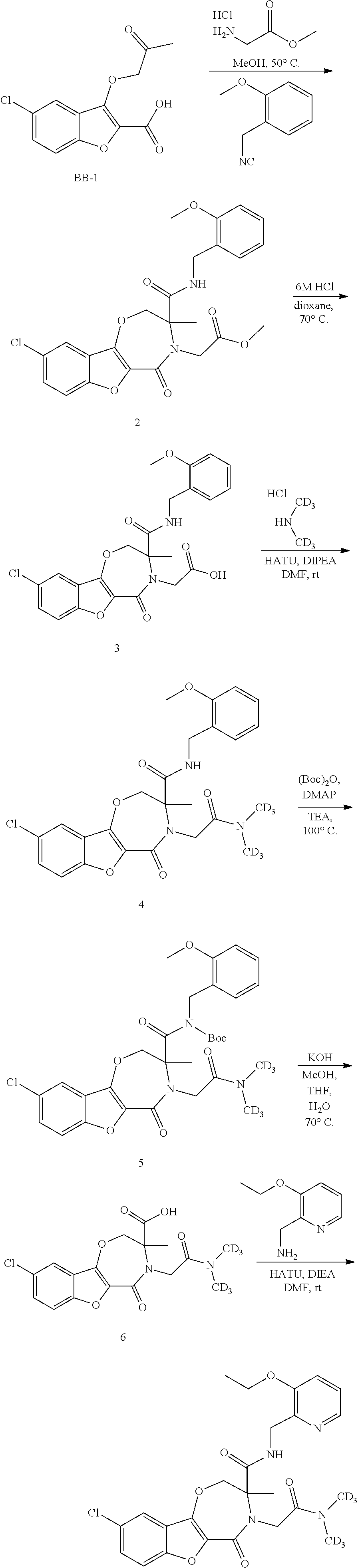

Methyl 2-(9-chloro-3-((2-methoxybenzyl)carbamoyl)-3-methyl-5-oxo-2,3-dihydrobenzofuro[2,3-f][1,4]oxazepin-4(5H)-yl)acetate: A mixture of 5-chloro-3-(2-oxopropoxy)benzofuran-2-carboxylic acid (2.4 g, 8.9 mmol, 1.0 eq), methyl glycinate hydrochloride (1.68 g, 13.4 mmol, 1.5 eq), TEA (2.71 g, 26.8 mmol, 3.0 eq) and 1-(isocyanomethyl)-2-methoxybenzene (2.63 g, 16.38 mmol, 1.2 eq) in MeOH (20 mL) was stirred at 50° C. for 16 h. The mixture was concentrated under reduced pressure. The crude product was purified by flash chromatography on silica gel (PE:EA=1:1) to give the product (2.6 g, 59.7%) as a yellow solid. TLC: PE/EA=1:1, UV; R_{f}Starting Material=0.0, Product=0.4. LC-MS: Calculated Exact Mass=486.1, Found [M+H]⁺=487.1.

2-(9-chloro-3-((2-methoxybenzyl)carbamoyl)-3-methyl-5-oxo-2,3-dihydrobenzofuro[2,3-f][1,4]oxazepin-4(5H)-yl)acetic acid: A mixture of methyl 2-(9-chloro-3-((2-methoxybenzyl)carbamoyl)-3-methyl-5-oxo-2,3-dihydrobenzofuro[2,3-f][1,4]oxazepin-4(5H)-yl)acetate (2.5 g, 5.13 mmol, 1.0 eq), 6 M HCl (40 mL) in dioxane (80 mL) was stirred at 70° C. for 16 h. The mixture was concentrated under reduced pressure. The solution was extracted with EA, the solvent was removed under reduced pressure to give the product (2.3 g, 95%) as a yellow solid. LC-MS: Calculated Exact Mass=472.0, Found [M+H]⁺=473.1.

4-(2-(bis(methyl-d3)amino)-2-oxoethyl)-9-chloro-N-(2-methoxybenzyl)-3-methyl-5-oxo-2,3,4,5-tetrahydrobenzofuro[2,3-f][1,4]oxazepine-3-carboxamide: To a solution of 2-(9-chloro-3-((2-methoxybenzyl)carbamoyl)-3-methyl-5-oxo-2,3-dihydrobenzofuro[2,3-f][1,4]oxazepin-4(5H)-yl)acetic acid (1 g, 2.1 mmol, 1.0 eq) in DMF (30 mL) was added HATU (1.2 g, 3.1 mmol, 1.5 eq), bis(methyl-d3)amine hydrochloride (370 mg, 4.2 mmol, 2.0 eq) and DIPEA (1.36 g, 10.5 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (900 mg, 85%) as a white solid. TLC: DCM/MeOH=5:1, UV; R_{f}Starting Material=0.15, Product=0.6. LC-MS: Calculated Exact Mass=505.1, Found [M+H]⁺=506.2.

tert-butyl (4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)(2-methoxybenzyl)carbamate: To a solution of 4-(2-(bis(methyl-d3)amino)-2-oxoethyl)-9-chloro-N-(2-methoxybenzyl)-3-methyl-5-oxo-2,3,4,5-tetrahydrobenzofuro[2,3-f][1,4]oxazepine-3-carboxamide (900 mg, 1.78 mmol, 1.0 eq) in TEA (20 mL) and DMA (2 mL) was added (Boc)₂O (7.7 g, 35.6 mmol, 20.0 eq), DMAP (238 mg, 1.95 mmol, 1.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (650 mg, 60%) as brown solid. TLC: PE/EA=2:1, UV; R^{f} Starting Material=0.15, Product=0.6. LC-MS: Calculated Exact Mass=605.1, Found [M+H]⁺=606.1.

4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid: To a solution of tert-butyl (4-(2-(bis(methyl-d3)amino)-2-oxoethyl)-9-chloro-3-methyl-5-oxo-2,3,4,5-tetrahydrobenzofuro[2,3-f][1,4]oxazepine-3-carbonyl)(2-methoxybenzyl)carbamatee (500 mg, 0.826 mmol, 1.0 eq) in THF (5 mL), MeOH (6 mL), H₂O (3 mL) was added KOH (0.925 g, 16.5 mmol, 20.0 eq). The mixture was stirred at 70° C. for 3 hours. The reaction mixture was concentrated under reduced pressure and then acidified to pH 4-5 with 1 N HCl. The solid was filtered to give the product (300 mg, 94%) as white solid. The crude product used in the next step directly. LC-MS: Calculated Exact Mass=386.1, Found [M+H]⁺=387.1.

4-(2-(bis(methyl-d3)amino)-2-oxoethyl)-9-chloro-N-((3-ethoxypyridin-2-yl)methyl)-3-methyl-5-oxo-2,3,4,5-tetrahydrobenzofuro[2,3-f][1,4]oxazepine-3-carboxamide: To a solution of 4-(2-(bis(methyl-d3)amino)-2-oxoethyl)-9-chloro-3-methyl-5-oxo-2,3,4,5-tetrahydrobenzofuro[2,3-f][1,4]oxazepine-3-carboxylic acid (100 mg, 0.077 mmol, 1.0 eq) in DMF (2 mL) was added HATU (141 mg, 0.116 mmol, 1.5 eq). (3-ethoxypyridin-2-yl)methanamine (43.3 mg, 0.085 mmol, 1.1 eq) and DIPEA (167 mg, 0.388 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (80 mg, 59.7%) as a white solid. LC-MS: Calculated Exact Mass=521.2, Found [M+H]⁺=522.2. ¹H NMR (400 MHz, DMSO) δ 9.42 (s, 1H), 7.63-7.61 (m, 2H), 7.52 (dd, J=8.9, 2.2 Hz, 2H), 7.18 (d, J=7.6 Hz, 1H), 7.02 (s, 1H), 4.85 (d, J=11.2 Hz, 1H), 4.48 (s, 2H), 4.40-4.23 (m, 2H), 4.21-3.80 (m, 3H), 1.58 (s, 3H), 1.27 (t, J=6.9 Hz, 3H).

Example 109: 4-(2-(bis(methyl-d₃)amino)-2-oxoethyl)-9-chloro-N-((3-isopropoxypyridin-2-yl)methyl)-3-methyl-5-oxo-2,3,4,5-tetrahydrobenzofuro[2,3-f][1,4]oxazepine-3-carboxamide was synthesized using a method analogous to the method used to synthesize Example 108.

### Example 113: (R)-4-(2-(bis(methyl-d₃)amino)-2-oxoethyl)-9-fluoro-N-(2-fluoro-6-methoxybenzyl)-3-methyl-5-oxo-2,3,4,5-tetrahydrobenzofuro[2,3-f][1,4]oxazepine-3-carboxamide

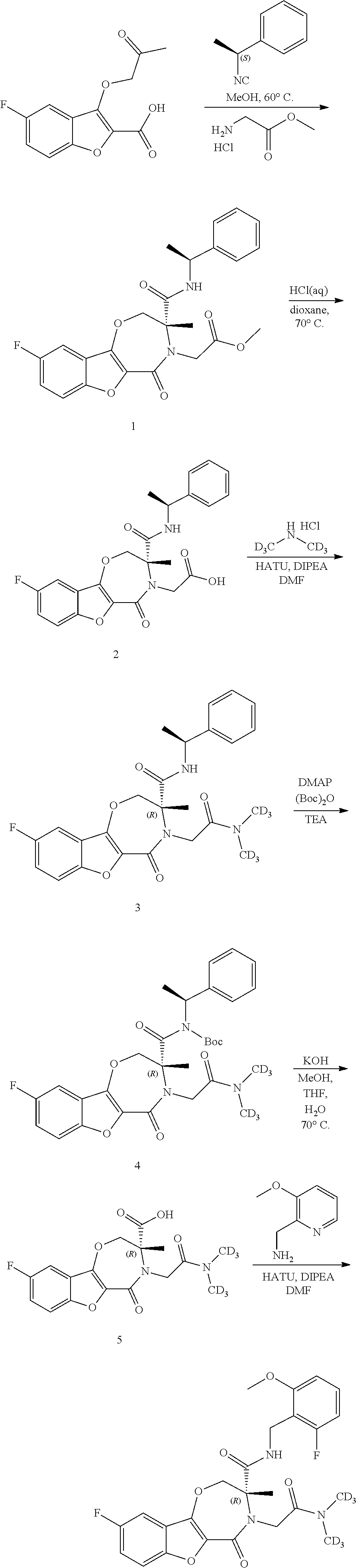

methyl 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetate: A mixture of 5-fluoro-3-(2-oxopropoxy)benzofuran-2-carboxylic acid (1.3 g, 5.155 mmol, 1.0 eq), methyl glycinate hydrochloride (1.4 g, 10.31 mmol, 2.0 eq), TEA (1.03 g, 10.31 mmol, 2.0 eq) and (S)-(1-isocyanoethyl)benzene (0.9 g, 10.31 mmol, 2.0 eq) in MeOH (20 mL) was stirred at 50° C. for 16 h. The mixture was concentrated under reduced pressure. The crude product was purified by flash chromatography on silica gel (PE:EA=1:1) to give the product (550 mg, 23.5%) as a yellow solid. TLC: PE/EA=1:1, UV; R_{f}Starting Material=0.0, Product=0.4, isomer=0.6; LC-MS: Calculated Exact Mass=454.1, Found [M+H]⁺=455.1.

2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetic acid: A mixture of methyl 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetate (500 mg, 1.1 mmol, 1.0 eq), 6 M HCl (8 mL) in dioxane (6 mL) was stirred at 70° C. for 16 h. The mixture was concentrated under reduced pressure. The solution was extracted with EA, the solvent was removed under reduced pressure to give the product (436 mg, 90%) as a yellow solid. TLC: PE/EA=1:1, UV; R_{f}Starting Material=0.4, Product=0.0. LC-MS: Calculated Exact Mass=440.1, Found [M+H]⁺=441.1.

(R)-4-(2-(bis(methyl-d3)amino)-2-oxoethyl)-9-fluoro-3-methyl-5-oxo-N—((S)-1-phenylethyl)-2,3,4,5-tetrahydrobenzofuro[2,3-f][1,4]oxazepine-3-carboxamide: To a solution of 2-((R)-9-fluoro-3-methyl-5-oxo-3-(((S)-1-phenylethyl)carbamoyl)-2,3-dihydrobenzofuro[2,3-f][1,4]oxazepin-4(5H)-yl)acetic acid (400 mg, 0.908 mmol, 1.0 eq) in DMF (30 mL) was added HATU (690 mg, 1.816 mmol, 2.0 eq), bis(methyl-d3)amine hydrochloride (92 mg, 1.816 mmol, 2.0 eq) and DIPEA (234 mg, 1.816 mmol, 2.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (200 mg, 46.7%) as a white solid. LC-MS: Calculated Exact Mass=473.2, Found [M+H]⁺=474.2.

tert-butyl ((R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carbonyl)((S)-1-phenylethyl)carbamate: To a solution of (R)-4-(2-(bis(methyl-d3)amino)-2-oxoethyl)-9-fluoro-3-methyl-5-oxo-N—((S)-1-phenylethyl)-2,3,4,5-tetrahydrobenzofuro[2,3-f][1,4]oxazepine-3-carboxamide (150 mg, 0.317 mmol, 1.0 eq) in TEA (50 mL) was added (Boc)₂O (10 g, 45.8 mmol, 144.0 eq), DMAP (18 mg, 0.158 mmol, 0.5 eq). The mixture was stirred at 100° C. for 3 hours. Water (50 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (210 mg, 100%) as brown solid. TLC: PE/EA=1:2, UV; R_{f}Starting Material=0.15, Product=0.6. LC-MS: Calculated Exact Mass=589.2, Found [M+H]⁺=590.2.

(R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carboxylic acid: To a solution of tert-butyl ((R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carbonyl)((S)-1-phenylethyl)carbamate (150 mg, 0.261 mmol, 1.0 eq) in THF (5 mL) and MeOH (10 mL) was added KOH (10%, 10 mL). The mixture was stirred at 70° C. for 3 hours. The reaction mixture was concentrated under reduced pressure and then acidified to pH 4-5 with 1 N HCl. The solid was filtered to give the product (120 mg, 89%) as white solid. The crude product used in the next step directly. LC-MS: Calculated Exact Mass=370.1, Found [M+H]⁺=371.1.

(R)-4-(2-(bis(methyl-d3)amino)-2-oxoethyl)-9-fluoro-N-((3-methoxypyridin-2-yl)methyl)-3-methyl-5-oxo-2,3,4,5-tetrahydrobenzofuro[2,3-f][1,4]oxazepine-3-carboxamide: To a solution of (R)-4-(2-(bis(methyl-d3)amino)-2-oxoethyl)-9-fluoro-3-methyl-5-oxo-2,3,4,5-tetrahydrobenzofuro[2,3-f][1,4]oxazepine-3-carboxylic acid (100 mg, 0.27 mmol, 1.0 eq) in DMF (2 mL) was added HATU (205 mg, 0.54 mmol, 2.0 eq). The mixture was stirred at room temperature for 10 min. (3-methoxypyridin-2-yl)methanamine (75 mg, 0.54 mmol, 2.0 eq) and DIPEA (70 mg, 0.54 mmol, 2.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by prep-HPLC to give the product (20 mg, 15%) as a white solid. LC-MS: Calculated Exact Mass=579 Found [M+H]⁺=580.2; ¹H NMR (400 MHz, DMSO) δ 9.41 (s, 1H), 7.62 (dd, J=9.1, 3.9 Hz, 1H), 7.56 (s, 1H), 7.41 (d, J=7.5 Hz, 1H), 7.35 (dt, J=9.2, 4.6 Hz, 1H), 7.22 (d, J=8.2 Hz, 1H), 7.06 (s, 1H), 4.84 (d, J=11.4 Hz, 1H), 4.47 (s, 2H), 4.41-4.25 (m, 2H), 4.15 (s, 1H), 3.69 (s, 3H), 1.57 (s, 3H).

Example 125: (R)-4-(2-(bis(methyl-d₃)amino)-2-oxoethyl)-N-((3-methoxypyridin-2-yl)methyl)-3-methyl-5-oxo-8-(4-(trifluoromethyl)thiazol-2-yl)-2,3,4,5-tetrahydrobenzofuro[2,3-f][1,4]oxazepine-3-carboxamide and Example 126: (R)-4-(2-(bis(methyl-d₃)amino)-2-oxoethyl)-N-((3-methoxypyridin-2-yl)methyl)-3-methyl-8-(oxazol-2-yl)-5-oxo-2,3,4,5-tetrahydrobenzofuro[2,3-f][1,4]oxazepine-3-carboxamide were synthesizing a method analogous to the method used to synthesize (R)-4-(2-(dimethylamino)-2-oxoethyl)-N-((3-methoxypyridin-2-yl)methyl)-3-methyl-8-(oxazol-2-yl)-5-oxo-2,3,4,5-tetrahydrobenzofuro[2,3-f][1,4]oxazepine-3-carboxamide

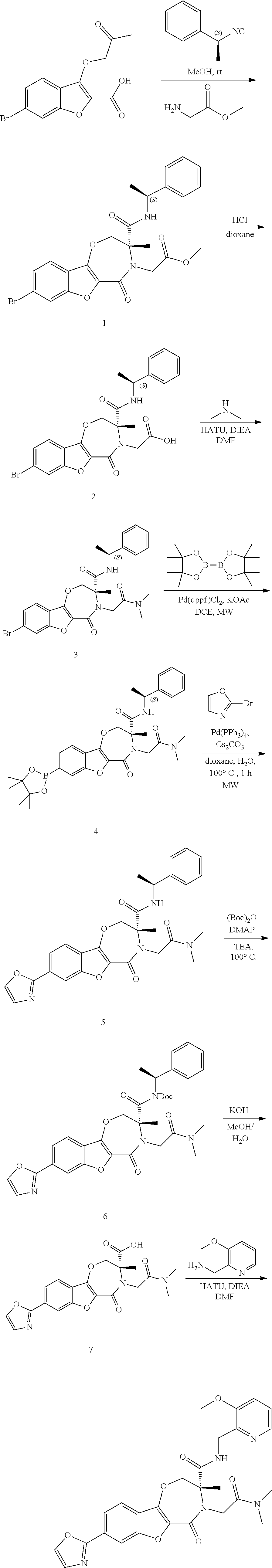

methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate: A mixture of 6-bromo-3-(2-oxopropoxy)benzofuran-2-carboxylic acid (55 g, 0.176 mol, 1.0 eq), methyl glycinate (24 g, 0.263 mol, 1.5 eq) and (S)-(1-isocyanoethyl)benzene (24 g, 0.263 mol, 1.5 eq) in MeOH (20 mL) was stirred at 50° C. for 16 h. The mixture was concentrated under reduced pressure. The crude product was purified by flash chromatography on silica gel (PE:EA=1:1) to give the product (40 g, 43.9%) as a yellow solid. TLC: PE/EA=1:1, UV; R_{f}Starting Material=0.0, Product=0.4, isomer=0.6.

2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid: A mixture of methyl 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetate (40 g, 77.62 mmol, 1.0 eq), 6M HCl (600 mL) in dioxane (800 mL) was stirred at 80° C. for 16 h. The mixture was concentrated under reduced pressure. The solution was extracted with EA, the solvent was removed under reduced pressure to give the product (35 g, 92%) as a yellow solid.

(R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide: To a solution of 2-((R)-8-bromo-3-methyl-5-oxo-3-(((S)-1-phenylethyl)carbamoyl)-2,3-dihydrobenzofuro[2,3-f][1,4]oxazepin-4(5H)-yl)acetic acid (27 g, 53.86 mmol, 1.0 eq) in DMF (30 mL) was added HATU (41 g, 107.7 mmol, 2.0 eq), dimethylamine (50 mL, 2M, 107.7 mmol, 2.0 eq) and DIPEA (45 mL, 269.3 mmol, 5.0 eq) were added. The reaction mixture was stirred at room temperature for 2 h. The reaction mixture was poured into H₂O (30 mL) and extracted with EtOAc (3×30 mL). The combined organic layers were washed with brine, dried over Na₂SO₄, filtered, and concentrated under reduced pressure. The crude product was purified by flash chromatography on silica gel (DCM:MeOH=5:1) to give the product (25 g, 89%) as a white solid.

(R)-4-(2-(dimethylamino)-2-oxoethyl)-3-methyl-5-oxo-N—((S)-1-phenylethyl)-8-(4,4,5,5-tetramethyl-1,3,2-dioxaborolan-2-yl)-2,3,4,5-tetrahydrobenzofuro[2,3-f][1,4]oxazepine-3-carboxamide: A mixture of (1,1′-bis(diphenylphosphino)ferrocene)palladium(II) chloride (3.5 g, 4.73 mmol, 0.1 eq), potassium acetate (23 g, 236.6 mmol, 5.0 eq), (R)-8-bromo-4-(2-(dimethylamino)-2-oxoethyl)-3-methyl-5-oxo-N—((S)-1-phenylethyl)-2,3,4,5-tetrahydrobenzofuro[2,3-f][1,4]oxazepine-3-carboxamide (25 g, 47.31 mmol, 1.0 eq) and bis(pinacolato)diboron (60 g, 236.6 mmol, 5.0 eq) in dioxane (600 ml) was degassed with argon for 5 min, the resulting mixture stirred at 100° C. under argon for 3 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (PE:EA=3:1) to give the product (12 g, 45%) as light-yellow solid.

(R)-4-(2-(dimethylamino)-2-oxoethyl)-3-methyl-8-(oxazol-2-yl)-5-oxo-N—((S)-1-phenylethyl)-2,3,4,5-tetrahydrobenzofuro[2,3-f][1,4]oxazepine-3-carboxamide: A mixture of Pd(PPh₃)₄ (1.2 g, 1.04 mmol, 0.1 eq), Cs₂CO₃ (6.8 g, 20.86 mmol, 2.0 eq), intermediate (R)-4-(2-(dimethylamino)-2-oxoethyl)-3-methyl-5-oxo-N—((S)-1-phenylethyl)-8-(4,4,5,5-tetramethyl-1,3,2-dioxaborolan-2-yl)-2,3,4,5-tetrahydrobenzofuro[2,3-f][1,4]oxazepine-3-carboxamide (6 g, 10.43 mmol, 2.0 eq) and 2-bromooxazole (3 g, 20.86 mmol, 2.0 eq) in dioxane (100 ml) and H₂O (10 ml) was degassed with argon for 2 mins, the resulting mixture stirred at 100° C. under argon for 16 hours. The reaction mixture was allowed to cool and diluted with ethyl acetate. This solution was washed with water. The organic layer was separated, dried over sodium sulfate and evaporated under reduced pressure, the residue was purified by flash column chromatography on silica gel (DCM:MeOH=20:1) to give the product (3.8 g, 72%) as light-yellow solid.

tert-butyl ((R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carbonyl)((S)-1-phenylethyl)carbamate: To a solution of ((R)-4-(2-(dimethylamino)-2-oxoethyl)-3-methyl-8-(oxazol-2-yl)-5-oxo-N—((S)-1-phenylethyl)-2,3,4,5-tetrahydrobenzofuro[2,3-f][1,4]oxazepine-3-carboxamide (4 g, 7.74 mmol, 1.0 eq) in TEA (100 mL) was added (Boc)₂O (17 g, 77.44 mmol, 10.0 eq), DMAP (0.95 g, 7.74 mmol, 1.0 eq). The mixture was stirred at 100° C. for 16 hours. Water (150 mL) was added. The aqueous phase was extracted with EA (3×50 mL). The combined organic solution was washed with brine (50 mL), dried over anhydrous Na₂SO₄, and the solution was concentrated under reduced pressure. The residue was purified by silica gel chromatography (PE:EA=1:2) to give the product (4 g, 75%) as brown solid.

(R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carboxylic acid: To a solution of tert-butyl ((R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carbonyl)((S)-1-phenylethyl)carbamate (3.5 g, 5.676 mmol, 1.0 eq) in THF (25 mL), MeOH (50 mL was added KOH (10%, 50 mL), the mixture was stirred at 70° C. for 3 hours. The reaction mixture was concentrated under reduced pressure and then acidified to pH 4-5 with 1 N HCl. The solid was filtered to give the product (2.3 g, 82%) as white solid. The crude product used in the next step directly.

(R)-4-(2-(dimethylamino)-2-oxoethyl)-N-((3-methoxypyridin-2-yl)methyl)-3-methyl-8-(oxazol-2-yl)-5-oxo-2,3,4,5-tetrahydrobenzofuro[2,3-f][1,4]oxazepine-3-carboxamide: To a solution of (R)-4-(2-(dimethylamino)-2-oxoethyl)-3-methyl-8-(oxazol-2-yl)-5-oxo-2,3,4,5-tetrahydrobenzofuro[2,3-f][1,4]oxazepine-3-carboxylic acid (2.3 g, 5.564 mmol, 1.0 eq) in DMF (20 mL) was added HATU (4.2 g, 11.14 mmol, 2.0 eq), (3-methoxypyridin-2-yl)methanamine (1.5 g, 11.13 mmol, 2.0 eq) and DIPEA (3.6 g, 27.82 mmol, 5.0 eq). The reaction mixture was stirred at room temperature for 2 h. The reaction mixture was poured into H₂O (130 mL) and extracted with EtOAc (3×130 mL). The combined organic layers were washed with brine, dried over Na₂SO₄, filtered, and concentrated under reduced pressure. The crude product was purified by prep-HPLC to give the product (2 g, 70%) as a white solid. LC-MS: Calculated Exact Mass=533.2, Found [M+H]⁺=534.2. ¹H NMR (400 MHz, DMSO) δ 9.45 (s, 1H), 8.30 (d, J=0.6 Hz, 1H), 8.09 (s, 1H), 7.92 (dd, J=8.3, 1.0 Hz, 1H), 7.76 (d, J=8.2 Hz, 1H), 7.55 (s, 1H), 7.46 (d, J=0.7 Hz, 1H), 7.22 (d, J=8.1 Hz, 1H), 7.02 (s, 1H), 4.88 (d, J=11.9 Hz, 1H), 4.50 (s, 2H), 4.38 (d, J=11.4 Hz, 1H), 4.31 (dd, J=15.6, 6.2 Hz, 1H), 4.14 (s, 1H), 3.69 (s, 3H), 3.06 (s, 3H), 2.86 (s, 3H), 1.59 (s, 3H).

TABLE 1

| Ex # | Structure | Analytical Data |
|---|---|---|
| 1 | 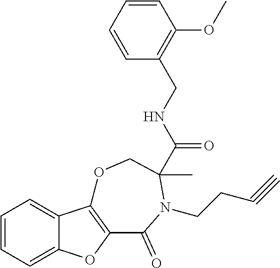 [SMILES: C#CCCN1C(=O)c2oc3ccccc3c2OCC1(C)C(=O)NCc1ccccc1OC] | ¹H NMR (400 MHz, DMSO-d₆) δ (ppm): 8.37 (s, 1H), 7.68 (ddd, J = 7.8, 1.3, 0.7 Hz, 1H), 7.60 (dt, J = 8.4, 0.9 Hz, 1H), 7.52 (ddd, J = 8.4, 7.1, 1.3 Hz, 1H), 7.34 (ddd, J = 7.9, 7.1, 0.9 Hz, 1H), 7.04 (d, J = 7.8 Hz, 1H), 6.84 (d, J = 8.1 Hz, 1H), 6.74 (s, 1H), 6.33 (s, 1H), 4.92 (d, J = 12.6 Hz, 1H), 4.40 (d, J = 12.5 Hz, 1H), 4.18 (d, J = 5.7 Hz, 2H), 3.71 (s, 5H), 3.48 (brs, 1H), 2.90 (s, 1H), 1.70 (s, 4H). |
| 2 | 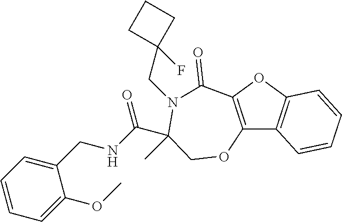 [SMILES: COc1ccccc1CNC(=O)C1(C)COc2c(oc3ccccc23)C(=O)N1CC1(F)CCC1] | 1H NMR (400 MHz, CDCl3) δ 7.50 (d, J = 7.9 Hz, 1H), 7.45 − 7.38 (m, 2H), 7.22 − 7.15 (m, 1H), 6.96 − 6.82 (m, 2H), 6.63 − 6.56 (m, 1H), 6.49 (d, J = 7.0 Hz, 2H), 4.88 (d, J = 11.8 Hz, 1H), 4.43 − 4.21 (m, 2H), 4.17 − 3.96 (m, 3H), 3.71 (s, 3H), 2.39 − 2.11 (m, 4H), 1.89 − 1.77 (m, 5H). LC-MS: 467.2[M + 1]+ |
| 3 | 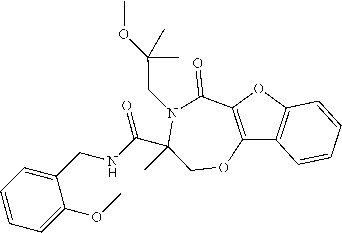 [SMILES: COc1ccccc1CNC(=O)C1(C)COc2c(oc3ccccc23)C(=O)N1CC(C)(C)OC] | 1H NMR (400 MHz, DMSO) δ 8.17 (s, 1H), 7.67 (d, J = 7.7 Hz, 1H), 7.60 (d, J = 8.4 Hz, 1H), 7.56 − 7.46 (m, 1H), 7.38 − 7.27 (m, 1H), 7.03 (t, J = 7.2 Hz, 1H), 6.82 (d, J = 8.0 Hz, 1H), 6.61 (s, 1H), 6.30 (s, 1H), 5.00 (d, J = 12.2 Hz, 1H), 4.44 (d, J = 12.2 Hz, 1H), 4.30 − 3.94 (m, 3H), 3.70 (s, 3H), 3.50 − 3.38 (m, 1H), 3.07 (s, 3H), 1.80 (s, 3H), 1.17 (s, 3H), 1.11 (s, 3H); LC-MS: 467.2[M + 1]+ |
| 4 | 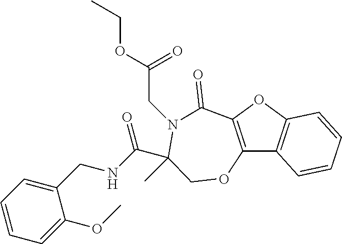 [SMILES: CCOC(=O)CN1C(=O)c2oc3ccccc3c2OCC1(C)C(=O)NCc1ccccc1OC] | 1H NMR (400 MHz, DMSO) δ 8.59 (t, J = 5.8 Hz, 1H), 7.70 (d, J = 7.8 Hz, 1H), 7.63 (d, J = 8.4 Hz, 1H), 7.59 − 7.52 (m, 1H), 7.36 (t, J = 7.5 Hz, 1H), 7.01 (t, J = 6.7 Hz, 1H), 6.82 (d, J = 8.1 Hz, 1H), 6.54 (s, 1H), 6.17 (s, 1H), 4.98 (d, J = 11.7 Hz, 1H), 4.57 (d, J = 16.4 Hz, 1H), 4.39 (d, J = 12.2 Hz, 1H), 4.31 − 4.02 (m, 5H), 3.70 (s, 3H), 1.65 (s, 3H), 1.22 (t, J = 7.1 Hz, 3H); LC-MS: 467.1[M + 1]+ |
| 5 | 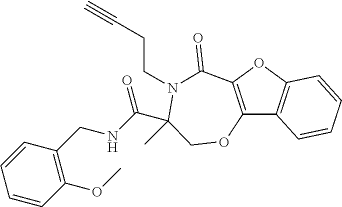 [SMILES: C#CCCN1C(=O)c2oc3ccccc3c2OCC1(C)C(=O)NCc1ccccc1OC] | 1H NMR (400 MHz, CDCl3) δ 7.52 (d, J = 7.9 Hz, 1H), 7.45 − 7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86- 3.84 (m, 1H), 3.72 (s, 3H), 3.73 − 3.58 (m, 1H), 2.73-3.69 (m, 1H), 2.66 − 2.49 (m, 1H), 2.00 (t, J = 2.7 Hz, 1H), 1.79 (s, 3H). LC-MS: 433.2[M + 1]+ |
| 6 | 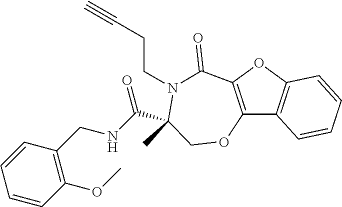 [SMILES: C#CCCN1C(=O)c2oc3ccccc3c2OC[C@]1(C)C(=O)NCc1ccccc1OC] | 1H NMR (400 MHz, CDCl3) δ 7.52 (d, J = 7.9 Hz, 1H), 7.45 − 7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86- 3.84 (m, 1H), 3.72 (s, 3H), 3.73 − 3.58 (m, 1H), 2.73-3.69 (m, 1H), 2.66 − 2.49 (m, 1H), 2.00 (t, J = 2.7 Hz, 1H), 1.79 (s, 3H). LC-MS: 433.2[M + 1]+ |
| 7 | 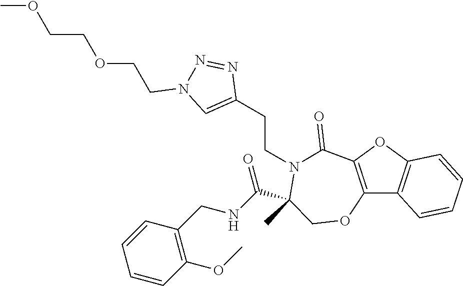 [SMILES: COCCOCCn1cc(CCN2C(=O)c3oc4ccccc4c3OC[C@]2(C)C(=O)NCc2ccccc2OC)nn1] | 1H NMR (400 MHz, CDCl3) δ 7.61 (s, 1H), 7.51 (d, J = 7.9 Hz, 1H), 7.46 − 7.37 (m, 2H), 7.20 (ddd, J = 8.0, 5.1, 3.0 Hz, 1H), 7.04 (t, J = 5.8 Hz, 1H), 6.98 (d, J = 7.2 Hz, 1H), 6.93 (t, J = 7.7 Hz, 1H), 6.54 (dd, J = 12.2, 7.8 Hz, 2H), 4.81 (d, J = 12.1 Hz, 1H), 4.62 − 4.45 (m, 2H), 4.40 (dd, J = 14.3, 6.8 Hz, 1H), 4.21 (dd, J = 14.2, 5.0 Hz, 1H), 4.08 (d, J = 12.1 Hz, 1H), 4.05 − 3.94 (m, 1H), 3.94 − 3.78 (m, 3H), 3.70 (s, 3H), 3.64 − 3.55 (m, 2H), 3.52 − 3.48 (m, 2H), 3.36 (s, 3H), 3.23-3.16 (m, 1H), 3.11 − 3.01 (m, 1H), 1.79 (s, 3H). LC-MS: 478.2[M + 1]+ |
| 8 | 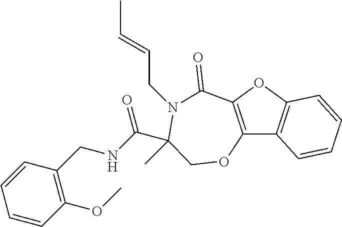 [SMILES: C/C=C/CN1C(=O)c2oc3ccccc3c2OCC1(C)C(=O)NCc1ccccc1OC] | 1H NMR (400 MHz, CDCl3) δ 7.53 (d, J = 7.9 Hz, 1H), 7.48 − 7.37 (m, 2H), 7.24-7.16 (m, 1H), 7.05-6.92 (m, 2H), 6.67 (t, J = 5.5 Hz, 1H), 6.63-6.51 (m, 2H), 5.78-5.64 (m, 1H), 5.65 − 5.49 (m, 1H), 4.78 (d, J = 11.8 Hz, 1H), 4.41 − 4.33 (m, 1H), 4.32-4.25 (m, 1H), 4.25-4.18 (m, 1H), 4.17-4.05 (m, 2H), 3.73 (s, 3H), 1.73 (s, 3H), 1.62 (dd, J = 6.1, 1.0 Hz, 3H). LC-MS: 435.20[M + 1]+ |
| 9 | 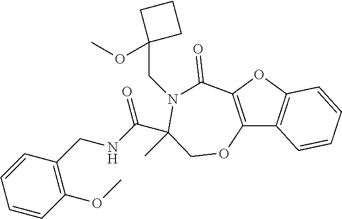 [SMILES: COc1ccccc1CNC(=O)C1(C)COc2c(oc3ccccc23)C(=O)N1CC1(OC)CCC1] | 1H NMR (400 MHz, DMSO) δ 8.20-8.05 (m, 1H), 7.66 (d, J = 7.8 Hz, 1H), 7.59 (d, J = 8.4 Hz, 1H), 7.56 − 7.45 (m, 1H), 7.33 (t, J = 7.5 Hz, 1H), 7.04 (t, J = 7.5 Hz, 1H), 6.82 (d, J = 8.1 Hz, 1H), 6.68 (s, 1H), 6.43-6.25 (m, 1H), 4.96 (d, J = 12.1 Hz, 1H), 4.35 (d, J = 12.3 Hz, 1H), 4.25 (d, J = 14.5 Hz, 1H), 4.20 − 4.03 (m, 2H), 3.74-3.71 (m, 1H), 3.70 (s, 3H), 3.10 (s, 3H), 2.31 − 1.99 (m, 2H), 1.94 − 1.79 (m, 2H), 1.75 (s, 3H), 1.72 − 1.52 (m, 2H). LC-MS: 479.20[M + 1]+ |
| 10 | 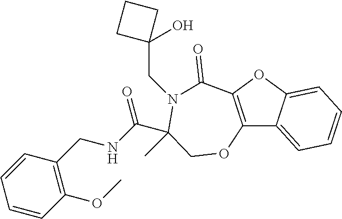 [SMILES: COc1ccccc1CNC(=O)C1(C)COc2c(oc3ccccc23)C(=O)N1CC1(O)CCC1] | 1H NMR (400 MHz, DMSO) δ 8.23 (t, J = 5.8 Hz, 1H), 7.65 (d, J = 7.8 Hz, 1H), 7.59 (d, J = 8.4 Hz, 1H), 7.57 − 7.48 (m, 1H), 7.33 (t, J = 7.2 Hz, 1H), 6.99 (t, J = 7.3 Hz, 1H), 6.80 (d, J = 8.1 Hz, 1H), 6.53 (d, J = 7.0 Hz, 1H), 6.21 (t, J = 7.2 Hz, 1H), 5.38 (s, 1H), 4.99 (d, J = 12.2 Hz, 1H), 4.45 (d, J = 12.2 Hz, 1H), 4.25 − 4.01 (m, 3H), 3.69 (s, 3H), 3.60 (d, J = 15.0 Hz, 1H), 2.25 − 2.05 (m, 2H), 1.99 − 1.84 (m, 2H), 1.82 (s, 3H), 1.74 − 1.49 (m, 2H). LC-MS: 465.20[M + 1]+ |
| 11 | 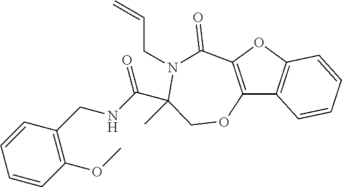 [SMILES: C=CCN1C(=O)c2oc3ccccc3c2OCC1(C)C(=O)NCc1ccccc1OC] | 1H NMR (400 MHz, CDCl3) δ 7.53 (d, J = 7.9 Hz, 1H), 7.48 − 7.38 (m, 2H), 7.24 − 7.16 (m, 1H), 7.06-6.92 (m, 2H), 6.69 (t, J = 5.4 Hz, 1H), 6.65-6.55 (m, 2H), 6.04-5.86 (m, 1H), 5.25 − 5.13 (m, 2H), 4.78 (d, J = 12.0 Hz, 1H), 4.52-4.42 (m, 1H), 4.42 − 4.33 (m, 1H), 4.28-4.20 (m, 1H), 4.19 − 4.12 (m, 1H), 4.12-4.04 (m, 1H), 3.72 (s, 3H), 1.72 (s, 3H). LC-MS: 421.20[M + 1]+ |
| 12 | 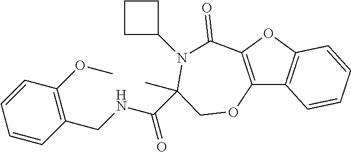 [SMILES: COc1ccccc1CNC(=O)C1(C)COc2c(oc3ccccc23)C(=O)N1C1CCC1] | 1H NMR (400 MHz, DMSO) δ 8.46 (s, 1H), 7.71 (d, J = 7.7 Hz, 1H), 7.62 (d, J = 8.4 Hz, 1H), 7.56 − 7.46 (m, 1H), 7.40 − 7.29 (m, 1H), 7.27 − 7.12 (m, 2H), 6.96 (d, J = 8.1 Hz, 1H), 6.91 − 6.78 (m, 1H), 4.62 (d, J = 12.8 Hz, 1H), 4.40 (d, J = 12.7 Hz, 1H), 4.34 − 4.19 (m, 2H), 3.81 (d, J = 14.3 Hz, 3H), 3.75 − 3.62 (m, 1H), 2.97 − 2.78 (m, 2H), 2.05 − 1.86 (m, 2H), 1.78 − 1.62 (m, 1H), 1.59 − 1.45 (m, 1H), 1.42 (s, 3H). LC- MS: 435.20[M + 1]+ |
| 13 | 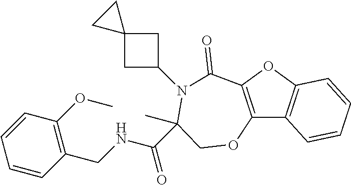 [SMILES: COc1ccccc1CNC(=O)C1(C)COc2c(oc3ccccc23)C(=O)N1C1CC2(CC2)C1] | 1H NMR (400 MHz, CDCl3) δ 7.59 (d, J = 7.8 Hz, 1H), 7.53 − 7.47 (m, 1H), 7.47 − 7.40 (m, 1H), 7.26 − 7.16 (m, 3H), 6.87 (t, J = 7.5 Hz, 1H), 6.76 (d, J = 7.9 Hz, 1H), 6.68 − 6.54 (m, 1H), 4.45 (d, J = 5.8 Hz, 2H), 4.35 (s, 2H), 4.14 − 3.96 (m, 1H), 3.74 (s, 3H), 3.22 − 3.04 (m, 2H), 2.09 − 1.95 (m, 1H), 1.91 (t, J = 9.3 Hz, 1H), 1.59 (s, 3H), 0.47 − 0.28 (m, 4H); LC-MS: 461.20[M + 1]+ |
| 14 | 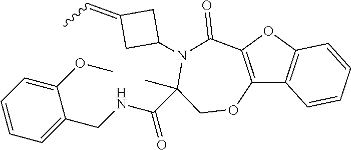 | 1H NMR (400 MHz, CDCl3) δ 7.58 (d, J = 7.9 Hz, 1H), 7.52 − 7.40 (m, 2H), 7.25 − 7.19 (m, 3H), 6.87 (t, J = 7.3 Hz, 1H), 6.77 (d, J = 7.1 Hz, 1H), 6.68 − 6.55 (m, 1H), 5.29 − 5.07 (m, 1H), 4.60 − 4.24 (m, 4H), 3.92 − 3.80 (m, 1H), 3.76 (s, 3H), 3.58 − 3.30 (m, 2H), 2.84 − 2.46 (m, 2H), 1.58 (d, J = 4.3 Hz, 3H), 1.51 − 1.41(m, 3H). LC-MS: 461.20[M + 1]+ |
| 15 | 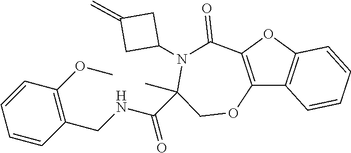 [SMILES: C=C1CC(N2C(=O)c3oc4ccccc4c3OCC2(C)C(=O)NCc2ccccc2OC)C1] | 1H NMR (400 MHz, CDCl3) δ 7.59 (d, J = 7.9 Hz, 1H), 7.52 − 7.41 (m, 2H), 7.29 − 7.26 (m, 1H), 7.25 − 7.19 (m, 2H), 6.87 (t, J = 7.4 Hz, 1H), 6.77 (d, J = 7.5 Hz, 1H), 6.56 − 6.54 (m, 1H), 4.80 (d, J = 10.5 Hz, 2H), 4.45 (d, J = 5.8 Hz, 2H), 4.41 − 4.26 (m, 2H), 3.88 − 3.79 (m, 1H), 3.76 (s, 3H), 3.68 − 3.48 (m, 2H), 2.84 − 2.68 (m, 1H), 2.68 − 2.53 (m, 1H), 1.58 (s, 3H). LC-MS: 447.20[M + 1]+ |
| 16 | 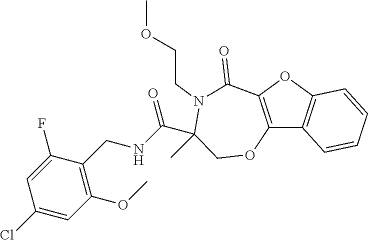 [SMILES: COCCN1C(=O)c2oc3ccccc3c2OCC1(C)C(=O)NCc1c(F)cc(Cl)cc1OC] | 1H NMR (400 MHz, CDCl3) δ 7.52 (d, J = 7.8 Hz, 1H), 7.46 − 7.33 (m, 3H), 7.20 (ddd, J = 8.0, 6.9, 1.2 Hz, 1H), 6.31-6.28 (m, 2H), 4.87 (d, J = 12.0 Hz, 1H), 4.66 (dd, J = 14.1, 7.6 Hz, 1H), 4.37-4.31 (m, 1H), 4.07 (d, J = 11.8 Hz, 1H), 3.94 − 3.86 (m, 1H), 3.79 (ddd, J = 9.8, 5.6, 4.1 Hz, 1H), 3.69 (s, 3H), 3.64 − 3.48 (m, 2H), 3.32 (s, 3H), 1.77 (s, 3H). LC-MS: 491.1[M + 1]+ |
| 17 | 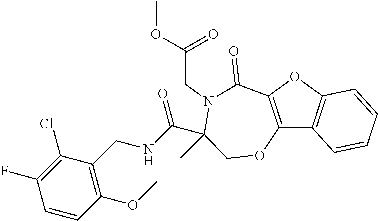 | 1H NMR (400 MHz, CDCl3) δ 7.53 (d, J = 7.9 Hz, 1H), 7.43 − 7.34 (m, 3H), 7.20 (ddd, J = 8.0, 6.3, 1.7 Hz, 1H), 6.64 (t, J = 8.7 Hz, 1H), 6.38 (dd, J = 9.0, 3.8 Hz, 1H), 4.95 − 4.75 (m, 2H), 4.36-4.30 (m, 1H), 4.19 − 4.03 (m, 2H), 3.81 − 3.75 (m, 1H), 3.70 (s, 3H), 3.62 − 3.48 (m, 2H), 3.26 (s, 3H), 1.78 (s, 3H). LC-MS: 491.1[M + 1]+ |
| 18 | 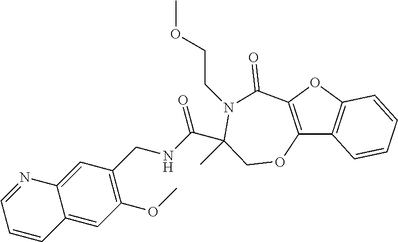 [SMILES: COCCN1C(=O)c2oc3ccccc3c2OCC1(C)C(=O)NCc1cc2ncccc2cc1OC] | 1H NMR (400 MHz, CDCl3) δ 8.66 (dd, J = 4.1, 1.3 Hz, 1H), 7.80 (d, J = 8.1 Hz, 1H), 7.72 (s, 1H), 7.66 − 7.57 (m, 1H), 7.29 − 7.16 (m, 4H), 6.87 (t, J = 7.3 Hz, 1H), 6.72 (s, 1H), 4.87 (d, J = 11.8 Hz, 1H), 4.60 (dd, J = 14.4, 7.3 Hz, 1H), 4.41 − 4.22 (m, 2H), 4.07 (d, J = 11.8 Hz, 1H), 3.88 (s, 3H), 3.85 − 3.78 (m, 1H), 3.66 − 3.53 (m, 2H), 3.30 (s, 3H), 1.81 (s, 3H); LC-MS: 490.1[M + 1]+ |
| 19 | 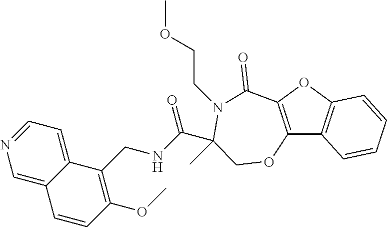 [SMILES: COCCN1C(=O)c2oc3ccccc3c2OCC1(C)C(=O)NCc1c(OC)ccc2cnccc12] | 1H NMR (400 MHz, CDCl3) δ 8.82 (s, 1H), 8.11 (d, J = 6.0 Hz, 1H), 7.71 (d, J = 6.2 Hz, 1H), 7.59 (d, J = 9.0 Hz, 1H), 7.52 − 7.45 (m, 1H), 7.41 − 7.33 (m, 1H), 7.26 − 7.18 (m, 2H), 7.16 − 7.06 (m, 2H), 4.98 (dd, J = 14.1, 7.7 Hz, 1H), 4.88 (d, J = 11.6 Hz, 1H), 4.47 − 4.25 (m, 2H), 4.02 (d, J = 11.9 Hz, 1H), 3.96 (s, 3H), 3.81 − 3.72 (m, 1H), 3.59 − 3.45 (m, 2H), 3.22 (s, 3H), 1.79 (s, 3H). LC-MS: 490.2[M + 1]+ |
| 20 | 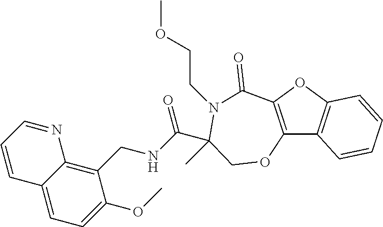 [SMILES: COCCN1C(=O)c2oc3ccccc3c2OCC1(C)C(=O)NCc1c(OC)ccc2cccnc12] | 1H NMR (400 MHz, CDCl3) δ 8.83 (d, J = 2.8 Hz, 1H), 8.48 (s, 1H), 7.93 (d, J = 7.0 Hz, 1H), 7.46 (d, J = 8.9 Hz, 1H), 7.42 − 7.30 (m, 2H), 7.26-7.22 (m, 2H), 7.11 (t, J = 7.5 Hz, 1H), 7.02 (d, J = 8.7 Hz, 1H), 5.22 (dd, J = 14.3, 7.0 Hz, 1H), 4.81 (d, J = 12.2 Hz, 1H), 4.69 (d, J = 11.3 Hz, 1H), 4.07 (d, J = 11.8 Hz, 1H), 3.97 − 3.86 (m, 1H), 3.86 − 3.73 (m, 4H), 3.71 − 3.56 (m, 2H), 3.23 (s, 3H), 1.77 (s, 3H); LC-MS: 490.1[M + 1]+ |
| 21 | 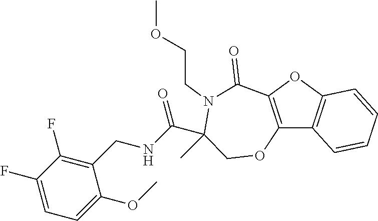 [SMILES: COCCN1C(=O)c2oc3ccccc3c2OCC1(C)C(=O)NCc1c(OC)ccc(F)c1F] | 1H NMR (400 MHz, CDCl3) δ 7.54 (d, J = 7.9 Hz, 1H), 7.47 − 7.36 (m, 3H), 7.20 (ddd, J = 8.0, 5.9, 2.1 Hz, 1H), 6.61 (dd, J = 18.5, 9.3 Hz, 1H), 6.16 (d, J = 8.6 Hz, 1H), 4.87 (d, J = 11.8 Hz, 1H), 4.66 (dd, J = 14.0, 7.2 Hz, 1H), 4.41 − 4.27 (m, 1H), 4.15 − 3.95 (m, 2H), 3.84 − 3.75 (m, 1H), 3.67 (s, 3H), 3.63 − 3.47 (m, 2H), 3.30 (s, 3H), 1.77 (s, 3H). LC-MS: 475.1[M + 1]+ |
| 22 | 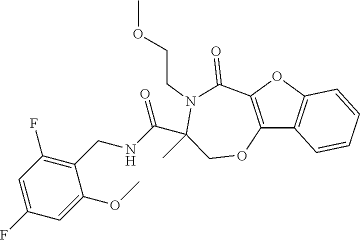 [SMILES: COCCN1C(=O)c2oc3ccccc3c2OCC1(C)C(=O)NCc1c(F)cc(F)cc1OC] | 1H NMR (400 MHz, CDCl3) δ 7.53 (d, J = 7.9 Hz, 1H), 7.45 − 7.32 (m, 3H), 7.19 (ddd, J = 8.0, 6.6, 1.5 Hz, 1H), 6.12 − 5.97 (m, 2H), 4.87 (d, J = 11.8 Hz, 1H), 4.63 (dd, J = 14.1, 7.5 Hz, 1H), 4.44 − 4.26 (m, 1H), 4.07 (d, J = 11.9 Hz, 1H), 3.91 (d, J = 11.2 Hz, 1H), 3.84 − 3.76 (m, 1H), 3.68 (s, 3H), 3.63 − 3.48 (m, 2H), 3.31 (s, 3H), 1.77 (s, 3H). LC-MS: 475.1[M + 1]+ |
| 23 | 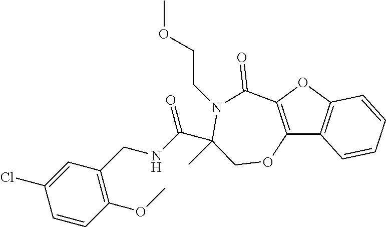 [SMILES: COCCN1C(=O)c2oc3ccccc3c2OCC1(C)C(=O)NCc1cc(Cl)ccc1OC] | 1H NMR (400 MHz, CDCl3) δ 7.57 − 7.47 (m, 2H), 7.45 − 7.35 (m, 2H), 7.20 (ddd, J = 8.0, 6.4, 1.7 Hz, 1H), 6.95 (d, J = 2.5 Hz, 1H), 6.74 (d, J = 6.8 Hz, 1H), 6.41 (d, J = 8.7 Hz, 1H), 4.88 (d, J = 11.8 Hz, 1H), 4.43 − 4.28 (m, 2H), 4.11 − 3.99 (m, 2H), 3.84 − 3.77 (m, 1H), 3.74 (s, 3H), 3.65 − 3.50 (m, 2H), 3.31 (s, 3H), 1.78 (s, 3H). LC-MS: 473.1[M + 1]+ |
| 24 | 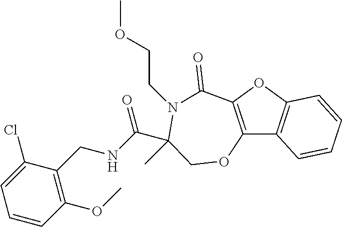 [SMILES: COCCN1C(=O)c2oc3ccccc3c2OCC1(C)C(=O)NCc1c(Cl)cccc1OC] | 1H NMR (400 MHz, CDCl3) δ 7.55 (d, J = 7.9 Hz, 1H), 7.42 − 7.37 (m, 2H), 7.26 − 7.23 (m, 1H), 7.22 − 7.16 (m, 1H), 6.85 (t, J = 8.1 Hz, 1H), 6.67 (d, J = 8.0 Hz, 1H), 6.49 (d, J = 8.2 Hz, 1H), 4.85 (d, J = 11.9 Hz, 1H), 4.73 (dd, J = 13.8, 6.6 Hz, 1H), 4.29 − 4.19 (m, 2H), 4.10 (d, J = 11.9 Hz, 1H), 3.81 − 3.67 (m, 4H), 3.61 − 3.50 (m, 2H), 3.19 (s, 3H), 1.76 (s, 3H); LC-MS: 473.1[M + 1]+ |
| 25 | 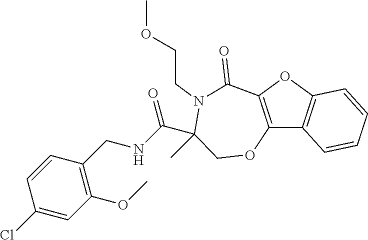 [SMILES: COCCN1C(=O)c2oc3ccccc3c2OCC1(C)C(=O)NCc1ccc(Cl)cc1OC] | 1H NMR (400 MHz, CDCl3) δ 7.54 − 7.36 (m, 4H), 7.20 (ddd, J = 8.0, 6.9, 1.2 Hz, 1H), 6.75 (d, J = 8.0 Hz, 1H), 6.50 (d, J = 1.4 Hz, 1H), 6.34 (d, J = 7.6 Hz, 1H), 4.88 (d, J = 11.8 Hz, 1H), 4.46 − 4.26 (m, 2H), 4.08 (d, J = 11.9 Hz, 1H), 3.98 (dd, J = 14.3, 4.6 Hz, 1H), 3.84 − 3.71 (m, 4H), 3.65 − 3.51 (m, 2H), 3.32 (s, 3H), 1.78 (s, 3H). LC-MS: 473.1[M + 1]+ |
| 26 | 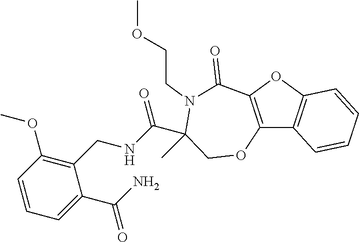 [SMILES: COCCN1C(=O)c2oc3ccccc3c2OCC1(C)C(=O)NCc1c(OC)cccc1C(N)=O] | 1H NMR (400 MHz, CDCl3) δ 8.61 (s, 1H), 8.03 (s, 1H), 7.42 − 7.32 (m, 3H), 7.12 (ddd, J = 8.1, 6.6, 1.4 Hz, 1H), 6.80 − 6.69 (m, 2H), 6.52 − 6.42 (m, 1H), 5.94 (s, 1H), 4.88 − 4.75 (m, 2H), 4.45 − 4.32 (m, 1H), 4.08 − 3.96 (m, 2H), 3.88 − 3.79 (m, 4H), 3.70 − 3.55 (m, 2H), 3.45 (s, 3H), 1.78 (s, 3H); LC-MS: 482.2[M + 1]+ |
| 27 | 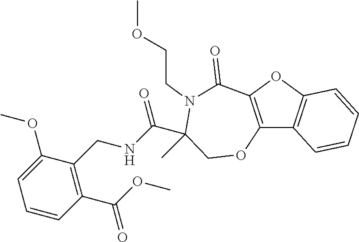 [SMILES: COCCN1C(=O)c2oc3ccccc3c2OCC1(C)C(=O)NCc1c(OC)cccc1C(=O)OC] | 1H NMR (400 MHz, CDCl3) δ 7.48 (d, J = 7.8 Hz, 1H), 7.46 − 7.34 (m, 3H), 7.20 − 7.15 (m, 1H), 7.12 (d, J = 7.7 Hz, 1H), 6.92 (s, 1H), 6.68 (d, J = 7.5 Hz, 1H), 4.99 (dd, J = 13.6, 7.3 Hz, 1H), 4.80 (d, J = 11.2 Hz, 1H), 4.24 (d, J = 10.6 Hz, 1H), 4.08 (d, J = 11.6 Hz, 2H), 3.89 (s, 3H), 3.75 − 3.66 (m, 5H), 3.63 − 3.56 (m, 1H), 3.26 (s, 3H), 1.76 (s, 3H). LC-MS: 497.2[M + 1]+ |
| 28 | 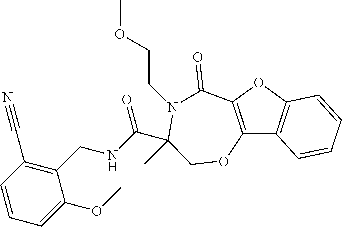 [SMILES: COCCN1C(=O)c2oc3ccccc3c2OCC1(C)C(=O)NCc1c(C#N)cccc1OC] | 1H NMR (400 MHz, CDCl3) δ 7.59 (s, 1H), 7.48 (d, J = 7.9 Hz, 1H), 7.43 − 7.35 (m, 2H), 7.21 − 7.14 (m, 1H), 6.85 (d, J = 4.1 Hz, 2H), 6.70 − 6.57 (m, 1H), 4.87 (d, J = 11.8 Hz, 1H), 4.80 (dd, J = 14.1, 7.2 Hz, 1H), 4.43 − 4.31 (m, 1H), 4.13 (dd, J = 14.0, 4.2 Hz, 1H), 4.05 (d, J = 11.8 Hz, 1H), 3.85 − 3.78 (m, 1H), 3.71 (s, 3H), 3.65 − 3.58 (m, 1H), 3.57 − 3.47 (m, 1H), 3.33 (s, 3H), 1.78 (s, 3H); LC-MS: 464.2[M + 1]+ |
| 29 | 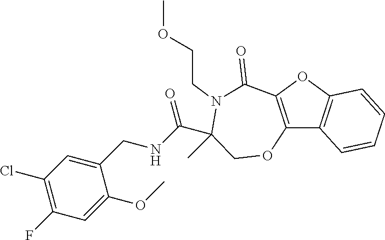 [SMILES: COCCN1C(=O)c2oc3ccccc3c2OCC1(C)C(=O)NCc1cc(Cl)c(F)cc1OC] | 1H NMR (400 MHz, CDCl3) δ 7.56 (s, 1H), 7.49 (d, J = 7.8 Hz, 1H), 7.42 (ddd, J = 8.4, 7.1, 1.3 Hz, 1H), 7.36 (d, J = 8.4 Hz, 1H), 7.23 − 7.15 (m, 1H), 6.96 (d, J = 8.3 Hz, 1H), 6.26 (d, J = 10.7 Hz, 1H), 4.88 (d, J = 11.9 Hz, 1H), 4.50 − 4.30 (m, 2H), 4.06 (d, J = 11.8 Hz, 1H), 3.92 (dd, J = 14.2, 4.4 Hz, 1H), 3.85 − 3.74 (m, 4H), 3.66 − 3.58 (m, 1H), 3.56 − 3.48 (m, 1H), 3.37 (s, 3H), 1.79 (s, 3H); LC-MS: 491.2[M + 1]+ |
| 30 | 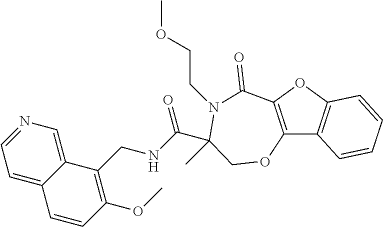 [SMILES: COCCN1C(=O)c2oc3ccccc3c2OCC1(C)C(=O)NCc1c(OC)ccc2ccncc12] | 1H NMR (400 MHz, CDCl3) δ 9.48 (s, 1H), 8.29 (d, J = 5.6 Hz, 1H), 7.48 (d, J = 9.0 Hz, 2H), 7.39 − 7.28 (m, 4H), 7.20 (d, J = 9.0 Hz, 1H), 7.10 (t, J = 7.3 Hz, 1H), 5.07 (dd, J = 14.1, 6.8 Hz, 1H), 4.85 (d, J = 11.5 Hz, 1H), 4.55 (dd, J = 14.0, 4.5 Hz, 1H), 4.28 (s, 1H), 4.05 (d, J = 11.8 Hz, 1H), 3.88 (s, 3H), 3.80 − 3.71 (m, 1H), 3.56 − 3.44 (m, 2H), 3.09 (s, 3H), 1.77 (s, 3H). LC-MS: 490.2[M + 1]+ |
| 31 | 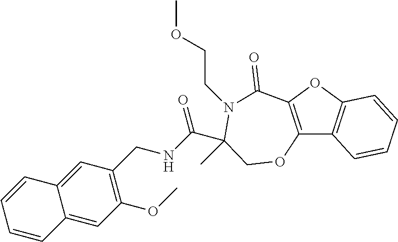 [SMILES: COCCN1C(=O)c2oc3ccccc3c2OCC1(C)C(=O)NCc1cc2ccccc2cc1OC] | 1H NMR (400 MHz, CDCl3) δ 7.55 (t, J = 12.0, 6.8 Hz, 1H), 7.48 (d, J = 8.1 Hz, 1H), 7.42 − 7.34 (m, 2H), 7.30 (ddd, J = 8.5, 6.2, 2.7 Hz, 2H), 7.25 − 7.13 (m, 3H), 6.87 (t, J = 7.3 Hz, 1H), 6.79 (s, 1H), 4.87 (d, J = 11.8 Hz, 1H), 4.58 (dd, J = 14.3, 7.5 Hz, 1H), 4.32-4.30 (m, 1H), 4.21 (dd, J = 14.3, 4.5 Hz, 1H), 4.07 (d, J = 11.8 Hz, 1H), 3.92 (s, 3H), 3.85 − 3.72 (m, 1H), 3.65 − 3.52 (m, 2H), 3.38 − 3.11 (m, 3H), 1.79 (s, 3H); LC-MS: 489.2[M + 1]+ |
| 32 | 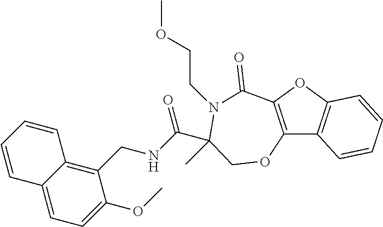 [SMILES: COCCN1C(=O)c2oc3ccccc3c2OCC1(C)C(=O)NCc1c(OC)ccc2ccccc12] | 1H NMR (400 MHz, CDCl3) δ 7.86 − 7.76 (m, 1H), 7.59 (dd, J = 8.9, 4.6 Hz, 2H), 7.45 (d, J = 7.8 Hz, 1H), 7.39 (ddd, J = 8.3, 7.0, 1.3 Hz, 1H), 7.33 (d, J = 8.4 Hz, 1H), 7.30 (s, 1H), 7.22 − 7.11 (m, 3H), 7.08 (d, J = 9.0 Hz, 1H), 4.87 (dd, J = 14.0, 6.2 Hz, 2H), 4.67 (dd, J = 14.0, 4.4 Hz, 1H), 4.24 − 4.00 (m, 2H), 3.88 (s, 3H), 3.70-3.67 (m, 1H), 3.57 − 3.37 (m, 2H), 2.96 (s, 3H), 1.75 (s, 3H); LC-MS: 489.2[M + 1]+ |
| 33 | 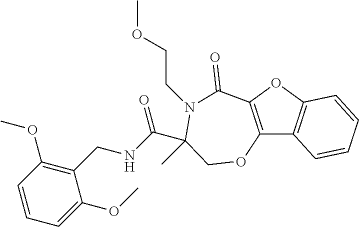 [SMILES: COCCN1C(=O)c2oc3ccccc3c2OCC1(C)C(=O)NCc1c(OC)cccc1OC] | 1H NMR (400 MHz, CDCl3) δ 7.55 (d, J = 7.9 Hz, 1H), 7.39 (d, J = 3.6 Hz, 2H), 7.19 (dt, J = 8.0, 4.0 Hz, 1H), 7.11 (s, 1H), 6.92 (t, J = 8.3 Hz, 1H), 6.28 (d, J = 8.3 Hz, 2H), 4.83 (d, J = 11.7 Hz, 1H), 4.59 (dd, J = 13.8, 6.5 Hz, 1H), 4.27 − 4.02 (m, 3H), 3.69 (s, 6H), 3.67 − 3.50 (m, 3H), 3.19 (s, 3H), 1.75 (s, 3H). LC-MS: 469.2[M + 1]+ |
| 34 | 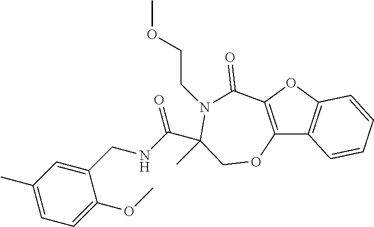 [SMILES: COCCN1C(=O)c2oc3ccccc3c2OCC1(C)C(=O)NCc1cc(C)ccc1OC] | 1H NMR (400 MHz, CDCl3) δ 7.52 (d, J = 7.9 Hz, 1H), 7.44 − 7.32 (m, 3H), 7.20-7.16 (m , 1H), 6.74 (s, 1H), 6.65 (d, J = 7.9 Hz, 1H), 6.45 (d, J = 8.2 Hz, 1H), 4.86 (d, J = 11.9 Hz, 1H), 4.34 (dd, J = 14.1, 7.1 Hz, 1H), 4.28 − 3.96 (m, 3H), 3.79-3.78 (m, 1H), 3.73 (s, 3H), 3.59-3.57 (m, 2H), 3.26 (s, 3H), 1.97 (s, 3H), 1.77 (s, 3H). LC-MS: 453.2[M + 1]+ |
| 35 | 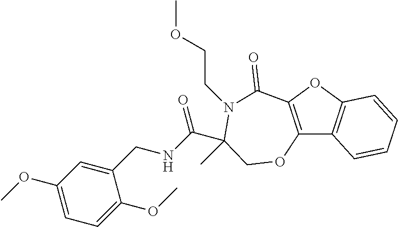 [SMILES: COCCN1C(=O)c2oc3ccccc3c2OCC1(C)C(=O)NCc1cc(OC)ccc1OC] | 1H NMR (400 MHz, CDCl3) δ 7.54 (d, J = 7.9 Hz, 1H), 7.46 − 7.33 (m, 3H), 7.21-7.16 (m, 1H), 6.57 (d, J = 2.8 Hz, 1H), 6.50 (d, J = 8.8 Hz, 1H), 6.42 (dd, J = 8.8, 2.5 Hz, 1H), 4.86 (d, J = 11.8 Hz, 1H), 4.31 (dd, J = 14.2, 6.9 Hz, 1H), 4.26 − 4.01 (m, 3H), 3.86 − 3.74 (m, 1H), 3.72 (s, 3H), 3.65 − 3.58 (m, 2H), 3.56 (s, 3H), 3.25 (s, 3H), 1.77 (s, 3H). LC-MS: 469.0[M + 1]+ |
| 36 | 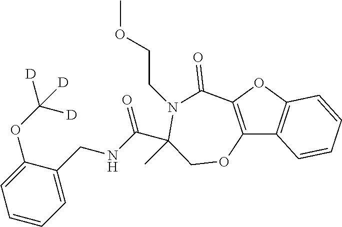 [SMILES: [2H]C([2H])([2H])Oc1ccccc1CNC(=O)C1(C)COc2c(oc3ccccc23)C(=O)N1CCOC] | 1H NMR (400 MHz, CDCl3) δ 7.54 (d, J = 7.9 Hz, 1H), 7.41-7.40 (m, 3H), 7.20-7.18 (m, 1H), 6.88 (d, J = 6.9 Hz, 2H), 6.55 (d, J = 7.9 Hz, 1H), 6.46 ((t, J = 4 Hz, 1H), 4.87 (d, J = 12.0 Hz, 1H), 4.46 − 4.18 (m, 2H), 4.11 (t, J = 13.3 Hz, 2H), 3.77-3.75 (m, 1H), 3.60-3.55 (m, 2H), 3.24 (s, 3H), 1.77 (s, 3H). LC-MS: 442.2[M + 1]+ |
| 37 | 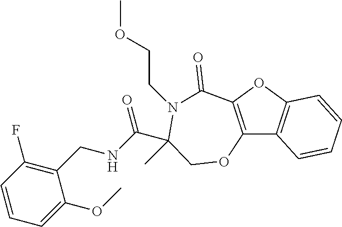 [SMILES: COCCN1C(=O)c2oc3ccccc3c2OCC1(C)C(=O)NCc1c(F)cccc1OC] | 1H NMR (400 MHz, CDCl3) δ 7.54-7.51 (m, 2H), 7.45 − 7.34 (m, 2H), 7.22-7.18 (m, 1H), 6.67 (dd, J = 8.4, 2.8 Hz, 1H), 6.57 − 6.35 (m, 2H), 4.88 (d, J = 11.9 Hz, 1H), 4.37-4.31 (m, 2H), 4.18 − 3.94 (m, 2H), 3.89 − 3.76 (m, 1H), 3.72 (s, 3H), 3.65 − 3.45 (m, 2H), 3.30 (s, 3H), 1.78 (s, 3H). LC- MS: 457.2[M + 1]+ |
| 38 | 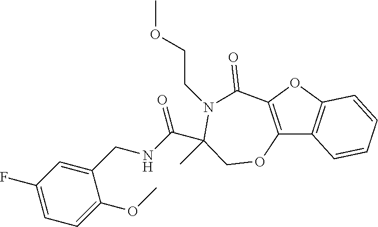 [SMILES: COCCN1C(=O)c2oc3ccccc3c2OCC1(C)C(=O)NCc1cc(F)ccc1OC] | 1H NMR (400 MHz, CDCl3) δ 7.54-7.51 (m, 2H), 7.45 − 7.32 (m, 2H), 7.19 (ddd, J = 8.0, 5.5, 2.5 Hz, 1H), 6.67 (dd, J = 8.4, 2.8 Hz, 1H), 6.50 (dt, J = 8.3, 4.3 Hz, 1H), 6.43 (dd, J = 8.8, 4.3 Hz, 1H), 4.88 (d, J = 11.8 Hz, 1H), 4.37-4.31 (m, 2H), 4.17 − 4.00 (m, 2H), 3.87 − 3.76 (m, 1H), 3.72 (s, 3H), 3.64 − 3.49 (m, 2H), 3.30 (s, 3H), 1.78 (s, 3H). LC-MS: 457.2[M + 1]+ |
| 39 | 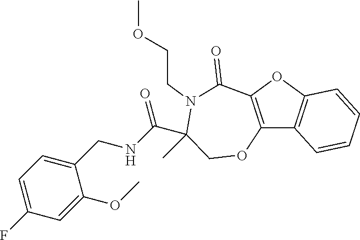 [SMILES: COCCN1C(=O)c2oc3ccccc3c2OCC1(C)C(=O)NCc1ccc(F)cc1OC] | 1H NMR (400 MHz, CDCl3) δ 7.51 (d, J = 7.9 Hz, 1H), 7.45-7.38 (m, 3H), 7.19 (ddd, J = 8.0, 6.6, 1.5 Hz, 1H), 6.79 (t, J = 7.4 Hz, 1H), 6.24 (dd, J = 10.8, 2.0 Hz, 1H), 6.05 (t, J = 7.4 Hz, 1H), 4.88 (d, J = 11.8 Hz, 1H), 4.40-4.34 (m, 2H), 4.08 (d, J = 11.9 Hz, 1H), 3.99 (dd, J = 14.2, 4.5 Hz, 1H), 3.78 (ddd, J = 7.5, 5.8, 3.1 Hz, 1H), 3.74 (s, 3H), 3.62-3.55 (m, 2H), 3.31 (s, 3H), 1.78 (s, 3H). LC-MS: 457.2[M + 1]+ |
| 40 | 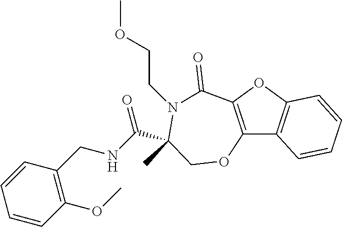 [SMILES: COCCN1C(=O)c2oc3ccccc3c2OC[C@]1(C)C(=O)NCc1ccccc1OC] | 1H NMR (400 MHz, CDCl3) δ 7.54 (d, J = 7.9 Hz, 1H), 7.47 − 7.33 (m, 3H), 7.23 − 7.14 (m, 1H), 6.91 − 6.87 (m, 2H), 6.56 (d, J = 8.0 Hz, 1H), 6.46 (t, J = 7.2 Hz, 1H), 4.87 (d, J = 11.8 Hz, 1H), 4.37 (dd, J = 14.3, 6.8 Hz, 1H), 4.30 − 4.18 (m, 1H), 4.18 − 4.00 (m, 2H), 3.85 − 3.74 (m, 1H), 3.74 (s, 3H), 3.65 − 3.51 (m, 2H), 3.24 (s, 3H), 1.77 (s, 3H). LC-MS: 438.99[M + 1]+ |
| 41 | 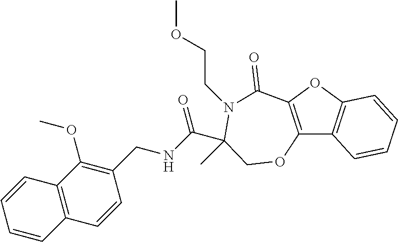 [SMILES: COCCN1C(=O)c2oc3ccccc3c2OCC1(C)C(=O)NCc1ccc2ccccc2c1OC] | 1H NMR (400 MHz, CDCl3) δ 7.96 (d, J = 8.1 Hz, 1H), 7.65 (d, J = 7.5 Hz, 2H), 7.55 (t, J = 8.0 Hz, 2H), 7.48 − 7.32 (m, 4H), 7.25 (t, J = 8.0 Hz, 2H), 7.05 (d, J = 8.0 Hz, 1H), 6.91 (d, J = 8.3 Hz, 1H), 4.94 (d, J = 11.5 Hz, 1H), 4.60 (dd, J = 14.6, 6.3 Hz, 1H), 4.40 (dd, J = 14.6, 5.2 Hz, 1H), 4.32 − 4.21 (m, 1H), 4.18 (d, J = 11.9 Hz, 1H), 3.99 − 3.81 (m, 1H), 3.81 (s, 3H), 3.58 − 3.49 (m, 2H), 3.13 (s, 3H), 1.80 (s, 3H). LC- MS: 489.2[M + 1]+ |
| 42 | 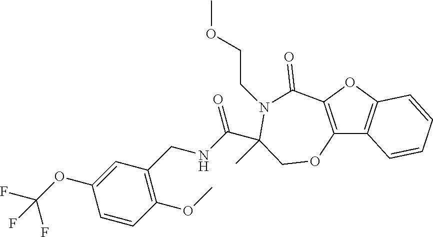 [SMILES: COCCN1C(=O)c2oc3ccccc3c2OCC1(C)C(=O)NCc1cc(OC(F)(F)F)ccc1OC] | 1H NMR (400 MHz, CDCl3) δ 7.55 (t, J = 5.4 Hz, 1H), 7.50 (d, J = 7.9 Hz, 1H), 7.45 − 7.32 (m, 2H), 7.18 (ddd, J = 8.0, 5.0, 3.0 Hz, 1H), 6.90 (d, J = 2.2 Hz, 1H), 6.71 (d, J = 8.0 Hz, 1H), 6.47 (d, J = 8.9 Hz, 1H), 4.87 (d, J = 11.9 Hz, 1H), 4.33 (dd, J = 14.4, 7.0 Hz, 2H), 4.19 − 3.98 (m, 2H), 3.83 − 3.78 (m, 1H), 3.75 (s, 3H), 3.62 − 3.51 (m, 2H), 3.28 (s, 3H), 1.77 (s, 3H). LC-MS: 523.1[M + 1]+ |
| 43 | 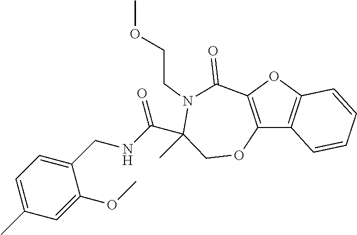 [SMILES: COCCN1C(=O)c2oc3ccccc3c2OCC1(C)C(=O)NCc1ccc(C)cc1OC] | 1H NMR (400 MHz, CDCl3) δ 7.53 (d, J = 7.9 Hz, 1H), 7.45 − 7.37 (m, 2H), 7.34 (s, 1H), 7.18 (ddd, J = 8.0, 4.7, 3.4 Hz, 1H), 6.78 (d, J = 7.5 Hz, 1H), 6.36 (s, 1H), 6.28 (d, J = 7.3 Hz, 1H), 4.86 (d, J = 12.0 Hz, 1H), 4.35 (dd, J = 14.2, 7.0 Hz, 1H), 4.25 − 4.21 (m, 1H), 4.11 − 4.06 (m, 2H), 3.81 − 3.65 (m, 4H), 3.62 − 3.56 (m, 2H), 3.26 (s, 3H), 2.12 (s, 3H), 1.77 (s, 3H). LC-MS: 453.0[M + 1]+ |
| 44 | 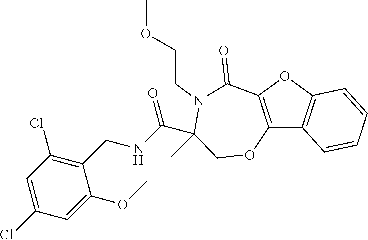 [SMILES: COCCN1C(=O)c2oc3ccccc3c2OCC1(C)C(=O)NCc1c(Cl)cc(Cl)cc1OC] | 1H NMR (400 MHz, CDCl3) δ 7.53 (d, J = 7.8 Hz, 1H), 7.46 − 7.37 (m, 2H), 7.34 (s, 1H), 7.20 (ddd, J = 8.0, 6.9, 1.1 Hz, 1H), 6.59 (s, 1H), 6.43 (s, 1H), 4.98 − 4.73 (m, 2H), 4.35 − 4.31 (m, 1H), 4.08 (d, J = 11.8 Hz, 1H), 4.04 − 3.91 (m, 1H), 3.85 − 3.74 (m, 1H), 3.71 (s, 3H), 3.65 − 3.45 (m, 2H), 3.29 (s, 3H), 1.78 (s, 3H). LC-MS: 507.0[M + 1]+ |
| 45 | 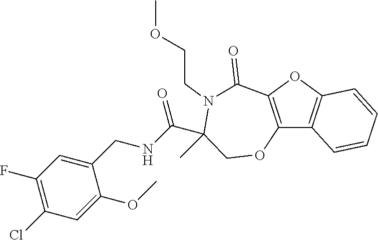 [SMILES: COCCN1C(=O)c2oc3ccccc3c2OCC1(C)C(=O)NCc1cc(F)c(Cl)cc1OC] | 1H NMR (400 MHz, CDCl3) δ 7.55 (t, J=5.6 Hz, 1H), 7.50 (d, J = 7.9 Hz, 1H), 7.48 − 7.41 (m, 1H), 7.38 (d, J = 8.3 Hz, 1H), 7.20 (t, J = 7.4 Hz, 1H), 6.71 (d, J = 8.9 Hz, 1H), 6.48 (d, J = 5.9 Hz, 1H), 4.89 (d, J = 11.9 Hz, 1H), 4.47 − 4.28 (m, 2H), 4.08 (d, J = 11.9 Hz, 1H), 3.95 (dd, J = 14.4, 4.7 Hz, 1H), 3.87 − 3.78 (m, 1H), 3.73 (s, 3H), 3.68 − 3.49 (m, 2H), 3.36 (s, 3H), 1.79 (s, 3H). LC-MS: 491.10[M + 1]+ |
| 46 | 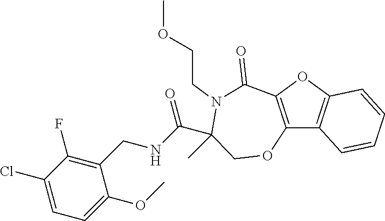 [SMILES: COCCN1C(=O)c2oc3ccccc3c2OCC1(C)C(=O)NCc1c(OC)ccc(Cl)c1F] | 1H NMR (400 MHz, CDCl3) δ 7.53 (d, J = 7.8 Hz, 1H), 7.49 − 7.35 (m, 3H), 7.20 (ddd, J = 8.0, 6.2, 1.9 Hz, 1H), 6.82 (t, J = 8.6 Hz, 1H), 6.23 (d, J = 8.8 Hz, 1H), 4.86 (d, J = 11.8 Hz, 1H), 4.68 (dd, J = 14.0, 7.3 Hz, 1H), 4.42 − 4.26 (m, 1H), 4.14 − 3.93 (m, 2H), 3.86 − 3.74 (m, 1H), 3.69 (s, 3H), 3.65 − 3.46 (m, 2H), 3.29 (s, 3H), 1.77 (s, 3H). LC-MS: 491.10[M + 1]+ |
| 47 | 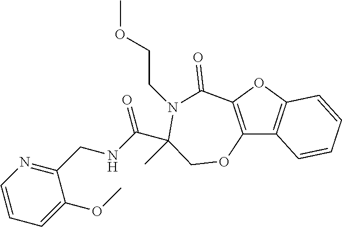 [SMILES: COCCN1C(=O)c2oc3ccccc3c2OCC1(C)C(=O)NCc1ncccc1OC] | 1H NMR (400 MHz, CDCl3) δ 7.95 (d, J = 4.2 Hz, 1H), 7.83 (s, 1H), 7.63 (d, J = 7.8 Hz, 1H), 7.49 − 7.37 (m, 2H), 7.25 − 7.18 (m, 1H), 7.09 − 6.94 (m, 2H), 4.83 (d, J = 11.8 Hz, 1H), 4.44 (qd, J = 17.1, 4.5 Hz, 2H), 4.27 (d, J = 12.1 Hz, 1H), 4.05 − 3.92 (m, 1H), 3.86 − 3.76 (m, 2H), 3.76 (s, 3H), 3.78 − 3.62 (m, 1H), 3.26 (s, 3H), 1.79 (s, 3H). LC-MS: 440.10[M + 1]+ |
| 48 | 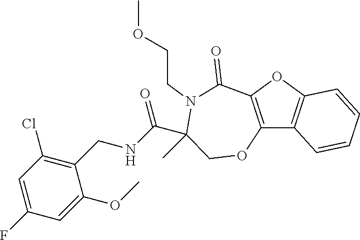 [SMILES: COCCN1C(=O)c2oc3ccccc3c2OCC1(C)C(=O)NCc1c(Cl)cc(F)cc1OC] | 1H NMR (400 MHz, DMSO) δ 7.86 (s, 1H), 7.65 − 7.53 (m, 2H), 7.53 − 7.43 (m, 1H), 7.30 (t, J = 7.5 Hz, 1H), 6.93 − 6.71 (m, 2H), 4.81 (d, J = 11.3 Hz, 1H), 4.40 − 4.17 (m, 3H), 3.74 (s, 3H), 3.67 − 3.55 (m, 2H), 3.48 (t, J = 5.9 Hz, 2H), 3.20 (s, 3H), 1.59 (s, 3H). LC-MS: 491.13[M + 1]+ |
| 49 | 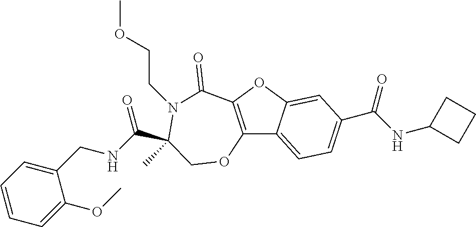 [SMILES: COCCN1C(=O)c2oc3cc(C(=O)NC4CCC4)ccc3c2OC[C@@]1(C)C(=O)NCc1ccccc1OC] | 1H NMR (400 MHz, CDCl3) δ 7.83 (s, 1H), 7.55 (d, J = 8.2 Hz, 1H), 7.54 − 7.49 (m, 1H), 7.49 − 7.40 (m, 1H), 6.93 − 6.78 (m, 2H), 6.57 (d, J = 8.0 Hz, 1H), 6.44 (t, J = 7.2 Hz, 1H), 6.26 (d, J = 7.5 Hz, 1H), 4.89 (d, J = 11.8 Hz, 1H), 4.70 − 4.56 (m, 1H), 4.35 (dd, J = 14.2, 7.0 Hz, 1H), 4.30 − 4.21 (m, 1H), 4.18 − 4.00 (m, 2H), 3.82 − 3.77 (m, 1H), 3.76 (s, 3H), 3.65 − 3.52 (m, 2H), 3.25 (s, 3H), 2.54 − 2.41 (m, 2H), 2.09 − 1.93 (m, 2H), 1.85 − 1.80 (m, 2H), 1.78 (s, 3H); LC-MS: 536.20[M + 1]+ |
| 50 | 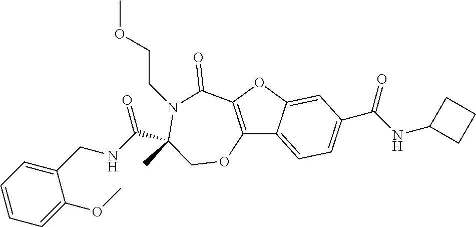 [SMILES: COCCN1C(=O)c2oc3cc(C(=O)NC4CCC4)ccc3c2OC[C@]1(C)C(=O)NCc1ccccc1OC] | LC-MS: 536.20[M + 1]+. 1H NMR (400 MHz, CDCl3) δ 7.82 (s, 1H), 7.59 − 7.49 (m, 2H), 7.45 (t, J = 5.2 Hz, 1H), 6.92-6.80 (m, 2H), 6.57 (d, J = 8.0 Hz, 1H), 6.44 (t, J = 7.2 Hz, 1H), 6.28 (d, J = 7.4 Hz, 1H), 4.89 (d, J = 11.7 Hz, 1H), 4.71 − 4.52 (m, 1H), 4.35 (dd, J = 14.2, 7.0 Hz, 1H), 4.30-4.20 (m, 1H), 4.18 − 4.01 (m, 2H), 3.84 − 3.77 (m, 1H), 3.76 (s, 3H), 3.68 − 3.53 (m, 2H), 3.25 (s, 3H), 2.56 − 2.38 (m, 2H), 2.15 − 1.94 (m, 2H), 1.88 − 1.79 (m, 2H), 1.78 (s, 3H). |
| 51 | 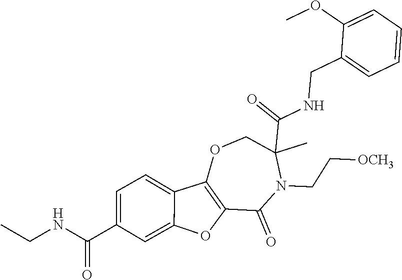 [SMILES: CCNC(=O)c1ccc2c3c(oc2c1)C(=O)N(CCOC)C(C)(C(=O)NCc1ccccc1OC)CO3] | LC-MS: 510.45 [M + H]+;1H NMR (400 MHz, CDCl3) δ (ppm): 7.78 (s, 1H), 7.50 (d, J = 19.0 Hz, 3H), 6.85 (d, J = 6.9 Hz, 2H), 6.56 (s, 1H), 6.41 (s, 1H), 6.23 (s, 1H), 4.94 − 4.80 (m, 1H), 4.32 (s, 2H), 4.06 (s, 2H), 3.74 (s, 4H), 3.53 (s, 4H), 3.23 (s, 3H), 1.76 (s, 3H), 1.28 (t, J = 7.2 Hz, 3H) |
| 52 | 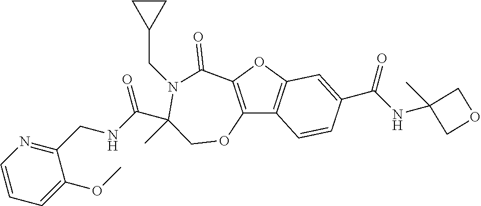 [SMILES: COc1cccnc1CNC(=O)C1(C)COc2c(oc3cc(C(=O)NC4(C)COC4)ccc23)C(=O)N1CC1CC1] | 1H NMR (400 MHz, CDCl3) δ 7.91 (d, J = 3.8 Hz, 1H), 7.85 (s, 1H), 7.75 (s, 1H), 7.67 − 7.57 (m, 2H), 7.08 − 6.98 (m, 2H), 6.65 (s, 1H), 4.89 (d, J = 6.5 Hz, 3H), 4.59 (d, J = 6.6 Hz, 2H), 4.43 (d, J = 4.5 Hz, 2H), 4.25 (d, J = 11.7 Hz, 1H), 3.83 − 3.55 (m, 5H), 1.86 (s, 3H), 1.79 (s, 3H), 1.21 − 1.10 (m, 1H), 0.62 − 0.41 (m, 4H). LC-MS: 549.2[M + 1]+ |
| 53 | 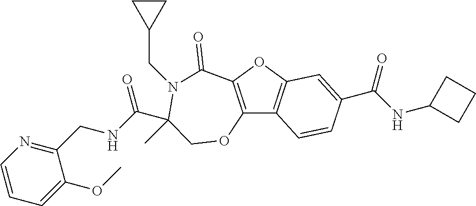 [SMILES: COc1cccnc1CNC(=O)C1(C)COc2c(oc3cc(C(=O)NC4CCC4)ccc23)C(=O)N1CC1CC1] | 1H NMR (400 MHz, CDCl3) δ 7.90 (d, J = 3.7 Hz, 1H), 7.84 (s, 1H), 7.75 (s, 1H), 7.64 (d, J = 8.2 Hz, 1H), 7.58 (d, J = 8.2 Hz, 1H), 7.08 − 6.98 (m, 2H), 6.25 (d, J = 7.4 Hz, 1H), 4.88 (d, J = 11.8 Hz, 1H), 4.60 (dd, J = 16.1, 8.2 Hz, 1H), 4.51 − 4.37 (m, 2H), 4.24 (d, J = 11.9 Hz, 1H), 3.80 − 3.59 (m, 5H), 2.52 − 2.39 (m, 2H), 2.04 − 1.93 (m, 2H), 1.86 (s, 3H), 1.84 − 1.80 (m, 2H), 1.21 − 1.08 (m, 1H), 0.60 − 0.43 (m, 4H). LC-MS: 533.2[M + 1]+ |
| 54 | 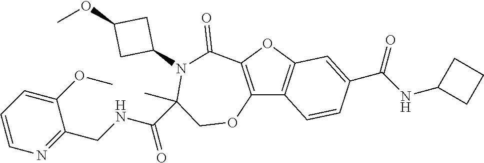 [SMILES: COc1cccnc1CNC(=O)C1(C)COc2c(oc3cc(C(=O)NC4CCC4)ccc23)C(=O)N1[C@H]1C[C@@H](OC)C1] | 1H NMR (400 MHz, CDCl3) δ 8.04 (s, 1H), 7.88 (s, 1H), 7.83 (s, 1H), 7.69 − 7.59 (m, 2H), 7.23 − 7.12 (m, 2H), 6.32 (d, J = 7.2 Hz, 1H), 4.68 − 4.54 (m, 3H), 4.51 − 4.39 (m, 2H), 3.87 (s, 3H), 3.70 − 3.52 (m, 2H), 3.21 (s, 3H), 3.03 − 2.83 (m, 2H), 2.68 − 2.56 (m, 1H), 2.53 − 2.35 (m, 3H), 2.08 − 1.95 (m, 2H), 1.83 − 1.76 (m, 2H), 1.67 (s, 3H). LC-MS: 563.2[M + 1]+ |
| 55 | 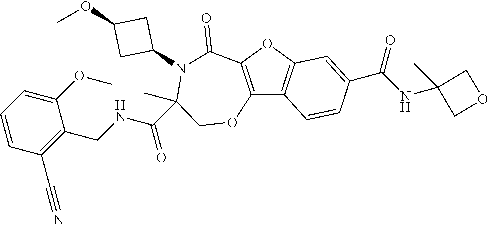 [SMILES: COc1cccc(C#N)c1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.02 (s, 1H), 8.46 (s, 1H), 8.05 (s, 1H), 7.85 − 7.75 (m, 2H), 7.49 (t, J = 8.0 Hz, 1H), 7.42 − 7.32 (m, 2H), 4.73 (d, J = 6.3 Hz, 2H), 4.53 (d, J = 12.6 Hz, 1H), 4.47 − 4.33 (m, 5H), 3.85 (s, 3H), 3.54 − 3.45 (m, 1H), 3.30 − 3.25 (m, 1H), 3.11 (s, 3H), 2.87 − 2.67 (m, 2H), 2.38 − 2.16 (m, 2H), 1.63 (s, 3H), 1.42 (s, 3H). LC-MS: 603.2[M + 1]+ |
| 56 | 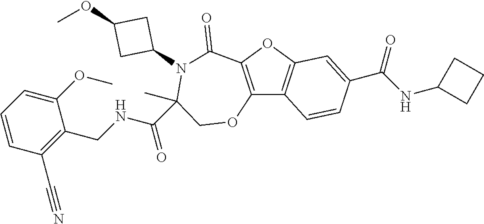 [SMILES: COc1cccc(C#N)c1CNC(=O)C1(C)COc2c(oc3cc(C(=O)NC4CCC4)ccc23)C(=O)N1[C@H]1C[C@@H](OC)C1] | 1H NMR (400 MHz, DMSO) δ 8.74 (d, J = 7.5 Hz, 1H), 8.46 (s, 1H), 8.04 (s, 1H), 7.85 − 7.72 (m, 2H), 7.49 (t, J = 8.0 Hz, 1H), 7.42 − 7.33 (m, 2H), 4.58 − 4.31 (m, 5H), 3.85 (s, 3H), 3.55 − 3.45 (m, 1H), 3.30 − 3.24 (m, 1H), 3.11 (s, 3H), 2.86 − 2.68 (m, 2H), 2.38 − 2.18 (m, 4H), 2.15 − 2.01 (m, 2H), 1.74 − 1.62 (m, 2H), 1.42 (s, 3H). LC- MS: 587.2[M + 1]+ |
| 57 | 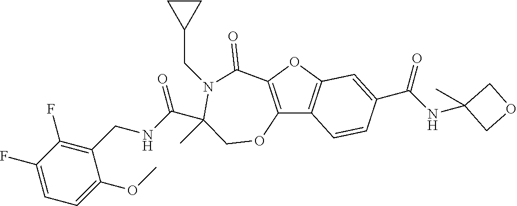 [SMILES: COc1ccc(F)c(F)c1CNC(=O)C1(C)COc2c(oc3cc(C(=O)NC4(C)COC4)ccc23)C(=O)N1CC1CC1] | 1H NMR (400 MHz, DMSO) δ 9.02 (s, 1H), 8.19 − 7.93 (m, 2H), 7.80 (dd, J = 8.3, 1.0 Hz, 1H), 7.69 (d, J = 8.3 Hz, 1H), 7.25 − 7.06 (m, 1H), 6.71 (d, J = 8.5 Hz, 1H), 4.84 − 4.69 (m, 3H), 4.44 − 4.19 (m, 5H), 3.80 − 3.62 (m, 4H), 2.97 (s, 1H), 1.76 − 1.51 (m, 6H), 1.08 − 0.95 (m, 1H), 0.48 − 0.38 (m, 2H), 0.34 − 0.17 (m, 2H). LC-MS: 584.2[M + 1]+ |
| 58 | 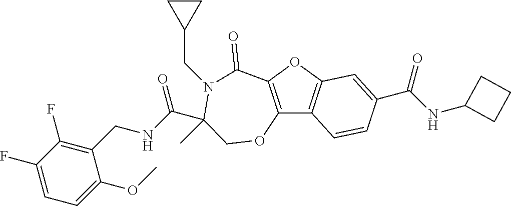 [SMILES: COc1ccc(F)c(F)c1CNC(=O)C1(C)COc2c(oc3cc(C(=O)NC4CCC4)ccc23)C(=O)N1CC1CC1] | 1H NMR (400 MHz, CDCl3) δ 7.84 (s, 1H), 7.57 − 7.46 (m, 2H), 6.99 (dd, J = 7.3, 4.1 Hz, 1H), 6.54 (dd, J = 18.6, 9.2 Hz, 1H), 6.32 (d, J = 7.7 Hz, 1H), 6.17 (d, J = 9.0 Hz, 1H), 4.91 (d, J = 11.9 Hz, 1H), 4.76 − 4.57 (m, 2H), 4.05 (d, J = 11.9 Hz, 1H), 3.97 (d, J = 12.6 Hz, 1H), 3.91 − 3.79 (m, 1H), 3.73 (s, 3H), 3.55 (dd, J = 14.9, 5.5 Hz, 1H), 2.54 − 2.42 (m, 2H), 2.09 − 1.96 (m, 2H), 1.88 − 1.78 (m, 5H), 1.13 − 1.00 (m, 1H), 0.69 − 0.58 (m, 2H), 0.54 − 0.43 (m, 2H). LC-MS: 568.2[M + 1]+ |
| 59 | 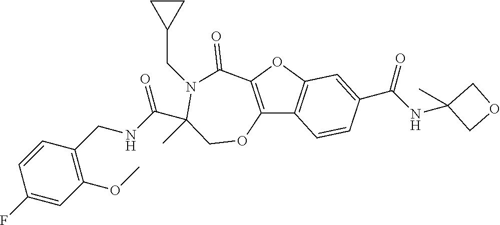 [SMILES: COc1cc(F)ccc1CNC(=O)C1(C)COc2c(oc3cc(C(=O)NC4(C)COC4)ccc23)C(=O)N1CC1CC1] | 1H NMR (400 MHz, CDCl3) δ 7.75 (s, 1H), 7.52 (dt, J = 15.6, 4.7 Hz, 2H), 7.06 (dd, J = 8.1, 3.9 Hz, 1H), 6.90 − 6.74 (m, 1H), 6.54 (s, 1H), 6.23 (dd, J = 11.0, 2.3 Hz, 1H), 6.08 − 5.93 (m, 1H), 4.92 (dd, J = 6.1, 2.2 Hz, 3H), 4.61 (d, J = 6.6 Hz, 2H), 4.39 (dd, J = 14.0, 8.4 Hz, 1H), 4.05 (d, J = 11.9 Hz, 1H), 3.96 (dd, J = 14.0, 3.9 Hz, 1H), 3.92 − 3.82 (m, 1H), 3.80 (s, 3H), 3.55 (dd, J = 15.0, 5.5 Hz, 1H), 1.87 (s, 3H), 1.82 (s, 3H), 1.16 − 0.99 (m, 1H), 0.77 − 0.43 (m, 4H). LC-MS: 566.2[M + 1]+ |
| 60 | 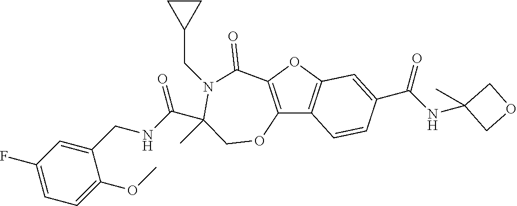 [SMILES: COc1ccc(F)cc1CNC(=O)C1(C)COc2c(oc3cc(C(=O)NC4(C)COC4)ccc23)C(=O)N1CC1CC1] | 1H NMR (400 MHz, CDCl3) δ 7.73 (s, 1H), 7.56 − 7.45 (m, 2H), 7.21 (s, 1H), 6.67 (d, J = 11.8 Hz, 2H), 6.47-6.45 (m, 2H), 4.92 (dd, J = 6.7, 2.9 Hz, 3H), 4.62 (d, J = 6.5 Hz, 2H), 4.35 (dd, J = 14.3, 7.6 Hz, 1H), 4.17 − 4.03 (m, 2H), 3.81-3.79 (m, 1H), 3.75 (s, 3H), 3.60 (dd, J = 14.9, 5.6 Hz, 1H), 1.86 (s, 3H), 1.82 (s, 3H), 1.13 − 0.97 (m, 1H), 0.64 − 0.47 (m, 4H); LC-MS: 566.2[M + 1]+ |
| 61 | 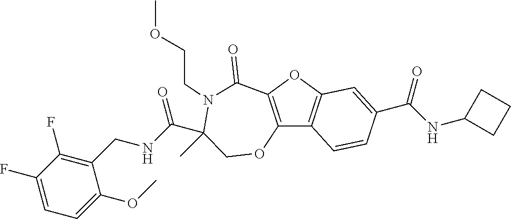 [SMILES: COCCN1C(=O)c2oc3cc(C(=O)NC4CCC4)ccc3c2OCC1(C)C(=O)NCc1c(OC)ccc(F)c1F] | 1H NMR (400 MHz, CDCl3) δ 7.83 (s, 1H), 7.57 (d, J = 8.1 Hz, 1H), 7.52 − 7.48 (m, 2H), 6.69 − 6.46 (m, 1H), 6.26 (d, J = 7.6 Hz, 1H), 6.21 (d, J = 9.3 Hz, 1H), 4.89 (d, J = 11.8 Hz, 1H), 4.71 − 4.60 (m, 2H), 4.37 − 4.34 (m, 1H), 4.08 (d, J = 11.7 Hz, 1H), 3.95 (d, J = 13.4 Hz, 1H), 3.83 − 3.76 (m, 1H), 3.71 (s, 3H), 3.62 − 3.44 (m, 2H), 3.31 (s, 3H), 2.64 − 2.37 (m, 2H), 2.09 − 1.92 (m, 2H), 1.83 (dd, J = 9.6, 5.2 Hz, 2H), 1.79 (d, J = 6.5 Hz, 3H). LC-MS: 572.2[M + 1]+ |
| 62 | 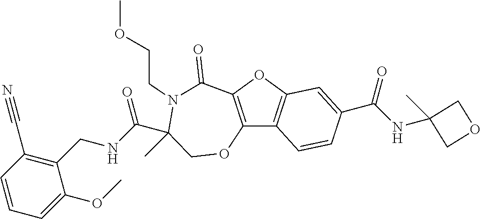 [SMILES: COCCN1C(=O)c2oc3cc(C(=O)NC4(C)COC4)ccc3c2OCC1(C)C(=O)NCc1c(C#N)cccc1OC] | 1H NMR (400 MHz, CDCl3) δ 7.76 (s, 1H), 7.69 (t, J = 5.1 Hz, 1H), 7.51 − 7.50 (m, 2H), 6.90 (t, J = 7.8 Hz, 1H), 6.83 − 6.80 (m, 2H), 6.75 (d, J = 8.2 Hz, 1H), 4.94 − 4.88 (m, 3H), 4.74 (dd, J = 14.1, 7.0 Hz, 1H), 4.61 (dd, J = 6.4, 1.7 Hz, 2H), 4.32 − 4.31 (m, 1H), 4.15 (dd, J = 14.1, 3.9 Hz, 1H), 4.07 (d, J = 11.9 Hz, 1H), 3.86 − 3.80 (m, 1H), 3.76 (s, 3H), 3.69 − 3.51 (m, 2H), 3.33 (s, 3H), 1.82 (s, 3H), 1.79 (s, 3H). LC-MS: 577.2[M + 1]+ |
| 63 | 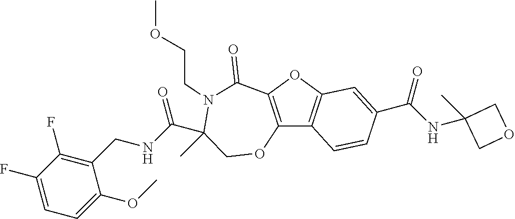 [SMILES: COCCN1C(=O)c2oc3cc(C(=O)NC4(C)COC4)ccc3c2OCC1(C)C(=O)NCc1c(OC)ccc(F)c1F] | 1H NMR (400 MHz, CDCl3) δ 7.82 (s, 1H), 7.58 (d, J = 8.0 Hz, 1H), 7.51 (dd, J = 8.2, 1.3 Hz, 1H), 7.49 − 7.44 (m, 1H), 6.68 − 6.54 (m, 2H), 6.25 (d, J = 8.0 Hz, 1H), 4.93 − 4.88 (m, 3H), 4.69 − 4.58 (m, 3H), 4.34 − 4.31 (m, 1H), 4.10 (d, J = 11.8 Hz, 1H), 3.97 (d, J = 13.1 Hz, 1H), 3.83 − 3.77 (m, 1H), 3.73 (s, 3H), 3.62 −3.52 (m, 2H), 3.31 (s, 3H), 1.82 (s, 3H), 1.78 (s, 3H). LC-MS: 588.2[M + 1]+ |
| 64 | 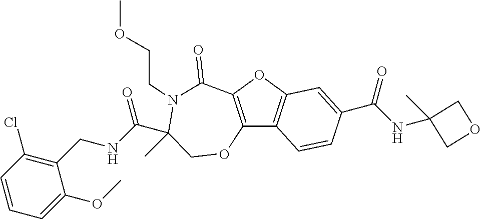 [SMILES: COCCN1C(=O)c2oc3cc(C(=O)NC4(C)COC4)ccc3c2OCC1(C)C(=O)NCc1c(Cl)cccc1OC] | 1H NMR (400 MHz, CDCl3) δ 7.83 (s, 1H), 7.60 (d, J = 8.2 Hz, 1H), 7.54 (dd, J = 8.2, 1.3 Hz, 1H), 7.30 (t, J = 8.9 Hz, 1H), 6.86 (t, J = 8.2 Hz, 1H), 6.64 (d, J = 8.0 Hz, 1H), 6.58 (s, 1H), 6.53 (d, J = 8.3 Hz, 1H), 4.92 − 4.87 (m, 3H), 4.70 (dd, J = 13.8, 6.7 Hz, 1H), 4.61 (d, J = 6.7 Hz, 2H), 4.33 − 4.04 (m, 3H), 3.84 − 3.76 (m, 1H), 3.73 (s, 3H), 3.61 − 3.50 (m, 2H), 3.18 (s, 3H), 1.81 (s, 3H), 1.78 (s, 3H); LC-MS: 586.2[M + 1]+ |
| 65 | 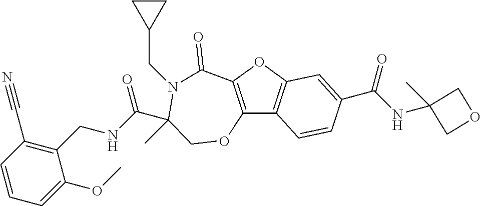 [SMILES: COc1cccc(C#N)c1CNC(=O)C1(C)COc2c(oc3cc(C(=O)NC4(C)COC4)ccc23)C(=O)N1CC1CC1] | 1H NMR (400 MHz, CDCl3) δ 7.80 (s, 1H), 7.53 − 7.44 (m, 2H), 7.08 (s, 1H), 6.90 − 6.82 (m, 1H), 6.80 (d, J = 6.8 Hz, 1H), 6.71 (d, J = 7.6 Hz, 1H), 6.58 (s, 1H), 4.94 − 4.91 (m, 3H), 4.79 (dd, J = 14.2, 7.6 Hz, 1H), 4.62 (dd, J = 6.4, 1.6 Hz, 2H), 4.17 (dd, J = 14.1, 4.1 Hz, 1H), 4.03 (d, J = 11.9 Hz, 1H), 3.79 − 3.78 (m, 1H), 3.77 (s, 3H), 3.59 (dd, J = 15.0, 5.6 Hz, 1H), 1.88 (s, 3H), 1.82 (s, 3H), 1.10 − 1.07 (m, 1H), 0.68 − 0.59 (m, 2H), 0.52 − 0.49 (m, 2H). LC-MS: 573.2[M + 1]+ |
| 66 | 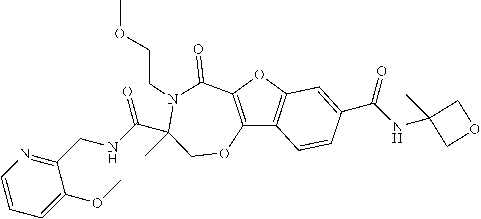 [SMILES: COCCN1C(=O)c2oc3cc(C(=O)NC4(C)COC4)ccc3c2OCC1(C)C(=O)NCc1ncccc1OC] | 1H NMR (400 MHz, CDCl3) δ 7.94 − 7.85 (m, 3H), 7.65 (d, J = 8.2 Hz, 1H), 7.60 (d, J = 8.2 Hz, 1H), 7.09 − 6.98 (m, 2H), 6.70 (s, 1H), 4.90 − 4.85 (m, 3H), 4.59 (d, J = 6.5 Hz, 2H), 4.48 − 4.20 (m, 3H), 4.03 − 3.95 (m, 1H), 3.90 − 3.78 (m, 2H), 3.76 (s, 3H), 3.71 − 3.64 (m, 1H), 3.26 (s, 3H), 1.81 (s, 3H), 1.79 (s, 3H). LC-MS: 553.2[M + 1]+ |
| 67 | 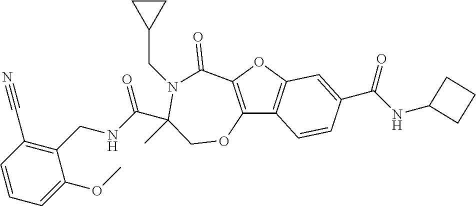 [SMILES: COc1cccc(C#N)c1CNC(=O)C1(C)COc2c(oc3cc(C(=O)NC4CCC4)ccc23)C(=O)N1CC1CC1] | 1H NMR (400 MHz, CDCl3) δ 7.83 (s, 1H), 7.53 − 7.41 (m, 2H), 7.06 − 7.02 (m, 1H), 6.86 − 6.74 (m, 2H), 6.67 (d, J = 8.0 Hz, 1H), 6.27 (d, J = 7.3 Hz, 1H), 4.92 (d, J = 12.0 Hz, 1H), 4.82 (dd, J = 14.1, 7.9 Hz, 1H), 4.63 (dd, J = 16.1, 8.0 Hz, 1H), 4.11 (dd, J = 14.1, 4.3 Hz, 1H), 4.02 (d, J = 11.9 Hz, 1H), 3.85 − 3.80 (m, 1H), 3.77 (s, 3H), 3.57 (dd, J = 14.8, 5.5 Hz, 1H), 2.52 − 2.45 (m, 2H), 2.06 − 1.96 (m, 2H), 1.88 (s, 3H), 1.86 − 1.77 (m, 2H), 1.13 − 1.09 (m, 1H), 0.74 − 0.59 (m, 2H), 0.54 − 0.50 (m, 2H). LC-MS: 557.2[M + 1]+ |
| 68 | 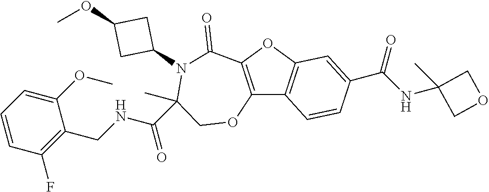 [SMILES: COc1cccc(F)c1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.02 (s, 1H), 8.31 (s, 1H), 8.05 (s, 1H), 7.85-7.74 (m, 2H), 7.32 (dd, J = 15.3, 8.2 Hz, 1H), 6.87 (d, J = 8.4 Hz, 1H), 6.80 (t, J = 8.8 Hz, 1H), 4.73 (d, J = 6.2 Hz, 2H), 4.56 (d, J = 12.7 Hz, 1H), 4.43 − 4.22 (m, 5H), 3.82 (s, 3H), 3.55 − 3.41 (m, 1H), 3.30-3.21 (m, 1H), 3.11 (s, 3H), 2.85-2.65 (m, 2H), 2.36-2.13 (m, 2H), 1.63 (s, 3H), 1.44 (s, 3H). LC-MS: 596.20[M + 1]+ |
| 69 | 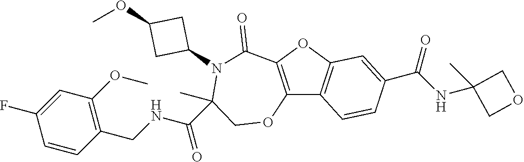 [SMILES: COc1cc(F)ccc1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.03 (s, 1H), 8.49 (s, 1H), 8.06 (s, 1H), 7.86-7.76 (m, 2H), 7.20 (s, 1H), 6.89 (d, J = 11.3 Hz, 1H), 6.68 (s, 1H), 4.74 (d, J = 6.3 Hz, 2H), 4.62 (d, J = 12.3 Hz, 1H), 4.47-4.35 (m, 3H), 4.34-4.13 (m, 2H), 3.80 (s, 3H), 3.57 − 3.41 (m, 1H), 3.31-3.28 (m, 1H), 3.11 (s, 3H), 2.93 − 2.62 (m, 2H), 2.39-2.14 (m, 2H), 1.63 (s, 3H), 1.45 (s, 3H). LC-MS: 596.20[M + 1]+ |
| 70 | 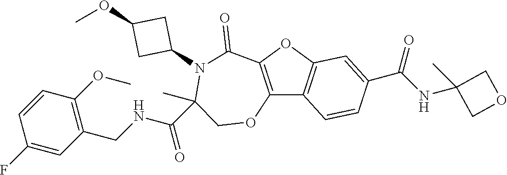 [SMILES: COc1ccc(F)cc1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.03 (s, 1H), 8.56 (s, 1H), 8.06 (s, 1H), 7.94 − 7.71 (m, 2H), 7.17 − 6.81 (m, 3H), 4.74 (d, J = 6.3 Hz, 2H), 4.64 (d, J = 12.3 Hz, 1H), 4.45 (d, J = 12.7 Hz, 1H), 4.40 (d, J = 6.4 Hz, 2H), 4.38-4.29 (m, 1H), 4.27-4.15 (m, 1H), 3.78 (s, 3H), 3.56-3.41 (m, 1H), 3.31 − 3.26 (m, 1H), 3.11 (s, 3H), 2.92 − 2.66 (m, 2H), 2.39- 2.22 (m, 2H), 1.63 (s, 3H), 1.47 (s, 3H). LC- MS: 596.20[M + 1]+ |
| 71 | 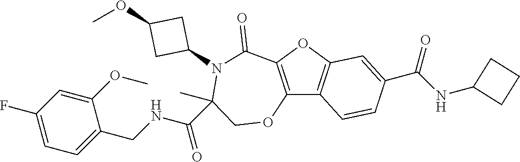 [SMILES: COc1cc(F)ccc1CNC(=O)C1(C)COc2c(oc3cc(C(=O)NC4CCC4)ccc23)C(=O)N1[C@H]1C[C@@H](OC)C1] | 1H NMR (400 MHz, DMSO) δ 8.75 (d, J = 7.5 Hz, 1H), 8.55-8.45 (m, 1H), 8.06 (s, 1H), 7.83 (dd, J = 8.3, 1.2 Hz, 1H), 7.77 (d, J = 8.2 Hz, 1H), 7.25-7.10 (m, 1H), 6.89 (d, J = 10.7 Hz, 1H), 6.75-6.60 (m, 1H), 4.61 (d, J = 12.4 Hz, 1H), 4.52 − 4.37 (m, 2H), 4.35-4.15 (m, 2H), 3.80 (s, 3H), 3.45 (dd, J = 14.0, 7.1 Hz, 1H), 3.31 − 3.25 (m, 1H), 3.11 (s, 3H), 2.87-2.65 (m, 2H), 2.38 − 2.17 (m, 4H), 2.15-2.00 (m, 2H), 1.78 − 1.59 (m, 2H), 1.44 (s, 3H). LC-MS: 580.20[M + 1]+ |
| 72 | 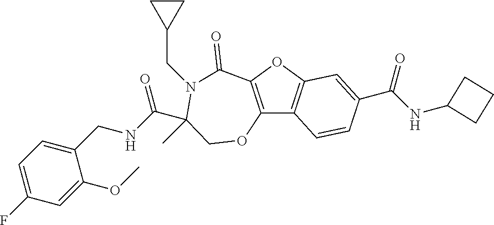 [SMILES: COc1cc(F)ccc1CNC(=O)C1(C)COc2c(oc3cc(C(=O)NC4CCC4)ccc23)C(=O)N1CC1CC1] | 1H NMR (400 MHz, DMSO) δ 8.74 (d, J = 7.5 Hz, 1H), 8.26 (s, 1H), 8.05 (s, 1H), 7.83 (dd, J = 8.3, 1.2 Hz, 1H), 7.74 (d, J = 8.3 Hz, 1H), 6.96 − 6.66 (m, 2H), 6.22 (s, 1H), 4.91 (d, J = 12.0 Hz, 1H), 4.58-4.30 (m, 2H), 4.21-4.00 (m, 2H), 3.73 (s, 3H), 3.73- 3.69 (m, 1H), 3.23 − 2.98 (m, 1H), 2.34 − 2.18 (m, 2H), 2.18 − 1.97 (m, 2H), 1.87 − 1.52 (m, 5H), 1.15-0.93 (m, 1H), 0.52 − 0.38 (m, 2H), 0.37-0.15 (m, 2H). LC-MS: 550.20[M + 1]+ |
| 73 | 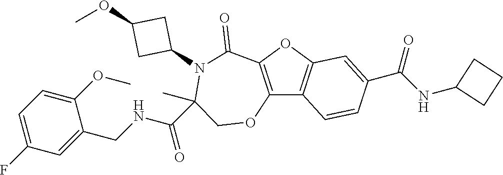 [SMILES: COc1ccc(F)cc1CNC(=O)C1(C)COc2c(oc3cc(C(=O)NC4CCC4)ccc23)C(=O)N1[C@H]1C[C@@H](OC)C1] | 1H NMR (400 MHz, DMSO) δ 8.75 (d, J = 7.5 Hz, 1H), 8.57 (s, 1H), 8.06 (s, 1H), 7.83 (dd, J = 8.3, 1.2 Hz, 1H), 7.75 (d, J = 8.3 Hz, 1H), 7.15 − 6.90 (m, 3H), 4.64 (d, J = 12.0 Hz, 1H), 4.52 − 4.39 (m, 2H), 4.34 (dd, J = 15.4, 5.8 Hz, 1H), 4.21 (dd, J = 15.2, 5.0 Hz, 1H), 3.78 (s, 3H), 3.55-3.44 (m, 1H), 3.30-3.24 (m, 1H), 3.11 (s, 3H), 2.90- 2.65 (m, 2H), 2.43 − 2.17 (m, 5H), 2.17 − 1.99 (m, 2H), 1.78 − 1.60 (m, 2H), 1.46 (s, 3H). LC-MS: 580.20[M + 1]+ |
| 74 | 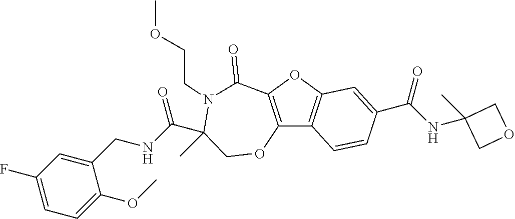 [SMILES: COCCN1C(=O)c2oc3cc(C(=O)NC4(C)COC4)ccc3c2OCC1(C)C(=O)NCc1cc(F)ccc1OC] | 1H NMR (400 MHz, CDCl3) δ 7.79 (s, 1H), 7.62-7.54 (m, 2H), 7.51 (dd, J = 8.2, 1.3 Hz, 1H), 6.66 − 6.43 (m, 4H), 4.97-4.82 (m, 3H), 4.61 (d, J = 6.7 Hz, 2H), 4.32 (dd, J = 14.4, 7.3 Hz, 2H), 4.17 − 3.97 (m, 2H), 3.90 − 3.79 (m, 1H), 3.76 (s, 3H), 3.66-3.51 (m, 2H), 3.32 (s, 3H), 1.81 (s, 3H), 1.80 (s, 3H). LC-MS: 570.20[M + 1]+ |
| 75 | 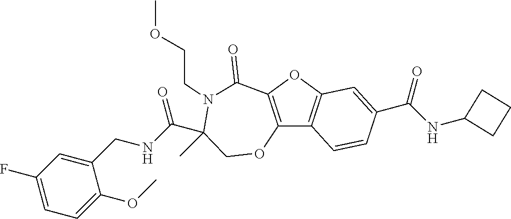 [SMILES: COCCN1C(=O)c2oc3cc(C(=O)NC4CCC4)ccc3c2OCC1(C)C(=O)NCc1cc(F)ccc1OC] | 1H NMR (400 MHz, CDCl3) δ 7.82 (s, 1H), 7.59 − 7.52 (m, 2H), 7.53 − 7.45 (m, 1H), 6.62 (d, J = 8.1 Hz, 1H), 6.52 − 6.42 (m, 2H), 6.28 (d, J = 7.4 Hz, 1H), 4.90 (d, J = 11.9 Hz, 1H), 4.74 − 4.55 (m, 1H), 4.44 − 4.26 (m, 2H), 4.18 − 3.94 (m, 2H), 3.86 − 3.78 (m, 1H), 3.75 (s, 3H), 3.73 − 3.52 (m, 2H), 3.32 (s, 3H), 2.55 − 2.36 (m, 2H), 2.14 − 1.92 (m, 2H), 1.88 − 1.80 (m, 2H), 1.79 (s, 3H). LC-MS: 554.20[M + 1]+ |
| 76 | 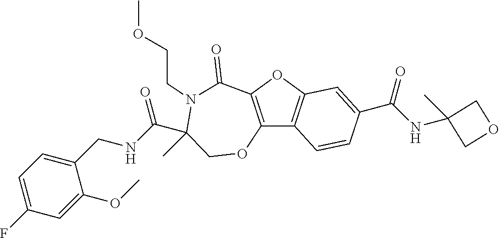 [SMILES: COCCN1C(=O)c2oc3cc(C(=O)NC4(C)COC4)ccc3c2OCC1(C)C(=O)NCc1ccc(F)cc1OC] | 1H NMR (400 MHz, CDCl3) δ 7.73 (s, 1H), 7.62 − 7.47 (m, 4H), 6.86 − 6.75 (m, 1H), 6.58 (s, 1H), 6.27 (dd, J = 11.0, 2.2 Hz, 1H), 5.99 (t, J = 7.4 Hz, 1H), 4.95-4.90 (m, 2H), 4.61 (d, J = 6.7 Hz, 2H), 4.42-4.30 (m, 2H), 4.08 (d, J = 11.8 Hz, 1H), 3.99 − 3.89 (m, 1H), 3.85 − 3.73 (m, 5H), 3.63 − 3.53 (m, 2H), 3.36 (s, 3H), 1.81 (s, 3H), 1.79 (s, 3H). LC-MS: 570.20[M + 1]+ |
| 77 | 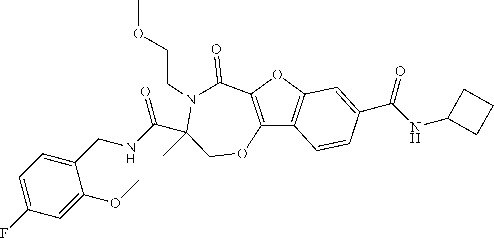 [SMILES: COCCN1C(=O)c2oc3cc(C(=O)NC4CCC4)ccc3c2OCC1(C)C(=O)NCc1ccc(F)cc1OC] | 1H NMR (400 MHz, CDCl3) δ 7.73 (s, 1H), 7.61 − 7.43 (m, 3H), 6.81 (t, J = 7.5 Hz, 1H), 6.35 (d, J = 7.0 Hz, 1H), 6.26 (dd, J = 11.0, 2.0 Hz, 1H), 6.02 (t, J = 7.5 Hz, 1H), 4.88 (d, J = 11.8 Hz, 1H), 4.69 − 4.54 (m, 1H), 4.36 (dd, J = 14.1, 7.8 Hz, 2H), 4.07 (d, J = 11.9 Hz, 1H), 3.94 (dd, J = 13.8, 3.9 Hz, 1H), 3.85 − 3.72 (m, 4H), 3.66 − 3.51 (m, 2H), 3.35 (m, 3H), 2.56 − 2.39 (m, 2H), 2.15 − 1.94 (m, 2H), 1.87 − 1.80 (m, 2H), 1.79 (s, 3H). LC-MS: 554.20[M + 1]+ |
| 78 | 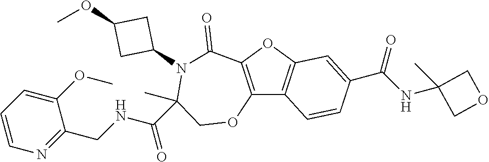 [SMILES: COc1cccnc1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.03 (s, 1H), 8.48 (s, 1H), 8.11 − 8.02 (m, 2H), 7.87 − 7.74 (m, 2H), 7.41 (d, J = 8.3 Hz, 1H), 7.29 (dd, J = 8.3, 4.7 Hz, 1H), 4.74 (d, J = 6.3 Hz, 2H), 4.60 (d, J = 12.7 Hz, 1H), 4.51 − 4.32 (m, 5H), 3.84 (s, 3H), 3.56 − 3.42 (m, 2H), 3.11 (s, 3H), 2.90 − 2.72 (m, 2H), 2.45 − 2.40 (m, 1H), 2.32 − 2.20 (m, 1H), 1.63 (s, 3H), 1.44 (s, 3H); LC-MS: 579.20[M + 1]+ |
| 79 | 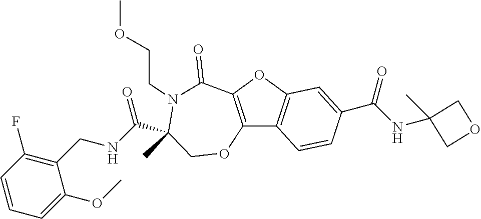 [SMILES: COCCN1C(=O)c2oc3cc(C(=O)NC4(C)COC4)ccc3c2OC[C@]1(C)C(=O)NCc1c(F)cccc1OC] | 1H NMR (400 MHz, CDCl3) δ 7.83 (s, 1H), 7.61 (d, J = 8.2 Hz, 1H), 7.57 − 7.49 (m, 1H), 7.35 (t, J = 5.6 Hz, 1H), 6.93 − 6.81 (m, 1H), 6.48 (s, 1H), 6.39 (d, J = 8.5 Hz, 1H), 6.34 (t, J = 8.4 Hz, 1H), 4.96 − 4.83 (m, 3H), 4.65 − 4.52 (m, 3H), 4.36 − 4.19 (m, 1H), 4.16 − 4.02 (m, 2H), 3.85 − 3.75 (m, 1H), 3.73 (s, 3H), 3.63 − 3.49 (m, 2H), 3.22 (s, 3H), 1.81 (s, 3H), 1.77 (s, 3H). LC-MS: 570.20[M + 1]+ |
| 80 | 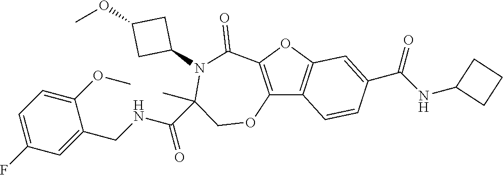 [SMILES: COc1ccc(F)cc1CNC(=O)C1(C)COc2c(oc3cc(C(=O)NC4CCC4)ccc23)C(=O)N1[C@H]1C[C@H](OC)C1] | 1H NMR (400 MHz, DMSO) δ 8.75 (d, J = 7.3 Hz, 1H), 8.59 − 8.41 (m, 1H), 8.05 (s, 1H), 7.83 (d, J = 8.3 Hz, 1H), 7.76 (d, J = 8.2 Hz, 1H), 7.15 − 6.87 (m, 3H), 4.70 − 4.59 (m, 1H), 4.51 − 4.38 (m, 2H), 4.27 (d, J = 5.4 Hz, 2H), 4.05 − 3.96 (m, 1H), 3.85 (s, 1H), 3.78 (s, 3H), 3.13 (d, J = 30.2 Hz, 3H), 3.03 − 2.84 (m, 2H), 2.27 − 2.17 (m, 2H), 2.14 − 2.03 (m, 3H), 2.00 − 1.87 (m, 1H), 1.75 − 1.61 (m, 2H), 1.41 (s, 3H). LC-MS: 580.20[M + 1]+ |
| 81 | 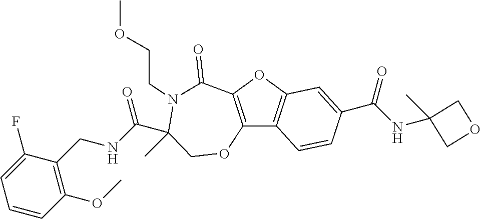 [SMILES: COCCN1C(=O)c2oc3cc(C(=O)NC4(C)COC4)ccc3c2OCC1(C)C(=O)NCc1c(F)cccc1OC] | 1H NMR (400 MHz, DMSO) δ 9.01 (s, 1H), 8.09 − 7.93 (m, 2H), 7.79 (dd, J = 8.3, 1.2 Hz, 1H), 7.68 (d, J = 8.1 Hz, 1H), 7.25 − 7.08 (m, 1H), 6.75 (d, J = 8.3 Hz, 1H), 6.67 − 6.51 (m, 1H), 4.82 (d, J = 11.2 Hz, 1H), 4.74 (d, J = 6.4 Hz, 2H), 4.40 (d, J = 6.4 Hz, 2H), 4.36 − 4.11 (m, 3H), 3.72 (s, 3H), 3.68 − 3.58 (m, 2H), 3.54 − 3.42 (m, 2H), 3.19 (s, 3H), 1.64 (s, 3H), 1.59 (s, 3H). LC-MS: 570.20[M + 1]+ |
| 82 | 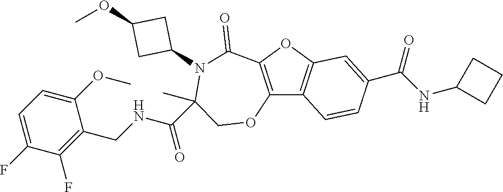 | 1H NMR (400 MHz, DMSO) δ 8.74 (d, J = 7.5 Hz, 1H), 8.39 (s, 1H), 8.05 (s, 1H), 7.82 (dd, J = 8.3, 1.3 Hz, 1H), 7.75 (d, J = 8.3 Hz, 1H), 7.35 (q, J = 9.6 Hz, 1H), 6.84 (d, J = 8.0 Hz, 1H), 4.52 (d, J = 12.6 Hz, 1H), 4.49 − 4.23 (m, 4H), 3.80 (s, 3H), 3.52 − 3.41 (m, 1H), 3.30 − 3.22 (m, 1H), 3.10 (s, 3H), 2.87 − 2.64 (m, 2H), 2.32 − 2.17 (m, 4H), 2.17 − 2.01 (m, 2H), 1.78 − 1.62 (m, 2H), 1.40 (s, 3H). LC-MS: 298.20[M + 1]+ |
| 83 | 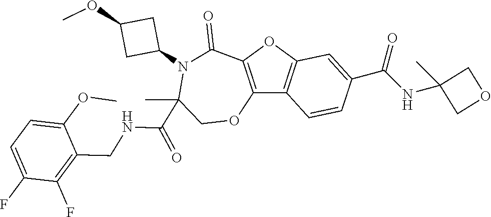 [SMILES: COc1ccc(F)c(F)c1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.02 (s, 1H), 8.39 (s, 1H), 8.05 (s, 1H), 7.82 (dd, J = 8.3, 1.2 Hz, 1H), 7.77 (d, J = 8.2 Hz, 1H), 7.35 (dd, J = 19.0, 9.4 Hz, 1H), 6.84 (d, J = 7.4 Hz, 1H), 4.73 (d, J = 6.2 Hz, 2H), 4.52 (d, J = 12.4 Hz, 1H), 4.45 − 4.24 (m, 5H), 3.80 (s, 3H), 3.53 − 3.42 (m, 1H), 3.28 − 3.19 (m, 1H), 3.10 (s, J = 7.6 Hz, 3H), 2.87 − 2.64 (m, 2H), 2.34 − 2.15 (m, 2H), 1.63 (s, 3H), 1.41 (s, 3H); LC-MS: 614.20[M + 1]+ |
| 84 | 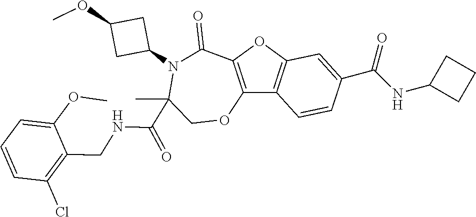 [SMILES: COc1cccc(Cl)c1CNC(=O)C1(C)COc2c(oc3cc(C(=O)NC4CCC4)ccc23)C(=O)N1[C@H]1C[C@@H](OC)C1] | 1H NMR (400 MHz, DMSO) δ 8.74 (d, J = 7.5 Hz, 1H), 8.22 (s, 1H), 8.04 (s, 1H), 7.86 − 7.72 (m, 2H), 7.31 (t, J = 8.2 Hz, 1H), 7.03 (dd, J = 10.4, 8.4 Hz, 2H), 4.57 (d, J = 12.7 Hz, 1H), 4.50 − 4.37 (m, 3H), 4.33 (d, J = 12.7 Hz, 1H), 3.81 (s, 3H), 3.57 − 3.48 (m, 1H), 3.30 − 3.23 (m, 1H), 3.12 (s, 3H), 2.89 − 2.71 (m, 2H), 2.39 − 2.19 (m, 4H), 2.15 − 2.02 (m, 2H), 1.74 − 1.64 (m, 2H), 1.41 (s, 3H); LC-MS: 597.2[M + 1]+ |
| 85 | 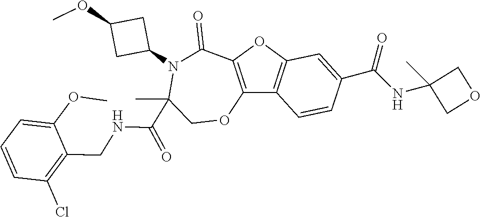 [SMILES: COc1cccc(Cl)c1CNC(=O)C1(C)COc2c(oc3cc(C(=O)NC4(C)COC4)ccc23)C(=O)N1[C@H]1C[C@@H](OC)C1] | 1H NMR (400 MHz, DMSO) δ 9.02 (s, 1H), 8.22 (s, 1H), 8.05 (s, 1H), 7.85 − 7.72 (m, 2H), 7.31 (t, J = 8.2 Hz, 1H), 7.04 (dd, J = 10.0, 8.5 Hz, 2H), 4.73 (d, J = 6.2 Hz, 2H), 4.58 (d, J = 12.6 Hz, 1H), 4.48 − 4.30 (m, 5H), 3.82 (s, 3H), 3.56 − 3.47 (m, 1H), 3.29 − 3.24 (m, 1H), 3.12 (s, 3H), 2.91 − 2.73 (m, 2H), 2.40 − 2.19 (m, 2H), 1.63 (s, 3H), 1.41 (s, 3H). LC-MS: 612.2.[M + 1]+ |
| 86 | 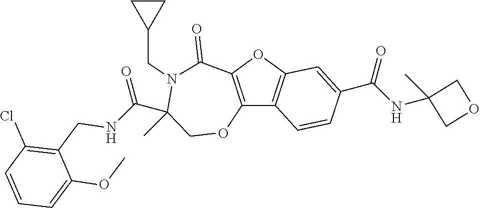 [SMILES: COc1cccc(Cl)c1CNC(=O)C1(C)COc2c(oc3cc(C(=O)NC4(C)COC4)ccc23)C(=O)N1CC1CC1] | 1H NMR (400 MHz, CDCl3) δ 7.83 (s, 1H), 7.61 − 7.50 (m, 2H), 6.81 (t, J = 8.1 Hz, 2H), 6.59 (d, J = 9.1 Hz, 2H), 6.49 (d, J = 8.3 Hz, 1H), 4.96 − 4.86 (m, 3H), 4.76 (dd, J = 13.9, 7.1 Hz, 1H), 4.61 (d, J = 6.5 Hz, 2H), 4.21 (dd, J = 13.8, 4.2 Hz, 1H), 4.07 (d, J = 11.9 Hz, 1H), 3.88 − 3.76 (m, 1H), 3.72 (s, 3H), 3.53 (dd, J = 14.9, 5.4 Hz, 1H), 1.86 (s, 3H), 1.81 (s, 3H), 1.10 − 0.98 (m, 1H), 0.62 − 0.52 (m, 2H), 0.51 − 0.42 (m, 1H), 0.42 − 0.34 (m, 1H). LC-MS: 582.2[M + 1]+ |
| 87 | 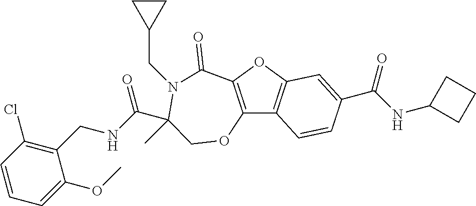 [SMILES: COc1cccc(Cl)c1CNC(=O)C1(C)COc2c(oc3cc(C(=O)NC4CCC4)ccc23)C(=O)N1CC1CC1] | 1H NMR (400 MHz, CDCl3) δ 7.82 (s, 1H), 7.53 (dt, J = 8.2, 4.7 Hz, 2H), 6.86 − 6.73 (m, 2H), 6.57 (d, J = 8.1 Hz, 1H), 6.47 (d, J = 8.3 Hz, 1H), 6.29 (d, J = 7.6 Hz, 1H), 4.91 (d, J = 11.8 Hz, 1H), 4.80 (dd, J = 13.8, 7.3 Hz, 1H), 4.68 − 4.55 (m, 1H), 4.17 (dd, J = 13.9, 4.0 Hz, 1H), 4.06 (d, J = 11.9 Hz, 1H), 3.91 − 3.78 (m, 1H), 3.71 (s, 3H), 3.52 (dd, J = 14.7, 5.3 Hz, 1H), 2.54 − 2.42 (m, 2H), 2.07 − 1.95 (m, 2H), 1.89 − 1.78 (m, 5H), 1.10 − 1.00 (m, 1H), 0.64 − 0.52 (m, 2H), 0.50 − 0.36 (m, 2H). LC-MS: 566.2[M + 1]+ |
| 88 | 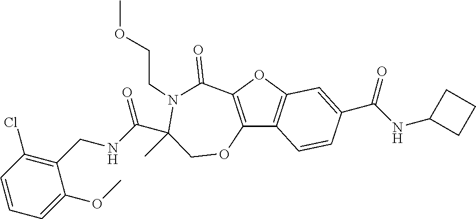 [SMILES: COCCN1C(=O)c2oc3cc(C(=O)NC4CCC4)ccc3c2OCC1(C)C(=O)NCc1c(Cl)cccc1OC] | 1H NMR (400 MHz, CDCl3) δ 7.82 (s, 1H), 7.58 (d, J = 8.2 Hz, 1H), 7.52 (d, J = 4 Hz, 1H), 7.31 (s, 1H), 6.84 (t, J = 8.1 Hz, 1H), 6.62 (d, J = 8.0 Hz, 1H), 6.51 (d, J = 8.3 Hz, 1H), 6.27 (d, J = 7.2 Hz, 1H), 4.88 (d, J = 11.7 Hz, 1H), 4.74 (dd, J = 13.8, 6.9 Hz, 1H), 4.62 (dd, J = 16.2, 8.3 Hz, 1H), 4.36 − 4.01 (m, 3H), 3.78 (dt, J = 11.3, 3.9 Hz, 1H), 3.74 (d, J = 8.4 Hz, 3H), 3.55 (dq, J = 9.7, 3.8 Hz, 2H), 3.20 (s, 3H), 2.59 − 2.36 (m, 2H), 2.12 − 1.89 (m, 2H), 1.82-1.80 (m, 2H), 1.78 (s, 3H). LC-MS: 570.2[M + 1]+ |
| 89 | 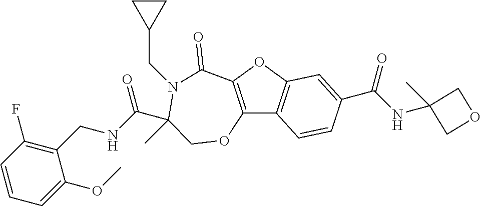 [SMILES: COc1cccc(F)c1CNC(=O)C1(C)COc2c(oc3cc(C(=O)NC4(C)COC4)ccc23)C(=O)N1CC1CC1] | 1H NMR (400 MHz, CDCl3) δ 7.84 (s, 1H), 7.54 (tt, J = 4.6, 2.4 Hz, 2H), 6.88 (s, 1H), 6.82 (dd, J = 15.2, 8.1 Hz, 1H), 6.61 (s, 1H), 6.35 (d, J = 8.4 Hz, 1H), 6.30 (t, J = 8.5 Hz, 1H), 4.92-4.89 (m, 3H), 4.61 (d, J = 6.5 Hz, 2H), 4.60 − 4.51 (m, 1H), 4.13-4.06 (m, 2H), 3.86 − 3.75 (m, 1H), 3.74 (s, 3H), 3.56 (dd, J = 14.9, 5.5 Hz, 1H), 1.85 (s, 3H), 1.81 (s, 3H), 1.09 − 0.96 (m, 1H), 0.64 − 0.36 (m, 4H); LC-MS: 566.2[M + 1]+ |
| 90 | 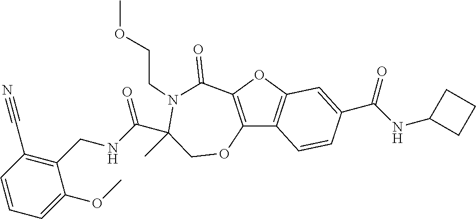 [SMILES: COCCN1C(=O)c2oc3cc(C(=O)NC4CCC4)ccc3c2OCC1(C)C(=O)NCc1c(C#N)cccc1OC] | 1H NMR (400 MHz, CDCl3) δ 7.83 (s, 1H), 7.64 (t, J = 8 Hz, 1H), 7.52 (d, J = 8.2 Hz, 1H), 7.46 (d, J = 8.2 Hz, 1H), 6.86 (t, J = 7.5 Hz, 1H), 6.79 (d, J = 7.3 Hz, 1H), 6.70 (d, J = 8.2 Hz, 1H), 6.26 (d, J = 7.3 Hz, 1H), 4.89 (d, J = 11.7 Hz, 1H), 4.79 (dd, J = 14.1, 7.3 Hz, 1H), 4.63 (dd, J = 16.3, 8.2 Hz, 1H), 4.42 − 4.33 (m, 1H), 4.07 (t, J = 11.8 Hz, 2H), 3.85 − 3.81 (m, 1H), 3.76 (s, 3H), 3.68 − 3.48 (m, 2H), 3.34 (s, 3H), 2.58 − 2.34 (m, 2H), 2.18 − 1.93 (m, 2H), 1.85 − 1.81 (m, 2H), 1.79 (s, 3H). LC-MS: 561.2[M + 1]+ |
| 91 | 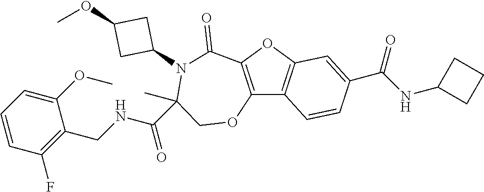 [SMILES: COc1cccc(F)c1CNC(=O)C1(C)COc2c(oc3cc(C(=O)NC4CCC4)ccc23)C(=O)N1[C@H]1C[C@@H](OC)C1] | 1H NMR (400 MHz, DMSO) δ 8.74 (d, J = 7.5 Hz, 1H), 8.32 (s, 1H), 8.04 (s, 1H), 7.82 (dd, J = 8.3, 1.2 Hz, 1H), 7.75 (d, J = 8.3 Hz, 1H), 7.32 (dd, J = 15.4, 8.2 Hz, 1H), 6.87 (d, J = 8.4 Hz, 1H), 6.80 (t, J = 8.8 Hz, 1H), 4.55 (d, J = 12.7 Hz, 1H), 4.50-4.36 (m, 2H), 4.33 (d, J = 12.7 Hz, 1H), 4.29- 4.21 (m, 1H), 3.82 (s, 3H), 3.55 − 3.42 (m, 1H), 3.28 − 3.23 (m, 1H), 3.11 (s, 3H), 2.88- 2.63 (m, 2H), 2.37 − 2.17 (m, 4H), 2.17 − 1.99 (m, 2H), 1.80 − 1.61 (m, 2H), 1.40 (s, 3H). LC-MS: 580.20[M + 1]+ |
| 92 | 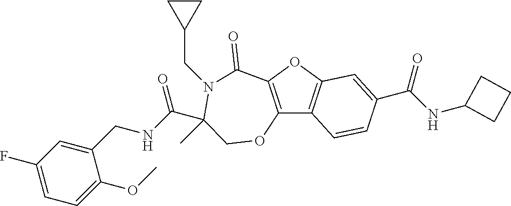 [SMILES: COc1ccc(F)cc1CNC(=O)C1(C)COc2c(oc3cc(C(=O)NC4CCC4)ccc23)C(=O)N1CC1CC1] | 1H NMR (400 MHz, CDCl3) δ 7.83 (s, 1H), 7.52 (d, J = 8.2 Hz, 1H), 7.50 − 7.46 (m, 1H), 7.13 − 7.05 (m, 1H), 6.70 − 6.61 (m, 1H), 6.45-6.37 (m, 2H), 6.29 (d, J = 7.5 Hz, 1H), 4.90 (d, J = 11.9 Hz, 1H), 4.70 − 4.57 (m, 1H), 4.40-4.30 (m, 1H), 4.11 − 3.99 (m, 2H), 3.96 − 3.78 (m, 1H), 3.75 (s, 3H), 3.56 (dd, J = 14.9, 5.5 Hz, 1H), 2.59 − 2.40 (m, 2H), 2.13 − 1.94 (m, 2H), 1.86 (s, 3H), 1.85 − 1.72 (m, 2H), 1.13 − 0.99 (m, 1H), 0.71 − 0.55 (m, 2H), 0.54-0.43 (m, 2H). LC-MS: 550.20[M + 1]+ |
| 93 | 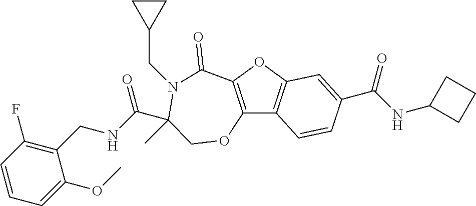 [SMILES: COc1cccc(F)c1CNC(=O)C1(C)COc2c(oc3cc(C(=O)NC4CCC4)ccc23)C(=O)N1CC1CC1] | 1H NMR (400 MHz, CDCl3) δ 7.84 (s, 1H), 7.55 (d, J = 8.1 Hz, 1H), 7.51 (dd, J = 8.2, 1.1 Hz, 1H), 6.87 (s, 1H), 6.79 (dd, J = 15.2, 8.2 Hz, 1H), 6.37 − 6.28 (m, 2H), 6.28 − 6.19 (m, 1H), 4.90 (d, J = 11.9 Hz, 1H), 4.71 − 4.54 (m, 2H), 4.15 − 3.98 (m, 2H), 3.88 − 3.76 (m, 1H), 3.74 (s, 3H), 3.55 (dd, J = 14.9, 5.4 Hz, 1H), 2.56 − 2.37 (m, 2H), 2.10 − 1.93 (m, 2H), 1.93 − 1.74 (m, 6H), 1.13 − 0.98 (m, 1H), 0.71 − 0.53 (m, 2H), 0.53 − 0.32 (m, 2H). LC-MS: 550.20[M + 1]+ |
| 94 | 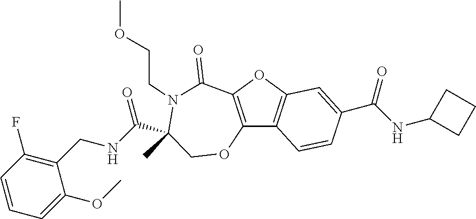 [SMILES: COCCN1C(=O)c2oc3cc(C(=O)NC4CCC4)ccc3c2OC[C@]1(C)C(=O)NCc1c(F)cccc1OC] | 1H NMR (400 MHz, CDCl3) δ 7.83 (s, 1H), 7.57 (d, J = 8.2 Hz, 1H), 7.52 (dd, J = 8.2, 1.1 Hz, 1H), 7.40 − 7.32 (m, 1H), 6.84 (dd, J = 15.1, 8.0 Hz, 1H), 6.41 − 6.33 (m, 1H), 6.33 − 6.23 (m, 2H), 4.87 (d, J = 11.8 Hz, 1H), 4.70 − 4.52 (m, 2H), 4.37 − 4.22 (m, 1H), 4.15 − 4.01 (m, 2H), 3.83 − 3.74 (m, 1H), 3.72 (s, 3H), 3.63 − 3.49 (m, 2H), 3.24 (s, 3H), 2.56 − 2.39 (m, 2H), 2.10 − 1.93 (m, 2H), 1.88 − 1.72 (m, 5H). LC-MS: 554.20[M + 1]+ |
| 95 | 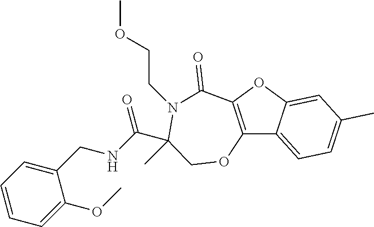 [SMILES: COCCN1C(=O)c2oc3cc(C)ccc3c2OCC1(C)C(=O)NCc1ccccc1OC] | LC-MS: 453.2 [M + 1]⁺; 1H NMR (400 MHz, CDCl3) δ 7.41 (d, J = 8.1 Hz, 1H), 7.36 (s, 1H), 7.20 (s, 1H), 7.02 (d, J = 8.1 Hz, 1H), 6.92 (dd, J = 13.3, 7.1 Hz, 2H), 6.59 (d, J = 8.1 Hz, 1H), 6.50 (t, J = 7.1 Hz, 1H), 4.84 (d, J = 12.0 Hz, 1H), 4.36 (dd, J = 14.4, 6.7 Hz, 1H), 4.27 − 4.00 (m, 3H), 3.84 − 3.76 (m, 1H), 3.75 (s, 3H), 3.65 − 3.53 (m, 2H), 3.23 (s, 3H), 2.48 (s, 3H), 1.76 (s, 3H). |
| 96 | 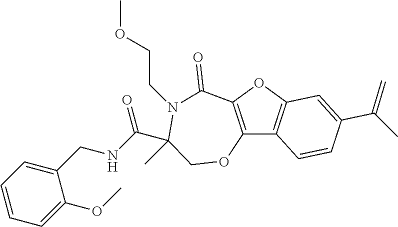 [SMILES: C=C(C)c1ccc2c3c(oc2c1)C(=O)N(CCOC)C(C)(C(=O)NCc1ccccc1OC)CO3] | 1H NMR (400 MHz, CDCl3) δ 7.47-7.45 (m, 2H), 7.42 − 7.37 (m, 1H), 7.34 (dd, J = 8.2, 1.5 Hz, 1H), 6.90-6.86 (m, 2H), 6.57 (d, J = 8.0 Hz, 1H), 6.47 (t, J = 7.2 Hz, 1H), 5.45 (s, 1H), 5.31 − 4.96 (m, 1H), 4.86 (d, J = 11.9 Hz, 1H), 4.37 (dd, J = 14.3, 6.9 Hz, 1H), 4.25-4.21 (m, 1H), 4.20 − 3.99 (m, 2H), 3.82 − 3.77 (m, 1H), 3.76 (s, 3H), 3.63 − 3.51 (m, 2H), 3.25 (s, 3H), 2.21 (s, 3H), 1.77 (s, 3H); LC-MS: 479.2[M + 1]+ |
| 97 | 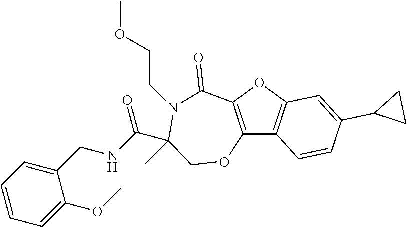 [SMILES: COCCN1C(=O)c2oc3cc(C4CC4)ccc3c2OCC1(C)C(=O)NCc1ccccc1OC] | 1H NMR (400 MHz, CDCl3) δ 7.40-7.36 (m, 2H), 7.08 (s, 1H), 6.99 − 6.81 (m, 3H), 6.58 (d, J = 8.1 Hz, 1H), 6.48 (t, J = 7.3 Hz, 1H), 4.84 (d, J = 12.0 Hz, 1H), 4.36 (dd, J = 14.3, 6.7 Hz, 1H), 4.29 − 3.98 (m, 3H), 3.87 − 3.75 (m, 1H), 3.75 (s, 3H), 3.60-3.54 (m, 2H), 3.23 (s, 3H), 2.09 − 1.90 (m, 1H), 1.75 (s, 3H), 1.17 − 0.90 (m, 2H), 0.78-0.74 (m, 2H). LC-MS: 479.2[M + 1]+ |
| 98 | 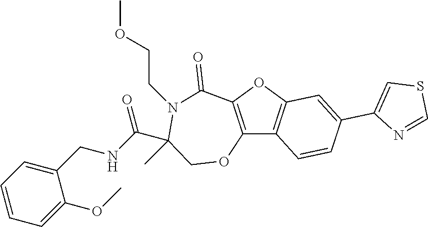 [SMILES: COCCN1C(=O)c2oc3cc(-c4cscn4)ccc3c2OCC1(C)C(=O)NCc1ccccc1OC] | 1H NMR (400 MHz, CDCl3) δ 8.93 (d, J = 1.9 Hz, 1H), 7.99 (s, 1H), 7.77 (dd, J = 8.3, 1.2 Hz, 1H), 7.62 (d, J = 1.9 Hz, 1H), 7.56 (d, J = 8.3 Hz, 1H), 7.42 (t, J = 5.7 Hz, 1H), 6.91 (d, J = 7.2 Hz, 1H), 6.85 (t, J = 7.4 Hz, 1H), 6.58 (d, J = 8.2 Hz, 1H), 6.47 (t, J = 7.3 Hz, 1H), 4.88 (d, J = 11.9 Hz, 1H), 4.39 (dd, J = 14.3, 7.0 Hz, 1H), 4.29-4.27 (m, 1H), 4.19 − 4.03 (m, 2H), 3.84 − 3.77 (m, 1H), 3.77 (s, 3H), 3.65 − 3.54 (m, 2H), 3.26 (s, 3H), 1.78 (s, 3H). LC-MS: 522.20[M + 1]+ |
| 99 | 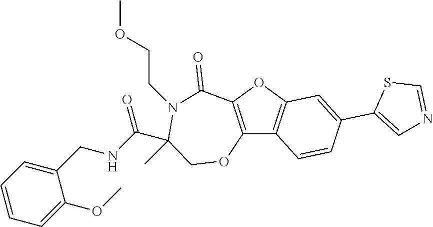 [SMILES: COCCN1C(=O)c2oc3cc(-c4cncs4)ccc3c2OCC1(C)C(=O)NCc1ccccc1OC] | 1H NMR (400 MHz, CDCl3) δ 8.83 (s, 1H), 8.15 (s, 1H), 7.57-7.55 (m, 2H), 7.45-7.41 (m, 2H), 6.87 (dd, J = 14.6, 7.4 Hz, 2H), 6.59 (d, J = 8.1 Hz, 1H), 6.45 (t, J = 7.4 Hz, 1H), 4.89 (d, J = 11.9 Hz, 1H), 4.38 (dd, J = 14.3, 7.1 Hz, 1H), 4.30-4.28 (m, 1H), 4.17 − 4.03 (m, 2H), 3.81-3.80 (m, 1H), 3.79 (s, 3H), 3.72 − 3.53 (m, 2H), 3.26 (s, 3H), 1.79 (s, 3H). LC-MS: 522.20[M + 1]+ |
| 100 | 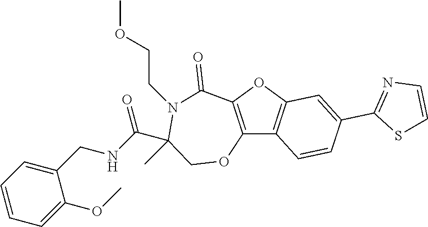 [SMILES: COCCN1C(=O)c2oc3cc(-c4nccs4)ccc3c2OCC1(C)C(=O)NCc1ccccc1OC] | 1H NMR (400 MHz, CDCl3) δ 8.00 (s, 1H), 7.92 (d, J = 3.2 Hz, 1H), 7.80 (dd, J = 8.3, 1.3 Hz, 1H), 7.56 (d, J = 8.3 Hz, 1H), 7.49 − 7.41 (m, 1H), 7.39 (d, J = 3.2 Hz, 1H), 6.90 (d, J = 7.6 Hz, 1H), 6.84 (t, J = 7.7 Hz, 1H), 6.58 (d, J = 8.1 Hz, 1H), 6.44 (t, J = 7.2 Hz, 1H), 4.89 (d, J = 11.8 Hz, 1H), 4.39 (dd, J = 14.2, 7.2 Hz, 1H), 4.30-4.28 (m, 1H), 4.15 − 4.03 (m, 2H), 3.84 − 3.79 (m, 1H), 3.78 (s, 3H), 3.64 − 3.53 (m, 2H), 3.28 (s, 3H), 1.79 (s, 3H). LC-MS: 522.20[M + 1]+ |
| 101 | 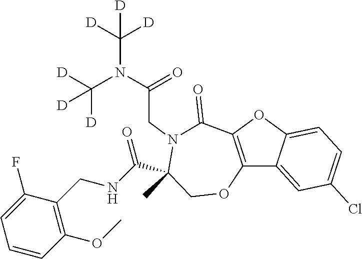 [SMILES: [2H]C([2H])([2H])N(C(=O)CN1C(=O)c2oc3ccc(Cl)cc3c2OC[C@]1(C)C(=O)NCc1c(F)cccc1OC)C([2H])([2H])[2H]] | LC-MS: Calculated Exact Mass = 523.2, Found [M + H]⁺ = 524.2. ¹H NMR (400 MHz, DMSO) δ 9.55 (s, 1H), 7.58 (d, J = 13.5 Hz, 1H), 7.55 (d, J = 8.9 Hz, 1H), 7.49 (dd, J = 8.9, 2.1 Hz, 1H), 7.04 (dd, J = 15.2, 7.8 Hz, 1H), 6.61 (d, J = 8.3 Hz, 1H), 6.38 (s, 1H), 4.84 (d, J = 11.8 Hz, 1H), 4.49 (s, 1H), 4.38 − 4.22 (m, 3H), 3.92 (s, 1H), 3.63 (s, 3H), 1.53 (s, 3H). |
| 102 | 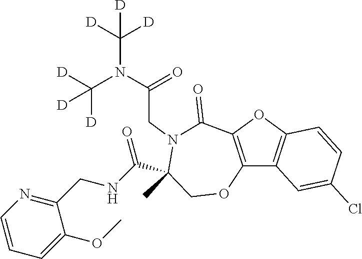 [SMILES: [2H]C([2H])([2H])N(C(=O)CN1C(=O)c2oc3ccc(Cl)cc3c2OC[C@]1(C)C(=O)NCc1ncccc1OC)C([2H])([2H])[2H]] | ¹H NMR (400 MHz, DMSO) δ 9.47 (s, 1H), 7.65 (s, 1H), 7.62 (d, J = 8.9 Hz, 1H), 7.52 (dd, J = 8.9, 2.1 Hz, 2H), 7.22 (d, J = 8.0 Hz, 1H), 7.06 (s, 1H), 4.86 (d, J = 10.4 Hz, 1H), 4.48 (s, 2H), 4.40 − 4.25 (m, 2H), 4.13 (s, 1H), 3.69 (s, 3H), 1.58 (s, 3H); LC-MS: Found [M + H]⁺ = 507.2 |
| 103 | 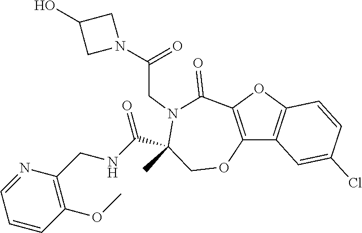 [SMILES: COc1cccnc1CNC(=O)[C@@]1(C)COc2c(oc3ccc(Cl)cc23)C(=O)N1CC(=O)N1CC(O)C1] | ¹H NMR (400 MHz, DMSO) δ 9.27 (s, 1H), 7.65 (s, 1H), 7.63 (d, J = 8.9 Hz, 1H), 7.57 (s, 1H), 7.52 (dd, J = 8.9, 2.2 Hz, 1H), 7.22 (d, J = 8.0 Hz, 1H), 7.07 (s, 1H), 5.82 (t, J = 5.4 Hz, 1H), 4.84 (d, J = 11.8 Hz, 1H), 4.53 (s, 1H), 4.45 − 3.95 (m, 8H), 3.69 (s, 3H), 3.63-3.61 (m, 1H), 1.58 (s, 3H). LC-MS: Found [M + H]⁺ = 529.2 |
| 104 | 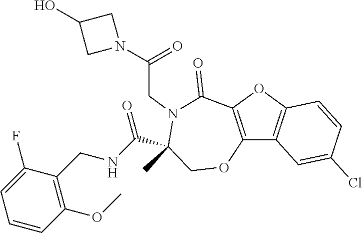 [SMILES: COc1cccc(F)c1CNC(=O)[C@@]1(C)COc2c(oc3ccc(Cl)cc23)C(=O)N1CC(=O)N1CC(O)C1] | ¹H NMR (400 MHz, DMSO) δ 9.31 (s, 1H), 7.64 − 7.53 (m, 2H), 7.49 (dd, J = 8.9, 2.1 Hz, 1H), 7.02 (d, J = 6.2 Hz, 1H), 6.59 (d, J = 7.7 Hz, 1H), 6.38 (s, 1H), 5.82 (dd, J = 7.8, 6.2 Hz, 1H), 4.82 (d, J = 11.8 Hz, 1H), 4.59 − 4.46 (m, 1H), 4.40 (dd, J = 18.1, 9.4 Hz, 1H), 4.35 − 4.03 (m, 5H), 4.03 − 3.83 (m, 2H), 3.67-365 (m, 1H), 3.62 (s, 3H), 1.54 (s, 3H); LC-MS: Found [M + H]⁺ = 546.2 |
| 105 | 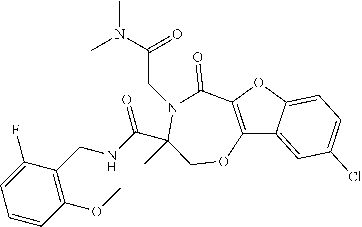 [SMILES: COc1cccc(F)c1CNC(=O)C1(C)COc2c(oc3ccc(Cl)cc23)C(=O)N1CC(=O)N(C)C] | LC-MS: Calculated Exact Mass = 517.2, Found [M + H]⁺ = 518.2. ¹H NMR (400 MHz, DMSO) δ 9.57 (s, 1H), 7.60 (s, 1H), 7.56 (d, J = 8.9 Hz, 1H), 7.49 (dd, J = 8.9, 2.2 Hz, 1H), 7.15 − 6.93 (m, 1H), 6.61 (d, J = 8.2 Hz, 1H), 6.38 (s, 1H), 4.84 (d, J = 11.8 Hz, 1H), 4.50 (m, 1H), 4.38 − 4.18 (m, 3H), 4.01-3.92 (m, 1H), 3.63 (s, 3H), 3.03 (s, 3H), 2.87 (s, 3H), 1.53 (s, 3H). |
| 106 | 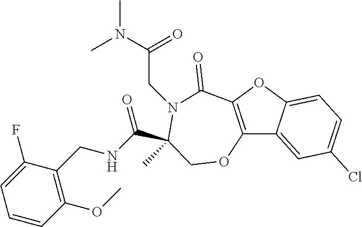 [SMILES: COc1cccc(F)c1CNC(=O)[C@]1(C)COc2c(oc3ccc(Cl)cc23)C(=O)N1CC(=O)N(C)C] | LC-MS: Calculated Exact Mass = 517.2, Found [M + H]⁺ = 518.2. ¹H NMR (400 MHz, DMSO) δ 9.57 (s, 1H), 7.60 (s, 1H), 7.56 (d, J = 8.9 Hz, 1H), 7.49 (dd, J = 8.9, 2.2 Hz, 1H), 7.15 − 6.93 (m, 1H), 6.61 (d, J = 8.2 Hz, 1H), 6.38 (s, 1H), 4.84 (d, J = 11.8 Hz, 1H), 4.50 (m, 1H), 4.38 − 4.18 (m, 3H), 4.01-3.92 (m, 1H), 3.63 (s, 3H), 3.03 (s, 3H), 2.87 (s, 3H), 1.53 (s, 3H) |
| 107 | 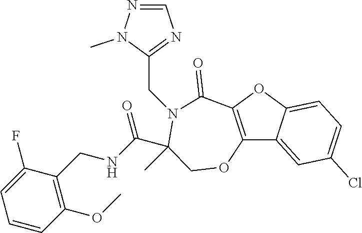 [SMILES: COc1cccc(F)c1CNC(=O)C1(C)COc2c(oc3ccc(Cl)cc23)C(=O)N1Cc1ncnn1C] | LC-MS: Calculated Exact Mass = 527.2, Found [M + H]⁺ = 528.2. ¹H NMR (400 MHz, DMSO) δ 9.30 (s, 1H), 7.66 (d, J = 2.0 Hz, 1H), 7.60 (d, J = 8.9 Hz, 1H), 7.52 (dd, J = 8.9, 2.2 Hz, 1H), 7.50 (s, 1H), 7.16 (dd, J = 15.3, 8.3 Hz, 1H), 6.70 (d, J = 8.4 Hz, 1H), 6.54 (t, J = 8.7 Hz, 1H), 4.93 (d, J = 12.1 Hz, 1H), 4.87 (s, 2H), 4.40 (d, J = 12.1 Hz, 1H), 4.20 (dd, J = 13.7, 5.1 Hz, 1H), 4.13 − 4.03 (m, 1H), 3.86 (s, 3H), 3.60 (s, 3H), 1.66 (s, 3H) |
| 108 | 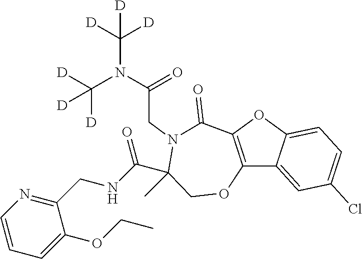 [SMILES: [2H]C([2H])([2H])N(C(=O)CN1C(=O)c2oc3ccc(Cl)cc3c2OCC1(C)C(=O)NCc1ncccc1OCC)C([2H])([2H])[2H]] | LC-MS: Calculated Exact Mass = 521.2, Found [M + H]⁺ = 522.2. ¹H NMR (400 MHz, DMSO) δ 9.42 (s, 1H), 7.63-7.61 (m, 2H), 7.52 (dd, J = 8.9, 2.2 Hz, 2H), 7.18 (d, J = 7.6 Hz, 1H), 7.02 (s, 1H), 4.85 (d, J = 11.2 Hz, 1H), 4.48 (s, 2H), 4.40 − 4.23 (m, 2H), 4.21 − 3.80 (m, 3H), 1.58 (s, 3H), 1.27 (t, J = 6.9 Hz, 3H) |
| 109 | 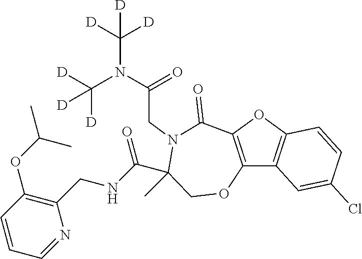 [SMILES: [2H]C([2H])([2H])N(C(=O)CN1C(=O)c2oc3ccc(Cl)cc3c2OCC1(C)C(=O)NCc1ncccc1OC(C)C)C([2H])([2H])[2H]] | ¹H NMR (400 MHz, DMSO) δ 9.31 (s, 1H), 7.66 (s, 1H), 7.63 (d, J = 8.9 Hz, 1H), 7.52 (dd, J = 8.9, 2.2 Hz, 2H), 7.23 (d, J = 8.1 Hz, 1H), 7.04 (s, 1H), 4.86 (d, J = 10.0 Hz, 1H), 4.65 − 4.44 (m, 3H), 4.37 (d, J = 12.3 Hz, 1H), 4.26 (dd, J = 15.8, 6.0 Hz, 1H), 4.15-4.13 (m, 1H), 1.58 (s, 3H), 1.21 (dd, J = 9.6, 6.0 Hz, 6H). LC-MS: Found [M + H]⁺ = 535.2 |
| 110 | 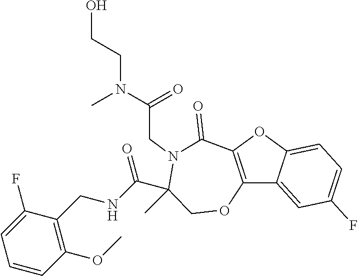 [SMILES: COc1cccc(F)c1CNC(=O)C1(C)COc2c(oc3ccc(F)cc23)C(=O)N1CC(=O)N(C)CCO] | LC-MS: Calculated Exact Mass = 531.2, Found [M + H]⁺ = 532.2. ¹H NMR (400 MHz, DMSO) δ 9.54 (s, 1H), 7.54 (dt, J = 7.6, 3.7 Hz, 1H), 7.45 − 7.21 (m, 2H), 7.02 (dd, J = 15.2, 7.7 Hz, 1H), 6.61 (d, J = 8.3 Hz, 1H), 6.38 (s, 1H), 4.99 (s, 1H), 4.82 (d, J = 11.6 Hz, 1H), 4.36 (ddd, J = 80.0, 54.0, 18.7 Hz, 5H), 3.89 (d, J = 10.8 Hz, 1H), 3.63 (s, 3H), 3.61 − 3.43 (m, 3H), 3.08 (s, 1H), 2.88 (s, 2H), 1.53 (s, 1H), 1.50 (s, 2H) |
| 111 | 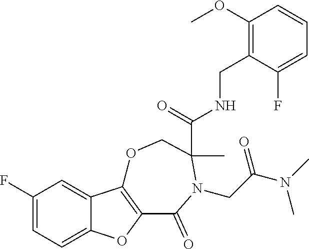 [SMILES: COc1cccc(F)c1CNC(=O)C1(C)COc2c(oc3ccc(F)cc23)C(=O)N1CC(=O)N(C)C] | LC-MS: Calculated Exact Mass = 502.2, Found [M + H]⁺ = 501.2. ¹H NMR (400 MHz, DMSO) δ 9.54 (s, 1H), 7.55 (dd, J = 9.0, 3.9 Hz, 1H), 7.41 − 7.27 (m, 2H), 7.03 (dd, J = 15.2, 7.8 Hz, 1H), 6.61 (d, J = 8.3 Hz, 1H), 6.40 (s, 1H), 4.83 (d, J = 11.7 Hz, 1H), 4.64 − 4.21 (m, 4H), 3.93-3.91 (m, 1H), 3.63 (s, 3H), 3.03 (s, 3H), 2.86 (s, 3H), 1.53 (s, 3H) |
| 112 | 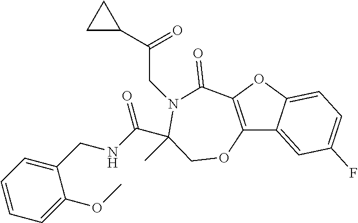 [SMILES: COc1ccccc1CNC(=O)C1(C)COc2c(oc3ccc(F)cc23)C(=O)N1CC(=O)C1CC1] | LC-MS: Calculated Exact Mass = 480.2, Found [M + H]⁺ = 481.2. ¹H NMR (400 MHz, DMSO) δ 8.75 (s, 1H), 7.67 (dd, J = 9.1, 3.9 Hz, 1H), 7.49 (dd, J = 8.1, 2.6 Hz, 1H), 7.41 (td, J = 9.3, 2.7 Hz, 1H), 7.03 (t, J = 7.4 Hz, 1H), 6.82 (d, J = 8.2 Hz, 1H), 6.53 (s, 1H), 6.23 (s, 1H), 4.94 (d, J = 12.2 Hz, 1H), 4.84 (d, J = 18.3 Hz, 1H), 4.55-4.51 (m, 1H), 4.41 (d, J = 12.3 Hz, 1H), 4.25 (dd, J = 15.9, 6.4 Hz, 1H), 4.12 − 3.95 (m, 1H), 3.70 (s, 3H), 2.19-2.17 (m, 1H), 1.56 (s, 3H), 1.06 − 0.73 (m, 4H) |
| 113 | 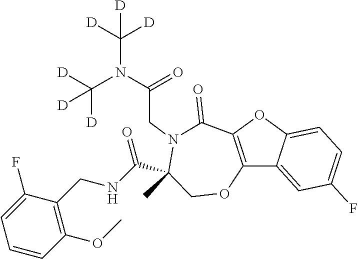 | LC-MS: Calculated Exact Mass = 579 Found [M + H]⁺ = 580.2; ¹H NMR (400 MHz, DMSO) δ 9.41 (s, 1H), 7.62 (dd, J = 9.1, 3.9 Hz, 1H), 7.56 (s, 1H), 7.41 (d, J = 7.5 Hz, 1H), 7.35 (dt, J = 9.2, 4.6 Hz, 1H), 7.22 (d, J = 8.2 Hz, 1H), 7.06 (s, 1H), 4.84 (d, J = 11.4 Hz, 1H), 4.47 (s, 2H), 4.41 − 4.25 (m, 2H), 4.15 (s, 1H), 3.69 (s, 3H), 1.57 (s, 3H) |
| 114 | 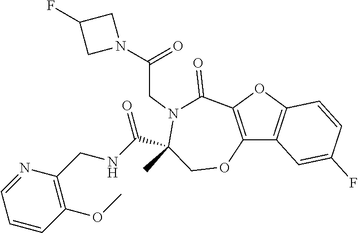 [SMILES: COc1cccnc1CNC(=O)[C@@]1(C)COc2c(oc3ccc(F)cc23)C(=O)N1CC(=O)N1CC(F)C1] | ¹H NMR (400 MHz, DMSO) δ 9.13 (s, 1H), 7.63 (dd, J = 9.1, 3.9 Hz, 1H), 7.60 (s, 1H), 7.43 (d, J = 7.9 Hz, 1H), 7.37 (td, J = 9.3, 2.7 Hz, 1H), 7.23 (d, J = 8.1 Hz, 1H), 7.08 (s, 1H), 5.62 − 5.32 (m, 1H), 4.84 (d, J = 11.7 Hz, 1H), 4.67 − 4.53 (m, 1H), 4.46 − 4.03 (m, 7H), 4.02 − 3.85 (m, 1H), 3.70 (s, 3H), 1.59 (s, 3H); LC-MS: Found [M + H]⁺ = 515.2 |
| 115 | 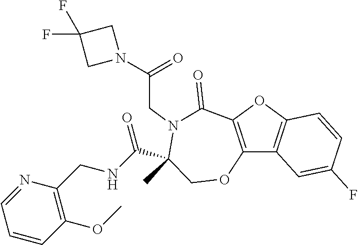 [SMILES: COc1cccnc1CNC(=O)[C@@]1(C)COc2c(oc3ccc(F)cc23)C(=O)N1CC(=O)N1CC(F)(F)C1] | ¹H NMR (400 MHz, DMSO) δ 8.98 (s, 1H), 7.64 (dd, J = 9.1, 3.9 Hz, 2H), 7.48 − 7.42 (m, 1H), 7.37 (td, J = 9.3, 2.7 Hz, 1H), 7.23 (d, J = 8.1 Hz, 1H), 7.09 (d, J = 4.7 Hz, 1H), 4.85 (d, J = 12.2 Hz, 1H), 4.77 (t, J = 12.2 Hz, 2H), 4.47 − 4.28 (m, 5H), 4.18 (s, 2H), 3.71 (s, 3H), 1.61 (s, 3H). LC-MS: Found [M + H]⁺ = 533.2 |
| 116 | 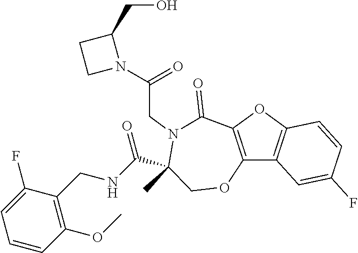 [SMILES: COc1cccc(F)c1CNC(=O)[C@@]1(C)COc2c(oc3ccc(F)cc23)C(=O)N1CC(=O)N1CC[C@H]1CO] | ¹H NMR (400 MHz, DMSO) δ 9.57 (s, 0.5H), 9.21 (s, 0.5H), 7.55 (td, J = 8.6, 3.9 Hz, 1H), 7.33 (tt, J = 6.8, 4.6 Hz, 2H), 7.01 (dd, J = 15.5, 7.1 Hz, 1H), 6.58 (dd, J = 19.2, 7.8 Hz, 1H), 6.37 (s, 1H), 5.25 − 4.72 (m, 2H), 4.66 − 4.06 (m, 7H), 3.97 − 3.70 (m, 3H), 3.62 (d, J = 7.8 Hz, 3H), 2.34-2.33 (m, 1H), 2.17-2.13 (m, 0.5H), 2.03-2.00 (m, 0.5H), 1.54 (s, 3H); LC-MS: Found [M + H]⁺ = 544.2 |
| 117 | 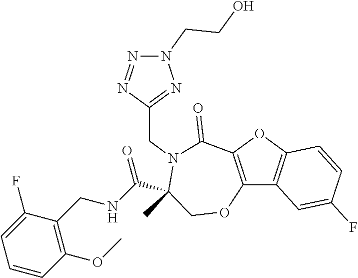 [SMILES: COc1cccc(F)c1CNC(=O)[C@@]1(C)COc2c(oc3ccc(F)cc23)C(=O)N1Cc1nnn(CCO)n1] | ¹H NMR (400 MHz, DMSO) δ 8.31 (s, 1H), 7.61 (dd, J = 9.0, 3.9 Hz, 1H), 7.37 (ddd, J = 11.8, 8.4, 3.7 Hz, 2H), 7.14 (d, J = 8.0 Hz, 1H), 6.70 (d, J = 8.2 Hz, 1H), 6.53 (s, 1H), 5.23 (d, J = 16.6 Hz, 1H), 5.03 (t, J = 5.5 Hz, 1H), 4.91 (d, J = 12.7 Hz, 1H), 4.68 − 4.60 (m, 3H), 4.39 (d, J = 12.5 Hz, 1H), 4.27 (dd, J = 14.0, 4.9 Hz, 1H), 4.17-4.13 (m, 1H), 3.87 (dd, J = 10.6, 5.4 Hz, 2H), 3.70 (d, J = 14.7 Hz, 4H), 1.63 (s, 3H). LC-MS: Found [M + H]⁺ = 543.2 |
| 118 | 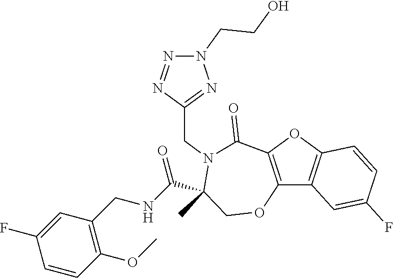 [SMILES: COc1ccc(F)cc1CNC(=O)[C@@]1(C)COc2c(oc3ccc(F)cc23)C(=O)N1Cc1nnn(CCO)n1] | ¹H NMR (400 MHz, DMSO) δ 8.64 (t, J = 5.9 Hz, 1H), 7.64 (dd, J = 9.1, 3.9 Hz, 1H), 7.43 (dd, J = 8.1, 2.6 Hz, 1H), 7.37 (td, J = 9.3, 2.7 Hz, 1H), 6.86 (d, J = 4.6 Hz, 2H), 6.54 (s, 1H), 5.38 (d, J = 17.1 Hz, 1H), 5.05 (dd, J = 11.0, 5.5 Hz, 2H), 4.80 (d, J = 16.7 Hz, 1H), 4.70 − 4.60 (m, 2H), 4.46 (d, J = 12.4 Hz, 1H), 4.17 (ddd, J = 36.6, 16.0, 5.6 Hz, 2H), 3.88 (dd, J = 10.7, 5.3 Hz, 2H), 3.71 (s, 3H), 1.77 (s, 3H); LC-MS: Found [M + H]⁺ = 543.2 |
| 119 | 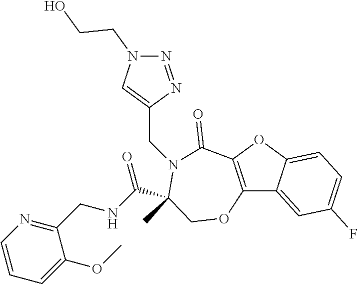 [SMILES: COc1cccnc1CNC(=O)[C@@]1(C)COc2c(oc3ccc(F)cc23)C(=O)N1Cc1cn(CCO)nn1] | LC-MS: Calculated Exact Mass = 524.2, Found [M + H]⁺ = 525.3. ¹H NMR (400 MHz, DMSO) δ 8.34 (s, 1H), 7.94 (s, 1H), 7.77 (s, 1H), 7.66 (dd, J = 9.1, 3.9 Hz, 1H), 7.46 (dd, J = 8.1, 2.5 Hz, 1H), 7.37 (td, J = 9.3, 2.7 Hz, 1H), 7.30 (d, J = 7.7 Hz, 1H), 7.17 (dd, J = 8.2, 4.7 Hz, 1H), 5.05 (d, J = 16.1 Hz, 1H), 4.99 (t, J = 5.3 Hz, 1H), 4.87 (d, J = 12.4 Hz, 1H), 4.56 (d, J = 16.1 Hz, 1H), 4.41 (d, J = 12.6 Hz, 1H), 4.37 − 4.14 (m, 4H), 3.75 (s, 3H), 3.75 − 3.71 (m, 2H), 1.65 (s, 3H). |
| 120 | 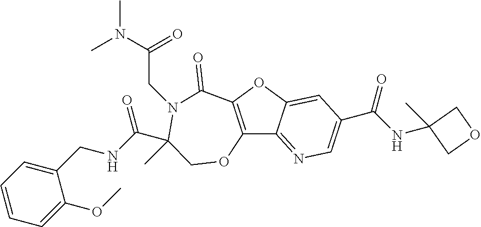 | LC-MS: Calculated Exact Mass = 490.2, Found [M + H]⁺ = 491.3. ¹H NMR (400 MHz, DMSO) δ 9.71 (s, 1H), 9.20 (s, 1H), 9.04 (d, J = 1.7 Hz, 1H), 8.43 (d, J = 1.7 Hz, 1H), 6.97 (t, J = 7.6 Hz, 1H), 6.81 (d, J = 8.1 Hz, 1H), 6.47 (s, 1H), 6.19 (s, 1H), 4.99 (d, J = 11.9 Hz, 1H), 4.78 (d, J = 6.3 Hz, 2H), 4.54- 4.42 (m, 5H), 4.22 (dd, J = 15.7, 6.6 Hz, 1H), 3.95 (dd, J = 15.4, 4.8 Hz, 1H), 3.69 (s, 3H), 3.07 (s, 3H), 2.89 (s, 3H), 1.67 (s, 3H), 1.64 (s, 2H) |
| 121 | 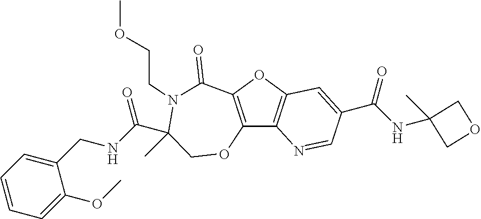 [SMILES: COCCN1C(=O)c2oc3cc(C(=O)NC4(C)COC4)cnc3c2OCC1(C)C(=O)NCc1ccccc1OC] | ¹H NMR (400MHz, CDCl3) δ 8.86 (d, J = 1.6 Hz, 1H), 8.08 (d, J = 1.6 Hz, 1H), 7.40 (d, J = 5.3 Hz, 1H), 7.26 (s, 1H), 7.04 (s, 1H), 6.94 (dd, J = 17.9, 7.9 Hz, 2H), 6.66 (d, J = 8.1 Hz, 1H), 6.50 (s, 1H), 4.93 (d, J = 6.5 Hz, 2H), 4.90 (s, 1H), 4.61 (d, J = 6.6 Hz, 2H), 4.33 (dd, J = 14.2, 6.7 Hz, 1H), 4.27 − 4.07 (m, 3H), 3.84-3.81 (m, 1H), 3.79 (s, 3H), 3.69 − 3.52 (m, 2H), 1.82 (s, 3H), 1.77 (s, 3H); LC-MS: Found [M + H]⁺ = 553 |
| 122 | 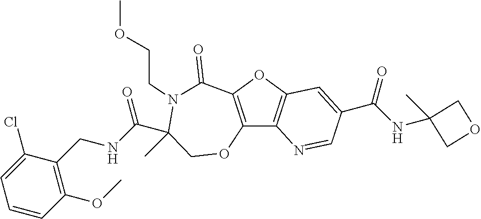 [SMILES: COCCN1C(=O)c2oc3cc(C(=O)NC4(C)COC4)cnc3c2OCC1(C)C(=O)NCc1c(Cl)cccc1OC] | ¹H NMR (400 MHz, DMSO) δ 9.29 (s, 1H), 9.03 (d, J = 1.6 Hz, 1H), 8.45 (d, J = 1.6 Hz, 1H), 7.94 (s, 1H), 7.19 (t, J = 8.1 Hz, 1H), 6.97 − 6.83 (m, 2H), 4.90 (d, J = 11.1 Hz, 1H), 4.78 (d, J = 6.3 Hz, 2H), 4.42 (d, J = 6.4 Hz, 2H), 4.36 (dd, J = 13.4, 4.8 Hz, 2H), 4.26 (d, J = 12.3 Hz, 1H), 3.75 (s, 3H), 3.66 (s, 2H), 3.51-3.48 (m, 2H), 3.20 (s, 3H), 1.66 (s, 3H), 1.64 (s, 3H). LC-MS: 587.2[M + 1]+ |
| 123 | 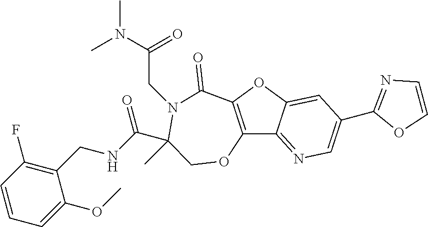 [SMILES: COc1cccc(F)c1CNC(=O)C1(C)COc2c(oc3cc(-c4ncco4)cnc23)C(=O)N1CC(=O)N(C)C] | LC-MS: Calculated Exact Mass = 551.2, Found [M + H]⁺ = 552.2. ¹H NMR (400 MHz, DMSO) δ 9.62 (s, 1H), 9.12 (d, J = 1.6 Hz, 1H), 8.46 (d, J = 1.5 Hz, 1H), 8.39 (d, J = 0.7 Hz, 1H), 7.52 (d, J = 0.7 Hz, 1H), 6.93-6.91 (m, 1H), 6.64 (d, J = 8.1 Hz, 1H), 6.33-6.31 (m, 1H), 4.90 (d, J = 11.9 Hz, 1H), 4.55-4.51 (m, 1H), 4.43 − 4.26 (m, 3H), 3.89-3.84 (m, 1H), 3.66 (s, 3H), 3.05 (s, 3H), 2.89 (s, 3H), 1.57 (s, 3H). |
| 124 | 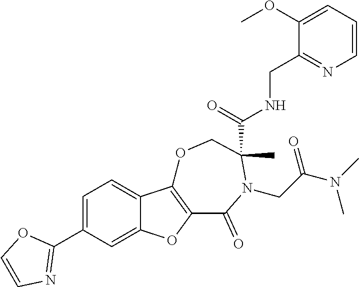 [SMILES: COc1cccnc1CNC(=O)[C@@]1(C)COc2c(oc3cc(-c4ncco4)ccc23)C(=O)N1CC(=O)N(C)C] | LC-MS: Calculated Exact Mass = 533.2, Found [M + H]⁺ = 534.2. ¹H NMR (400 MHz, DMSO) δ 9.45 (s, 1H), 8.30 (d, J = 0.6 Hz, 1H), 8.09 (s, 1H), 7.92 (dd, J = 8.3, 1.0 Hz, 1H), 7.76 (d, J = 8.2 Hz, 1H), 7.55 (s, 1H), 7.46 (d, J = 0.7 Hz, 1H), 7.22 (d, J = 8.1 Hz, 1H), 7.02 (s, 1H), 4.88 (d, J = 11.9 Hz, 1H), 4.50 (s, 2H), 4.38 (d, J = 11.4 Hz, 1H), 4.31 (dd, J = 15.6, 6.2 Hz, 1H), 4.14 (s, 1H), 3.69 (s, 3H), 3.06 (s, 3H), 2.86 (s, 3H), 1.59 (s, 3H) |
| 125 | 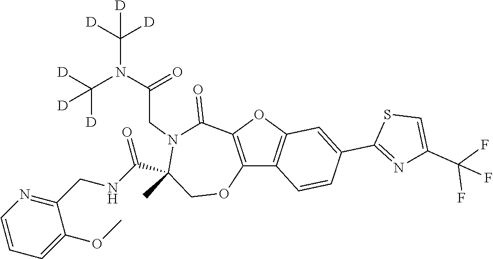 [SMILES: [2H]C([2H])([2H])N(C(=O)CN1C(=O)c2oc3cc(-c4nc(C(F)(F)F)cs4)ccc3c2OC[C@]1(C)C(=O)NCc1ncccc1OC)C([2H])([2H])[2H]] | ¹H NMR (400 MHz, DMSO) δ 9.46 (s, 1H), 8.63 (s, 1H), 8.19 (s, 1H), 7.93 (dd, J = 8.3, 1.1 Hz, 1H), 7.76 (d, J = 8.2 Hz, 1H), 7.56 (s, 1H), 7.23 (d, J = 8.1 Hz, 1H), 7.02 (s, 1H), 4.88 (d, J = 10.7 Hz, 1H), 4.49 (s, 2H), 4.39 (d, J = 12.2 Hz, 1H), 4.33 (d, J = 6.2 Hz, 1H), 4.14 (s, 1H), 3.69 (s, 3H), 1.59 (s, 3H); LC-MS: Found [M + H]⁺ = 624.2 |
| 126 | 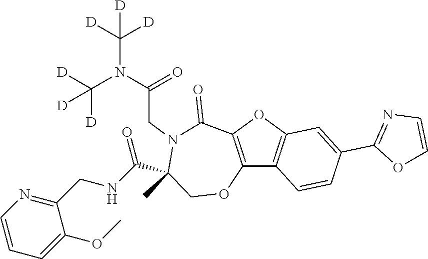 [SMILES: [2H]C([2H])([2H])N(C(=O)CN1C(=O)c2oc3cc(-c4ncco4)ccc3c2OC[C@]1(C)C(=O)NCc1ncccc1OC)C([2H])([2H])[2H]] | ¹H NMR (400 MHz, DMSO) δ 9.46 (s, 1H), 8.30 (d, J = 0.7 Hz, 1H), 8.09 (s, 1H), 7.92 (dd, J = 8.3, 1.1 Hz, 1H), 7.76 (d, J = 8.2 Hz, 1H), 7.54 (s, 1H), 7.46 (d, J = 0.7 Hz, 1H), 7.22 (d, J = 7.9 Hz, 1H), 7.02 (s, 1H), 4.88 (d, J = 11.9 Hz, 1H), 4.49 (s, 2H), 4.38 (d, J = 11.9 Hz, 1H), 4.31 (dd, J = 15.5, 6.2 Hz, 1H), 4.14 (s, 1H), 3.68 (s, 3H), 1.59 (s, 3H). LC-MS: Found [M + H]⁺ = 540.2 |
| 127 | 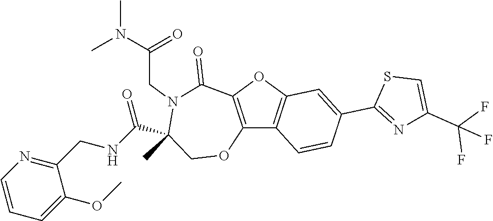 [SMILES: COc1cccnc1CNC(=O)[C@@]1(C)COc2c(oc3cc(-c4nc(C(F)(F)F)cs4)ccc23)C(=O)N1CC(=O)N(C)C] | ¹H NMR (400 MHz, DMSO) δ 9.45 (s, 1H), 8.63 (d, J = 0.8 Hz, 1H), 8.19 (s, 1H), 7.93 (dd, J = 8.3, 1.2 Hz, 1H), 7.76 (d, J = 8.2 Hz, 1H), 7.56 (s, 1H), 7.23 (d, J = 8.0 Hz, 1H), 7.02 (s, 1H), 4.89 (d, J = 11.3 Hz, 1H), 4.50 (s, 2H), 4.39 (d, J = 11.6 Hz, 1H), 4.31 (dd, J = 15.5, 6.2 Hz, 1H), 4.15 (s, 1H), 3.70 (s, 3H), 3.07 (s, 3H), 2.86 (s, 3H), 1.60 (s, 3H). LC-MS: Found [M + H]⁺ = 618.2 |
| 128 | 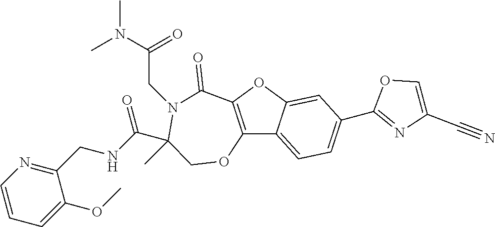 [SMILES: COc1cccnc1CNC(=O)C1(C)COc2c(oc3cc(-c4nc(C#N)co4)ccc23)C(=O)N1CC(=O)N(C)C] | LC-MS: Calculated Exact Mass = 558.2, Found [M + H]⁺ = 559.2. ¹H NMR (400 MHz, DMSO) δ 9.47 (s, 1H), 9.28 (s, 1H), 8.18 (s, 1H), 7.92 (dd, J = 8.3, 1.0 Hz, 1H), 7.80 (d, J = 8.2 Hz, 1H), 7.51 (s, 1H), 7.21 (d, J = 8.1 Hz, 1H), 7.01 (s, 1H), 4.89 (d, J = 11.8 Hz, 1H), 4.50 (s, 2H), 4.39 (d, J = 11.7 Hz, 1H), 4.32 (dd, J = 15.5, 6.3 Hz, 1H), 4.19 − 4.06 (m, 1H), 3.68 (s, 3H), 3.07 (s, 3H), 2.86 (s, 3H), 1.60 (s, 3H) |
| 129 | 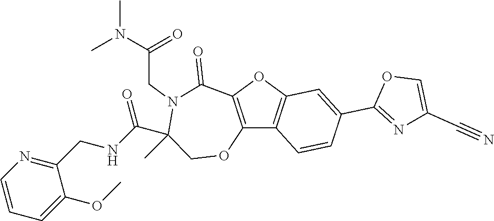 [SMILES: COc1cccnc1CNC(=O)C1(C)COc2c(oc3cc(-c4nc(C#N)co4)ccc23)C(=O)N1CC(=O)N(C)C] | ¹H NMR (400 MHz, DMSO) δ 9.46 (s, 1H), 9.28 (s, 1H), 8.19 (s, 1H), 7.93 (dd, J = 8.3, 1.1 Hz, 1H), 7.80 (d, J = 8.2 Hz, 1H), 7.51 (s, 1H), 7.21 (d, J = 8.0 Hz, 1H), 7.01 (s, 1H), 4.89 (d, J = 11.1 Hz, 1H), 4.50 (s, 2H), 4.39 (d, J = 11.8 Hz, 1H), 4.32 (dd, J = 15.5, 6.3 Hz, 1H), 4.13 (s, 1H), 3.68 (s, 3H), 3.07 (s, 3H), 2.86 (s, 3H), 1.59 (s, 3H). LC-MS: Found [M + H]⁺ = 559.2 |
| 130 | 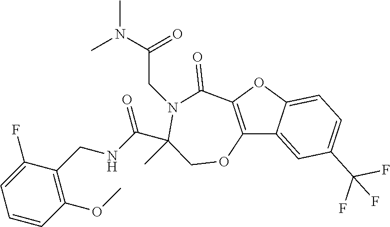 [SMILES: COc1cccc(F)c1CNC(=O)C1(C)COc2c(oc3ccc(C(F)(F)F)cc23)C(=O)N1CC(=O)N(C)C] | LC-MS: Calculated Exact Mass = 551.2, Found [M + H]⁺ = 552.2. ¹H NMR (400 MHz, DMSO) δ 9.59 (s, 1H), 7.90 (s, 1H), 7.80 (dd, J = 8.9, 1.6 Hz, 1H), 7.74 (d, J = 8.8 Hz, 1H), 6.97 (d, J = 7.7 Hz, 1H), 6.57 (d, J = 8.3 Hz, 1H), 6.29 (s, 1H), 4.87 (d, J = 12.0 Hz, 1H), 4.52 (s, 1H), 4.31 (dt, J = 13.7, 11.3 Hz, 3H), 3.89 (s, 1H), 3.62 (s, 3H), 3.04 (s, 3H), 2.88 (s, 3H), 1.55 (s, 3H) |
| 131 | 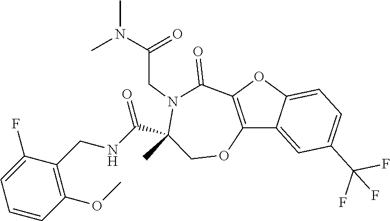 [SMILES: COc1cccc(F)c1CNC(=O)[C@@]1(C)COc2c(oc3ccc(C(F)(F)F)cc23)C(=O)N1CC(=O)N(C)C] | ¹H NMR (400 MHz, DMSO) δ 9.59 (s, 1H), 7.90 (s, 1H), 7.80 (dd, J = 8.9, 1.6 Hz, 1H), 7.74 (d, J = 8.8 Hz, 1H), 6.97 (d, J = 7.7 Hz, 1H), 6.57 (d, J = 8.3 Hz, 1H), 6.29 (s, 1H), 4.87 (d, J = 12.0 Hz, 1H), 4.52 (s, 1H), 4.31 (dt, J = 13.7, 11.3 Hz, 3H), 3.89 (s, 1H), 3.62 (s, 3H), 3.04 (s, 3H), 2.88 (s, 3H), 1.55 (s, 3H) LC-MS: Found [M + H]⁺ = 552.2 |
| 132 | 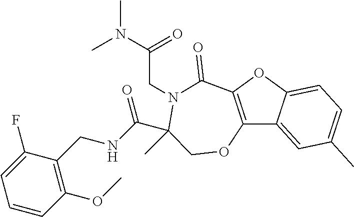 [SMILES: COc1cccc(F)c1CNC(=O)C1(C)COc2c(oc3ccc(C)cc23)C(=O)N1CC(=O)N(C)C] | ¹H NMR (400 MHz, DMSO) δ 9.45 (s, 1H), 7.38 (d, J = 8.5 Hz, 1H), 7.35 (s, 1H), 7.29 (d, J = 8.6 Hz, 1H), 7.08 (dd, J = 15.3, 7.7 Hz, 1H), 6.63 (d, J = 8.4 Hz, 1H), 6.45 (s, 1H), 4.82 (d, J = 11.7 Hz, 1H), 4.45 (s, 1H), 4.35 − 4.15 (m, 3H), 4.00 (s, 1H), 3.61 (s, 3H), 3.02 (s, 3H), 2.84 (s, 3H), 2.40 (s, 3H), 1.51 (s, 3H) LC-MS: Found [M + H]⁺ = 498.2 |
| 133 | 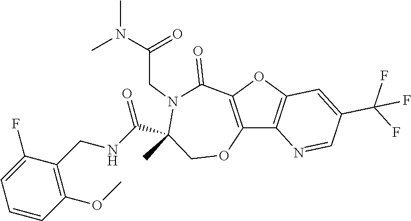 [SMILES: COc1cccc(F)c1CNC(=O)[C@@]1(C)COc2c(oc3cc(C(F)(F)F)cnc23)C(=O)N1CC(=O)N(C)C] | ¹H NMR (400 MHz, DMSO) δ 9.71 (s, 1H), 8.89 (s, 1H), 8.57 (s, 1H), 6.87 (d, J = 7.0 Hz, 1H), 6.61 (d, J = 8.2 Hz, 1H), 6.24 (s, 1H), 4.89 (d, J = 11.8 Hz, 1H), 4.58 (s, 1H), 4.47 − 4.31 (m, 3H), 3.79 (d, J = 6.5 Hz, 1H), 3.69 (d, J = 6.8 Hz, 3H), 3.06 (s, 3H), 2.91 (s, 3H), 1.57 (s, 3H). LC-MS: Found [M + H]⁺ = 553.2 |
| 134 | 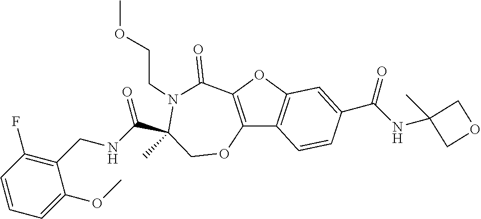 [SMILES: COCCN1C(=O)c2oc3cc(C(=O)NC4(C)COC4)ccc3c2OC[C@@]1(C)C(=O)NCc1c(F)cccc1OC] | ¹H NMR (400 MHz, CDCl3) δ 7.83 (s, 1H), 7.61 (d, J = 8.2 Hz, 1H), 7.53 (d, J = 7.9 Hz, 1H), 7.39 − 7.30 (m, 1H), 6.94 − 6.79 (m, 1H), 6.48 (s, 1H), 6.44 − 6.27 (m, 2H), 4.96 − 4.82 (m, 3H), 4.66 − 4.50 (m, 3H), 4.33 − 4.19 (m, 1H), 4.17 − 4.02 (m, 2H), 3.85 − 3.75 (m, 1H), 3.73 (s, 3H), 3.63 − 3.49 (m, 2H), 3.23 (s, 3H), 1.81 (s, 3H), 1.77 (s, 3H). LC-MS: 570.20[M + 1]+ |
| 135 | 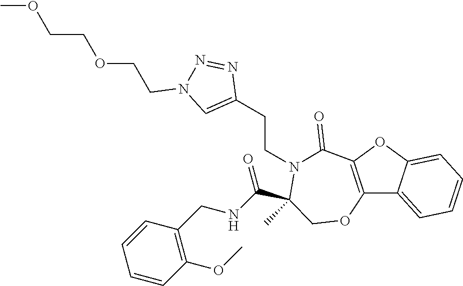 [SMILES: COCCOCCn1cc(CCN2C(=O)c3oc4ccccc4c3OC[C@@]2(C)C(=O)NCc2ccccc2OC)nn1] | ¹H NMR (400 MHz, CDCl3) δ 7.61 (s, 1H), 7.51 (d, J = 7.9 Hz, 1H), 7.46 − 7.37 (m, 2H), 7.20 (ddd, J = 8.0, 5.1, 3.0 Hz, 1H), 7.04 (t, J = 5.8 Hz, 1H), 6.98 (d, J = 7.2 Hz, 1H), 6.93 (t, J = 7.7 Hz, 1H), 6.54 (dd, J = 12.2, 7.8 Hz, 2H), 4.81 (d, J = 12.1 Hz, 1H), 4.62 − 4.45 (m, 2H), 4.40 (dd, J = 14.3, 6.8 Hz, 1H), 4.21 (dd, J = 14.2, 5.0 Hz, 1H), 4.08 (d, J = 12.1 Hz, 1H), 4.05 − 3.94 (m, 1H), 3.94 − 3.78 (m, 3H), 3.70 (s, 3H), 3.64 − 3.55 (m, 2H), 3.52 − 3.48 (m, 2H), 3.36 (s, 3H), 3.23-3.16 (m, 1H), 3.11 − 3.01 (m, 1H), 1.79 (s, 3H). LC-MS: 478.2[M + 1]+ |
| 136 | 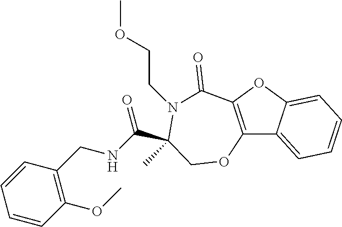 | ¹H NMR (400 MHz, DMSO) δ 7.60 (d, J = 8.4 Hz, 1H), 7.51 (t, J = 7.8 Hz, 1H), 7.29 (t, J = 7.4 Hz, 1H), 7.17 − 7.15 (m, 4H), 4.96 − 4.95 (m, 1H), 4.79 (d, J = 15.5 Hz, 1H), 4.57 − 4.54 (m, 2H), 3.92 (d, J = 12.0 Hz, 2H), 3.47 − 3.42 (m, 3H), 3.19 − 3.18 (m, 4H), 2.76 − 2.75 (m, 2H), 1.58 (s, 3H). LC- MS: 435.04[M + 1]+ |
| 137 | 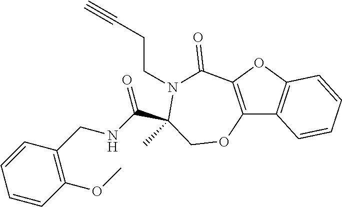 [SMILES: C#CCCN1C(=O)c2oc3ccccc3c2OC[C@@]1(C)C(=O)NCc1ccccc1OC] | ¹H NMR (400 MHz, CDCl3) δ 7.52 (d, J = 7.9 Hz, 1H), 7.45 − 7.40 (m, 2H), 7.21 (ddd, J = 8.0, 5.2, 2.9 Hz, 1H), 6.99-6.95 (m, 2H), 6.72 (t, J = 4 Hz, 1H), 6.5-6.57 (m, 2H), 4.80 (d, J = 11.9 Hz, 1H), 4.37 (dd, J = 14.2, 6.8 Hz, 1H), 4.23 (dd, J = 14.3, 5.1 Hz, 1H), 4.17 (d, J = 12.2 Hz, 1H), 3.86- 3.84 (m, 1H), 3.72 (s, 3H), 3.73 − 3.58 (m, 1H), 2.73-3.69 (m, 1H), 2.66 − 2.49 (m, 1H), 2.00 (t, J = 2.7 Hz, 1H), 1.79 (s, 3H). LC-MS: 433.2[M + 1]+ |

### Prophetic Deuterated Analogs (PDAs) of Compounds of the Disclosure

### Example 138

The compounds provided in TABLE 2 are prophetic deuterated analogs (PDA) of Example 138. The Formula (A) is the generic formula of deuterated Example 138, wherein Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, and Y¹⁰ are each independently H or D. The deuterated analogs of Example 138 in TABLE 2 are predicted based on the metabolic profile of Example 138, with MetaSite (moldiscovery.com/software/metasite/). Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, and Y¹⁰ are most likely to be metabolized position based on MetaSite predictions.

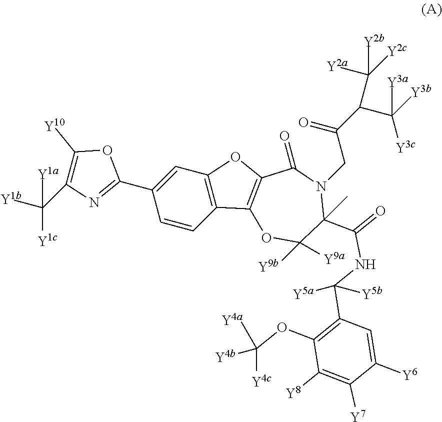

TABLE 2
Deuterated analogues of Formula (A)

| PDA # | Y^{1a}-Y^{1c} | Y^{2a}-Y^{2c} | Y^{3a}-Y^{3c} | Y^{4a}-Y^{4c} | Y^{5a}-Y^{5b} | Y⁶ | Y⁷ | Y⁸ | Y^{9a}-Y^{9b} | Y¹⁰ |
|---|---|---|---|---|---|---|---|---|---|---|
| A-1 | D | H | H | H | H | H | H | H | H | H |
| A-2 | H | D | H | H | H | H | H | H | H | H |
| A-3 | H | H | D | H | H | H | H | H | H | H |
| A-4 | H | H | H | D | H | H | H | H | H | H |
| A-5 | H | H | H | H | D | H | H | H | H | H |
| A-6 | H | H | H | H | H | D | H | H | H | H |
| A-7 | H | H | H | H | H | H | D | H | H | H |
| A-8 | H | H | H | H | H | H | H | D | H | H |
| A-9 | H | H | H | H | H | H | H | H | D | H |
| A-10 | H | H | H | H | H | H | H | H | H | D |
| A-11 | D | D | H | H | H | H | H | H | H | H |
| A-12 | D | H | D | H | H | H | H | H | H | H |
| A-13 | D | H | H | D | H | H | H | H | H | H |
| A-14 | D | H | H | H | D | H | H | H | H | H |
| A-15 | H | D | D | H | H | H | H | H | H | H |
| A-16 | H | D | H | D | H | H | H | H | H | H |
| A-17 | H | D | H | H | D | H | H | H | H | H |
| A-18 | H | H | D | D | H | H | H | H | H | H |
| A-19 | H | H | D | H | D | H | H | H | H | H |
| A-20 | H | H | H | D | D | H | H | H | H | H |

### Example 139

The compounds provided in TABLE 3 are prophetic deuterated analogs (PDA) of Example 139. The Formula (B) is the generic formula of deuterated Example 139, wherein Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹ and Y¹⁰ are each independently H or D. The deuterated analogs of Example 139 in TABLE 3 are predicted based on the metabolic profile of Example 139, with MetaSite (moldiscovery.com/software/metasite/). Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹ and Y¹⁰ are most likely to be metabolized position based on MetaSite predictions.

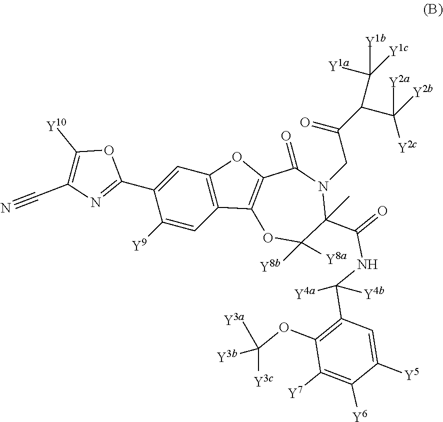

TABLE 3
Deuterated analogues of Formula (B)

| PDA # | Y^{1a}-Y^{1c} | Y^{2a}-Y^{2c} | Y^{3a}-Y^{3c} | Y^{4a}-Y^{4b} | Y⁵ | Y⁶ | Y⁷ | Y^{8a}-Y^{8b} | Y⁹ | Y¹⁰ |
|---|---|---|---|---|---|---|---|---|---|---|
| B-1 | D | H | H | H | H | H | H | H | H | H |
| B-2 | H | D | H | H | H | H | H | H | H | H |
| B-3 | H | H | D | H | H | H | H | H | H | H |
| B-4 | H | H | H | D | H | H | H | H | H | H |
| B-5 | H | H | H | H | D | H | H | H | H | H |
| B-6 | H | H | H | H | H | D | H | H | H | H |
| B-7 | H | H | H | H | H | H | D | H | H | H |
| B-8 | H | H | H | H | H | H | H | D | H | H |
| B-9 | H | H | H | H | H | H | H | H | D | H |
| B-10 | H | H | H | H | H | H | H | H | H | D |
| B-11 | D | D | H | H | H | H | H | H | H | H |
| B-12 | D | H | D | H | H | H | H | H | H | H |
| B-13 | D | H | H | D | H | H | H | H | H | H |
| B-14 | D | H | H | H | D | H | H | H | H | H |
| B-15 | H | D | D | H | H | H | H | H | H | H |
| B-16 | H | D | H | D | H | H | H | H | H | H |
| B-17 | H | D | H | H | D | H | H | H | H | H |
| B-18 | H | H | D | D | H | H | H | H | H | H |
| B-19 | H | H | D | H | D | H | H | H | H | H |
| B-20 | H | H | H | D | D | H | H | H | H | H |

### Example 140

The compounds provided in TABLE 4 are prophetic deuterated analogs (PDA) of Example 140. The Formula (C) is the generic formula of deuterated Example 140, wherein Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}Y^{8b}, Y⁹ and Y¹⁰ are each independently H or D. The deuterated analogs of Example 140 in TABLE 4 are predicted based on the metabolic profile of Example 140, with MetaSite (moldiscovery.com/software/metasite/). Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹ and Y¹⁰ are most likely to be metabolized position based on MetaSite predictions.

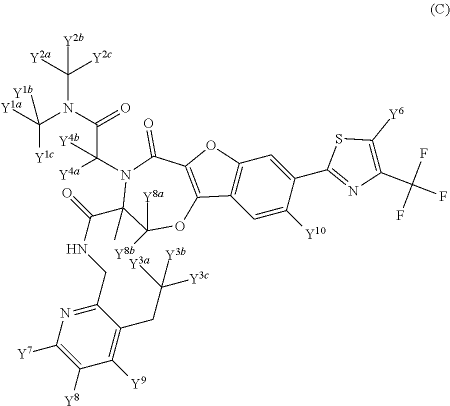

TABLE 4
Deuterated analogues of Formula (C)

| PDA # | Y^{1a}-Y^{1c} | Y^{2a}-Y^{2c} | Y^{3a}-Y^{3c} | Y^{4a}-Y^{4b} | Y⁵ | Y⁶ | Y⁷ | Y^{8a}-Y^{8b} | Y⁹ | Y¹⁰ |
|---|---|---|---|---|---|---|---|---|---|---|
| C-1 | D | H | H | H | H | H | H | H | H | H |
| C-2 | H | D | H | H | H | H | H | H | H | H |
| C-3 | H | H | D | H | H | H | H | H | H | H |
| C-4 | H | H | H | D | H | H | H | H | H | H |
| C-5 | H | H | H | H | D | H | H | H | H | H |
| C-6 | H | H | H | H | H | D | H | H | H | H |
| C-7 | H | H | H | H | H | H | D | H | H | H |
| C-8 | H | H | H | H | H | H | H | D | H | H |
| C-9 | H | H | H | H | H | H | H | H | D | H |
| C-10 | H | H | H | H | H | H | H | H | H | D |
| C-11 | D | D | D | H | H | H | H | H | H | H |
| C-12 | D | H | D | H | H | H | H | H | H | H |
| C-13 | D | H | H | D | H | H | H | H | H | H |
| C-14 | D | H | H | H | D | H | H | H | H | H |
| C-15 | H | D | D | H | H | H | H | H | H | H |
| C-16 | H | D | H | D | H | H | H | H | H | H |
| C-17 | H | D | H | H | D | H | H | H | H | H |
| C-18 | H | H | D | D | H | H | H | H | H | H |
| C-19 | H | H | D | H | D | H | H | H | H | H |
| C-20 | H | H | H | D | D | H | H | H | H | H |

### Example 141

The compounds provided in TABLE 5 are prophetic deuterated analogs (PDA) of Example 141. The Formula (0) is the generic formula of deuterated Example 141, wherein Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹ and Y¹⁰ are each independently H or D. The deuterated analogs of Example 141 in TABLE 5 are predicted based on the metabolic profile of Example 141, with MetaSite (moldiscovery.com/software/metasite/). Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹ and Y¹⁰ are most likely to be metabolized position based on MetaSite predictions.

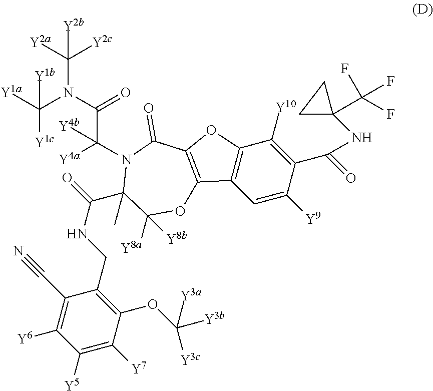

TABLE 5
Deuterated analogues of Formula (D)

| PDA # | Y^{1a}-Y^{1c} | Y^{2a}-Y^{2c} | Y^{3a}-Y^{3c} | Y^{4a}-Y^{4b} | Y⁵ | Y⁶ | Y⁷ | Y^{8a}-Y^{8b} | Y⁹ | Y¹⁰ |
|---|---|---|---|---|---|---|---|---|---|---|
| D-1 | D | H | H | H | H | H | H | H | H | H |
| D-2 | H | D | H | H | H | H | H | H | H | H |
| D-3 | H | H | D | H | H | H | H | H | H | H |
| D-4 | H | H | H | D | H | H | H | H | H | H |
| D-5 | H | H | H | H | D | H | H | H | H | H |
| D-6 | H | H | H | H | H | D | H | H | H | H |
| D-7 | H | H | H | H | H | H | D | H | H | H |
| D-8 | H | H | H | H | H | H | H | D | H | H |
| D-9 | H | H | H | H | H | H | H | H | D | H |
| D-10 | H | H | H | H | H | H | H | H | H | D |
| D-11 | D | D | H | H | H | H | H | H | H | H |
| D-12 | D | H | D | H | H | H | H | H | H | H |
| D-13 | D | H | H | D | H | H | H | H | H | H |
| D-14 | D | H | H | H | D | H | H | H | H | H |
| D-15 | H | D | D | H | H | H | H | H | H | H |
| D-16 | H | D | H | D | H | H | H | H | H | H |
| D-17 | H | D | H | H | D | H | H | H | H | H |
| D-18 | H | H | D | D | H | H | H | H | H | H |
| D-19 | H | H | D | H | D | H | H | H | H | H |
| D-20 | H | H | H | D | D | H | H | H | H | H |

### Example 142

The compounds provided in TABLE 6 are prophetic deuterated analogs (PDA) of Example 142. The Formula (E) is the generic formula of deuterated Example 142, wherein Y^{1a}, Y^{1b}, Y^{1c}, Y^{2a}, Y^{2b}, Y³, Y^{4a}, Y⁴, Y^{5a}, Y^{5b}, Y^{6a}, Y^{6b}, Y⁷, Y⁸, Y⁹ and Y¹⁰ are each independently H or D. The deuterated analogs of Example 142 in TABLE 6 are predicted based on the metabolic profile of 142, with MetaSite (moldiscovery.com/software/metasite/). Y^{1a}, Y^{1b}, Y^{1c}, Y^{2a}, Y^{2b}, Y³, Y^{4a}, Y⁴, Y^{5a}, Y^{5b}, Y^{6a}, Y^{6b}, Y⁷, Y⁸, Y⁹ and Y¹⁰ are most likely to be metabolized position based on MetaSite predictions.

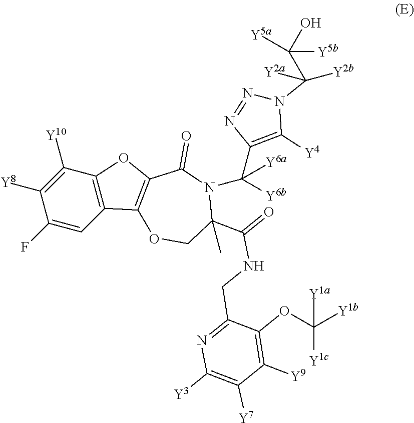

TABLE 6
Deuterated analogues of Formula (E)

| PDA # | Y^{1a}-Y^{1c} | Y^{2a}-Y^{2b} | Y³ | Y⁴ | Y^{5a}-Y^{5b} | Y^{6a}-Y^{6b} | Y⁷ | Y⁸ | Y^{s} | Y¹⁰ |
|---|---|---|---|---|---|---|---|---|---|---|
| E-1 | D | H | H | H | H | H | H | H | H | H |
| E-2 | H | D | H | H | H | H | H | H | H | H |
| E-3 | H | H | D | H | H | H | H | H | H | H |
| E-4 | H | H | H | D | H | H | H | H | H | H |
| E-5 | H | H | H | H | D | H | H | H | H | H |
| E-6 | H | H | H | H | H | D | H | H | H | H |
| E-7 | H | H | H | H | H | H | D | H | H | H |
| E-8 | H | H | H | H | H | H | H | D | H | H |
| E-9 | H | H | H | H | H | H | H | H | D | H |
| E-10 | H | H | H | H | H | H | H | H | H | D |
| E-11 | D | D | H | H | H | H | H | H | H | H |
| E-12 | D | H | D | H | H | H | H | H | H | H |
| E-13 | D | H | H | D | H | H | H | H | H | H |
| E-14 | D | H | H | H | D | H | H | H | H | H |
| E-15 | H | D | D | H | H | H | H | H | H | H |
| E-16 | H | D | H | D | H | H | H | H | H | H |
| E-17 | H | D | H | H | D | H | H | H | H | H |
| E-18 | H | H | D | D | H | H | H | H | H | H |
| E-19 | H | H | D | H | D | H | H | H | H | H |
| E-20 | H | H | H | D | D | H | H | H | H | H |

### Conditions for KLF2 Induction Cell Based Bioassay

In endothelial cell culture media, a stock of the highest working concentration was made at no more than 1% DMSO vehicle. The working stock was then serially diluted 1:2 to make an 11-point concentration response curve. Included in the 96-well plate were 2 reference compounds with known potencies. A DMSO column was included in the 96-well plate at the highest percent vehicle of the working stocks. The 96-well cell culture plate of primary human endothelial cells expressing luciferase under the control of the KLF2 promoter was dosed with 200 μL per well of the dilution series as well as the DMSO control column. The assay plate was incubated at 37° C. for 24 hours. The supernatant was collected at 24 hours.

20 μL of the supernatant was then placed in a 384-well plate with optical duplicates. The corners of the plate received 20 μL of the Gaussia Luciferase Enzyme positive control, as well as 20 μL of the 50 μM Coelenterizine substrate. The plate read is then calibrated using the control, and read using a Molecular Devices SpectraMax iD5, providing luminescence in relative light units (RLU). The plate reader injected 20 μL of the 50 μM Coelenterazine substrate immediately before taking the RLU measurement for each well.

The sample signals were then normalized to the average signal of the DMSO column, as well as to the maximum signal of one of the reference compounds, as shown in the equations below:

$$\frac{Mean of sample optical replicates ( RLU )}{Mean of all DMSO controls ( RLU )} = Vehicle Normalization$$

$$\frac{Vehicle Normalization - 1}{Reference Compound Maximum - 1} = Scaled to Maximum Signal$$

The EC₅₀ of each compound is then defined as when the concentration response curve passes 0.5. TABLE 7 shows the EC₅₀ values determined for the example compounds disclosed herein.

TABLE 7

| | GLuc KLF2 Conc | | GLuc KLF2 Conc |
|---|---|---|---|
| | Response (EC₅₀) | qPCR KLF2 Conc | Response (IC₅₀) |
| Ex# | Ave | Response (EC₅₀) | Avg |
| 1 | 0.04 | 0.01 | 0.22 |
| 2 | 0.02 | 0.01 | 0.20 |
| 3 | 0.06 | 0.01 | 0.34 |
| 4 | — | 0.00 | |
| 5 | 0.04 | 0.03 | 0.15 |
| 6 | 0.02 | 0.01 | 0.07 |

### Embodiments

The following non-limiting embodiments provide illustrative examples of the disclosure, but do not limit the scope of the disclosure.

Embodiment 1. A compound of Formula Ia:

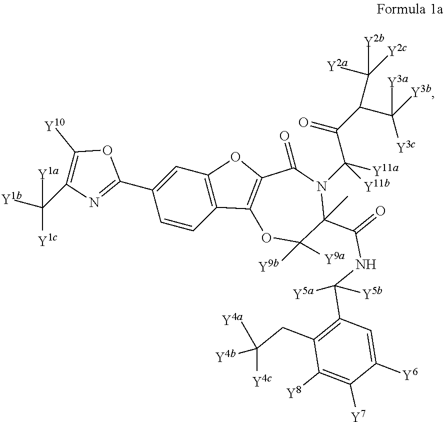

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H or D.

Embodiment 2. The compound of embodiment 1, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H.

Embodiment 3. The compound of embodiment 1, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H.

Embodiment 4. The compound of embodiment 1, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H.

Embodiment 5. The compound of embodiment 1, or a pharmaceutically acceptable salt thereof, wherein Y^{4a}, Y^{4b}, and Y^{4c} is each independently D, and Y^{1a}, Y^{1b}, Y^{1c}, Y^{2a}, Y^{2b}, Y^{2c}, Y^{3a}, Y^{3b}, Y^{3c} Y^{5a}, Y^{5b}, Y⁶, Y⁷, Y⁸, Y^{9a}, Y^{9b}, Y¹⁰, Y^{11a}, and Y^{11b} is each independently H.

Embodiment 6. The compound of embodiment 1, or a pharmaceutically acceptable salt thereof, wherein Y^{5a} and Y^{5b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y⁶, Y⁷, Y⁸, Y^{9a}, Y^{9b}, Y¹⁰, Y^{11a}, and Y^{11b} is each independently H.

Embodiment 7. The compound of embodiment 1, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁷, Y⁸, Y^{9a}, Y^{9b}, Y¹⁰, Y^{11a}, and Y^{11b} is each independently H.

Embodiment 8. The compound of embodiment 1, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁸, Y^{9a}, Y^{9b}, Y¹⁰, Y^{11a}, and Y^{11b} is each independently H.

Embodiment 9. The compound of embodiment 1, or a pharmaceutically acceptable salt thereof, wherein Y⁸ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y^{9a}, Y^{9b}, Y¹⁰, Y^{11a}, and Y^{11b} is each independently H.

Embodiment 10. The compound of embodiment 1, or a pharmaceutically acceptable salt thereof, wherein Y^{9a} and Y^{9b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y¹⁰, Y^{11a}, and Y^{11b} is each independently H.

Embodiment 11. The compound of embodiment 1, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y^{11a}, and Y^{11b} is each independently H.

Embodiment 12. The compound of embodiment 1, or a pharmaceutically acceptable salt thereof, wherein Y^{11a} and Y^{11b} are each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, and Y¹⁰ are each independently H.

Embodiment 13. A compound of Formula Ib:

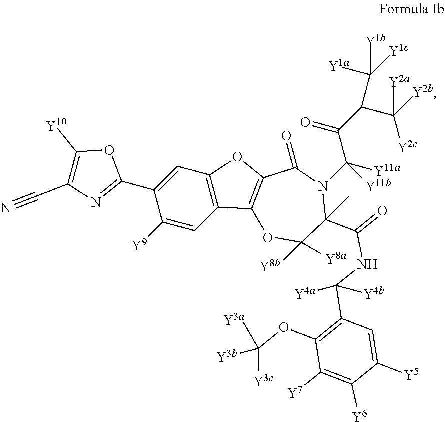

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

Embodiment 14. The compound of embodiment 13, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H.

Embodiment 15. The compound of embodiment 13, or a pharmaceutically acceptable salt thereof, wherein Y^{2a}, Y^{2b} and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H.

Embodiment 16. The compound of embodiment 13, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, Y¹⁰, Y^{11a}, and Y^{11b} is each independently H.

Embodiment 17. The compound of embodiment 13, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, Y¹⁰, Y^{11a}, and Y^{11b} is each independently H.

Embodiment 18. The compound of embodiment 13, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, Y¹⁰, Y^{11a}, and Y^{11b} is each independently H.

Embodiment 19. The compound of embodiment 13, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, Y¹⁰, Y^{11a}, and Y^{11b} is each independently H.

Embodiment 20. The compound of embodiment 13, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y^{8a}, Y^{8b}, Y⁹, Y¹⁰, Y^{11a}, and Y^{11b} is each independently H.

Embodiment 21. The compound of embodiment 13, or a pharmaceutically acceptable salt thereof, wherein Y^{5a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y⁹, Y¹⁰, Y^{11a}, and Y^{11b} is each independently H.

Embodiment 22. The compound of embodiment 13, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y^{11a}, and Y^{11b} is each independently H.

Embodiment 23. The compound of embodiment 13, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y^{11a}, and Y^{11b} is each independently H.

Embodiment 24. The compound of embodiment 13, or a pharmaceutically acceptable salt thereof, wherein Y^{11a} and Y^{11b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H.

Embodiment 25. A compound of Formula Ic:

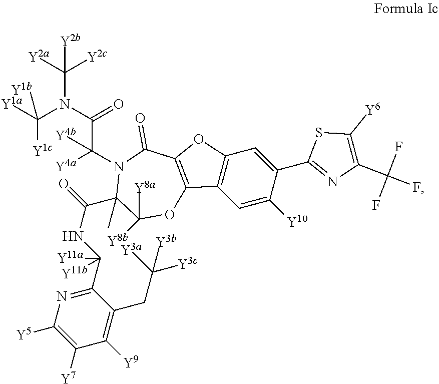

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

Embodiment 26. The compound of embodiment 25, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H.

Embodiment 27. The compound of embodiment 25, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰ is each independently H.

Embodiment 28. The compound of embodiment 25, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, Y¹⁰ is each independently H.

Embodiment 29. The compound of embodiment 25, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, Y¹⁰ is each independently H.

Embodiment 30. The compound of embodiment 25, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, Y¹⁰ is each independently H.

Embodiment 31. The compound of embodiment 25, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁷, Y^{8a}, Y^{8b}, Y⁹, Y¹⁰ is each independently H.

Embodiment 32. The compound of embodiment 25, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y^{8a}, Y^{8b}, Y⁹, Y¹⁰ is each independently H.

Embodiment 33. The compound of embodiment 25, or a pharmaceutically acceptable salt thereof, wherein Y^{8a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y, Y⁹, Y¹⁰ is each independently H.

Embodiment 34. The compound of embodiment 25, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y¹⁰ is each independently H.

Embodiment 35. The compound of embodiment 25, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, and Y⁹ is each independently H.

Embodiment 36. A compound of Formula Id:

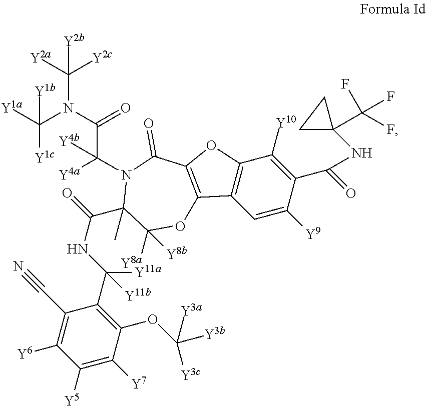

or a pharmaceutically acceptable salt thereof, wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

Embodiment 37. The compound of embodiment 36, or a pharmaceutically acceptable salt thereof, wherein Y^{1a}, Y^{1b}, and Y^{1c} is each independently D, and Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H.

Embodiment 38. The compound of embodiment 36, or a pharmaceutically acceptable salt thereof, wherein Y^{2a}, Y^{2b}, and Y^{2c} is each independently D, and Y^{1a}, Y^{1b}, Y^{1c}, Y^{3a}, Y^{3b}, Y^{3c}, Y^{4a}, Y^{4b}, Y⁵, Y⁶, Y⁷, Y^{8a}, Y^{8b}, Y⁹, and Y¹⁰ is each independently H.

Embodiment 39. The compound of embodiment 36, or a pharmaceutically acceptable salt thereof, wherein Y^{3a}, Y^{3b}, and Y^{3c} is each independently D, and Y^{1a}, Y^{1b}, Y^{1c}, Y^{2a}, Y^{2b}, Y^{2c}, Y^{4a}, Y^{4b}, Y⁵, Y⁶, Y⁷, Y^{8a}, Y^{8b}, Y⁹, and Y¹⁰ is each independently H.

Embodiment 40. The compound of embodiment 36, or a pharmaceutically acceptable salt thereof, wherein Y^{4a} and Y^{4b} is each independently D, and Y^{1a}, Y^{1b}, Y^{1c}, Y^{2a}, Y^{2b}, Y^{2c}, Y^{3a}, Y^{3b}, Y^{3c}, Y⁵, Y⁶, Y⁷, Y^{8a}, Y^{8b}, Y⁹, and Y¹⁰ is each independently H.

Embodiment 41. The compound of embodiment 36, or a pharmaceutically acceptable salt thereof, wherein Y⁵ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁶, Y⁷, Y^{8a}, Y^{8b}, Y⁹, and Y¹⁰ is each independently H.

Embodiment 42. The compound of embodiment 36, or a pharmaceutically acceptable salt thereof, wherein Y⁶ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁷, Y^{8a}, Y^{8b}, Y⁹, and Y¹⁰ is each independently H.

Embodiment 43. The compound of embodiment 36, or a pharmaceutically acceptable salt thereof, wherein Y⁷ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y^{8a}, Y^{8b}, Y⁹, and Y¹⁰ is each independently H.

Embodiment 44. The compound of embodiment 36, or a pharmaceutically acceptable salt thereof, wherein Y^{8a} and Y^{8b} is each independently D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y⁹, and Y¹⁰ is each independently H.

Embodiment 45. The compound of embodiment 36, or a pharmaceutically acceptable salt thereof, wherein Y⁹ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a} Y^{8b}, and Y¹⁰ is each independently H.

Embodiment 46. The compound of embodiment 36, or a pharmaceutically acceptable salt thereof, wherein Y¹⁰ is D, and Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, and Y⁹ is each independently H.

Embodiment 47. A compound of Formula Ie:

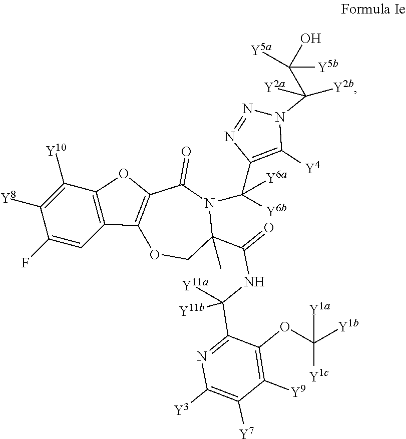

or a pharmaceutically acceptable salt thereof, wherein each Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H or D.

Embodiment 48. The compound of embodiment 47, or a pharmaceutically acceptable salt thereof, wherein Y^{1a}, Y^{1b}, and Y^{1c} is each independently D, and Y^{2a}, Y^{2b}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H.

Embodiment 49. The compound of embodiment 47, or a pharmaceutically acceptable salt thereof, wherein Y^{2a} and Y^{2b} is each independently D, and Y^{1a}, Y^{1b}, Y^{1c}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H.

Embodiment 50. The compound of embodiment 47, or a pharmaceutically acceptable salt thereof, wherein Y³ is D, and Y^{1a}, Y^{1b}, Y^{1c}, Y^{2a}, Y^{2b}, Y⁴, Y^{5a}, Y^{5b}, Y^{6a}, Y^{6b}, Y⁷, Y⁸, Y⁹, Y¹⁰, Y^{11a}, and Y^{11b} is each independently H.

Embodiment 51. The compound of embodiment 47, or a pharmaceutically acceptable salt thereof, wherein Y⁴ is D, and Y^{1a}, Y^{1b}, Y^{1c}, Y^{2a}, Y^{2b}, Y³, Y^{5a}, Y^{5b}, Y^{6a}, Y^{6b}, Y⁷, Y⁸, Y⁹, Y¹⁰, Y^{11a}, and Y^{11b} is each independently H.

Embodiment 52. The compound of embodiment 47, or a pharmaceutically acceptable salt thereof, wherein Y^{5a} and Y^{5b} is each independently D, and Y^{1a}, Y^{1b}, Y^{1c}, Y^{2a}, Y^{2b}, Y³, Y⁴, Y^{6a}, Y^{6b}, Y⁷, Y⁸, Y⁹, Y¹⁰, Y^{11a}, and Y^{11b} is each independently H.

Embodiment 53. The compound of embodiment 47, or a pharmaceutically acceptable salt thereof, wherein Y^{6a} and Y^{6b} is each independently D, and Y^{1a}, Y^{1b}, Y^{1c}, Y^{2a}, Y^{2b}, Y³, Y⁴, Y^{5a}, Y^{5b}, Y⁷, Y⁸, Y⁹, Y¹⁰, Y^{11a}, and Y^{11b} is each independently H.

Embodiment 54. The compound of embodiment 47, or a pharmaceutically acceptable salt thereof, wherein Y⁷ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁸, Y⁹, Y¹⁰, Y^{11a}, and Y^{11b} is each independently H.

Embodiment 55. The compound of embodiment 47, or a pharmaceutically acceptable salt thereof, wherein Y⁸ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁹, Y¹⁰, Y^{11a}, and Y^{11b} is each independently H.

Embodiment 56. The compound of embodiment 47, or a pharmaceutically acceptable salt thereof, wherein Y⁹ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y¹⁰, Y^{11a}, and Y^{11b} is each independently H.

Embodiment 57. The compound of embodiment 47, or a pharmaceutically acceptable salt thereof, wherein Y¹⁰ is D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, Y^{11a}, and Y^{11b} is each independently H.

Embodiment 58. The compound of embodiment 47, or a pharmaceutically acceptable salt thereof, wherein Y^{11a} and Y^{11b} is each independently D, and Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, and Y¹⁰ is each independently H.

Embodiment 59. The compound of any one of embodiments 1-58, or a pharmaceutically acceptable salt thereof, wherein the level of deuterium enrichment denoted D in the at least one position is at least about 50%.

Embodiment 60. The compound of any one of embodiments 1-58, or a pharmaceutically acceptable salt thereof, wherein the level of deuterium enrichment denoted D in the at least one position is at least about 70%.

Embodiment 61. The compound of any one of embodiments 1-58, or a pharmaceutically acceptable salt thereof, wherein the level of deuterium enrichment denoted D in the at least one position is at least about 80%.

Embodiment 62. The compound of any one of embodiments 1-58, or a pharmaceutically acceptable salt thereof, wherein the level of deuterium enrichment denoted D in the at least one position is at least about 90%.

Embodiment 63. The compound of any one of embodiments 1-58, or a pharmaceutically acceptable salt thereof, wherein the level of deuterium enrichment denoted D in the at least one position is at least about 98%.

Embodiment 64. A pharmaceutical composition comprising a compound of any one of embodiments 1-58, or a pharmaceutically acceptable salt thereof, and a pharmaceutically acceptable carrier.

Embodiment 65. A method of treating a condition comprising administering a therapeutically effective amount of a compound of any one of embodiments 1-58, or a pharmaceutically acceptable salt thereof, or the composition of embodiment 61, to a subject in need thereof, wherein the condition is an inflammatory disease or endothelial dysfunction.

Embodiment 66. The method of embodiment 62, wherein the condition is selected from the group consisting of atherosclerosis, coronary artery disease, stroke, peripheral arterial disease, coronary microvascular diseases, angina, systemic hypertension, pulmonary arterial hypertension, heart failure, and diabetic microvascular diseases, such as diabetic nephropathy, diabetic retinopathy or diabetic neuropathy, an autoimmune disease, inflammatory disease, infectious diseases, arterial and venous thrombosis, long covid, ischemia with non-obstructive coronary arteries (INOCA), antiphospholipid syndrome (APS), and chronic kidney disease.

Embodiment 67. The method of embodiment 63, wherein the condition is atherosclerosis.

Embodiment 68. The method of embodiment 63, wherein the condition is coronary artery disease.

Embodiment 69. The method of embodiment 63, wherein the administering is oral.

Embodiment 70. The method of embodiment 63, wherein the administering is intravenous.

Embodiment 71. The method of embodiment 63, wherein the administering is once a day.

Embodiment 72. The method of embodiment 63, wherein the administering is twice a day.

Embodiment 73. The method of embodiment 63, wherein the administering is three times a day.

Each of the embodiments described herein may be combined with any other embodiment(s) described herein not inconsistent with the embodiment(s) with which it is combined. In addition, any of the compounds described in the Examples, or pharmaceutically acceptable salts thereof, may be claimed individually or grouped together with one or more other compounds of the Examples, or pharmaceutically acceptable salts thereof, for any of the embodiment(s) described herein. Furthermore, each of the embodiments described herein envisions within its scope pharmaceutically acceptable salts of the compounds described herein.

It will be apparent to those skilled in the art that various modifications and variations may be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All references cited herein, including patents, patent applications, papers, textbooks, and the like, and the references cited therein, to the extent that they are not already, are hereby incorporated by reference in their entireties. In the event that one or more of the incorporated literature and similar materials differs from or contradicts this application, including but not limited to defined terms, term usage, described techniques, or the like, this application controls.

What is claimed is:

1. A compound of Formula Ia: 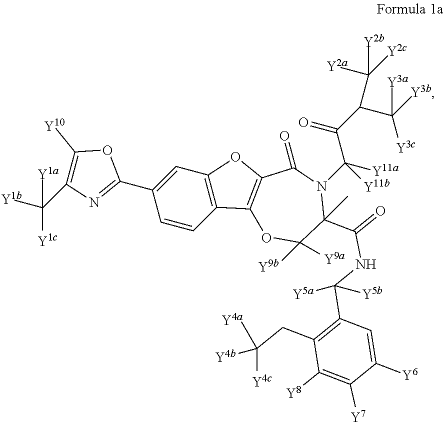
or a pharmaceutically acceptable salt thereof,
wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y^{4c}, Y^{5a}, Y^{5b}, Y⁶, Y⁷, Y⁸, Y^{9a}, Y^{9b}, Y¹⁰, Y^{11a}, and Y^{11b} is each independently H or D.

2. A compound of Formula Ib: 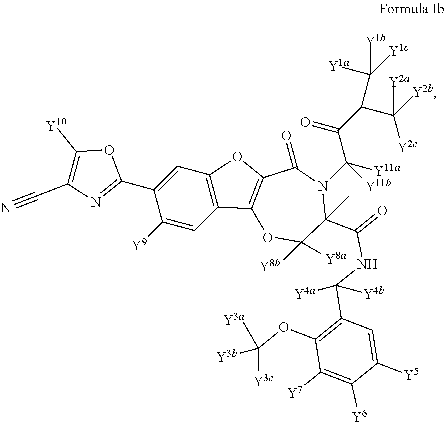
or a pharmaceutically acceptable salt thereof,
wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a} and Y^{11b} is each independently H or D.

3. A compound of Formula Ic: 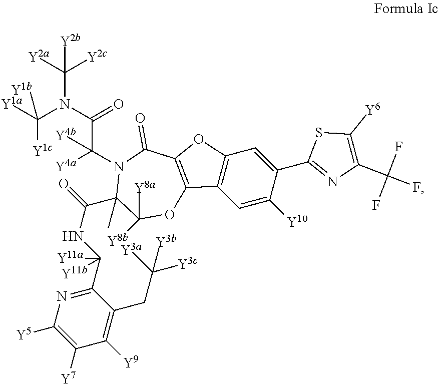
or a pharmaceutically acceptable salt thereof,
wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a}, and Y^{11b} is each independently H or D.

4. A compound of Formula Id: 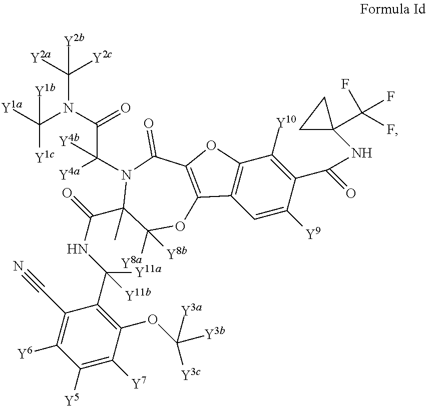
or a pharmaceutically acceptable salt thereof,
wherein each Y^{1a}, Y^{1b}, Y^{1c}, Y^{2a}, Y^{2b}, Y^{2c}, Y^{3a}, Y^{3b}, Y^{3c}, Y^{4a}, Y^{4b}, Y⁵, Y⁶, Y⁷, Y^{8a}, Y^{8b}, Y⁹, Y¹⁰, Y^{11a} and Y^{11b} is each independently H or D.

5. A compound of Formula Ie: 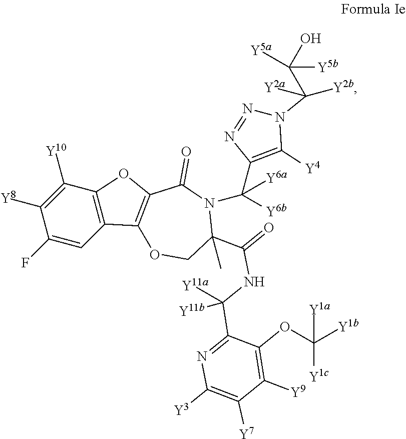
or a pharmaceutically acceptable salt thereof,
wherein each Y^{1a}, Y^{1b}, Y^{1c}, Y^{2a}, Y^{2b}, Y³, Y⁴, Y^{5a}, Y^{5b}, Y^{6a}, Y^{6b}, Y⁷, Y⁸, Y⁹, Y¹⁰, Y^{11a}, and Y^{11b} is each independently H or D.

6. The compound of claim 1, or a pharmaceutically acceptable salt thereof, wherein the level of deuterium enrichment denoted D in the at least one position is at least about 50%.

7. The compound of claim 1, or a pharmaceutically acceptable salt thereof, wherein the level of deuterium enrichment denoted D in the at least one position is at least about 70%.

8. The compound of claim 1, or a pharmaceutically acceptable salt thereof, wherein the level of deuterium enrichment denoted D in the at least one position is at least about 80%.

9. The compound of claim 1, or a pharmaceutically acceptable salt thereof, wherein the level of deuterium enrichment denoted D in the at least one position is at least about 90%.

10. The compound of claim 1, or a pharmaceutically acceptable salt thereof, wherein the level of deuterium enrichment denoted D in the at least one position is at least about 98%.

11. A pharmaceutical composition comprising a compound of claim 1, or a pharmaceutically acceptable salt thereof, and a pharmaceutically acceptable carrier.

12. A method of treating a condition comprising administering a therapeutically effective amount of a compound of claim 1, or a pharmaceutically acceptable salt thereof, or the composition of claim 61, to a subject in need thereof, wherein the condition is an inflammatory disease or endothelial dysfunction.

13. The method of claim 12, wherein the condition is selected from the group consisting of atherosclerosis, coronary artery disease, stroke, peripheral arterial disease, coronary microvascular diseases, angina, systemic hypertension, pulmonary arterial hypertension, heart failure, and diabetic microvascular diseases, such as diabetic nephropathy, diabetic retinopathy or diabetic neuropathy, an autoimmune disease, inflammatory disease, infectious diseases, arterial and venous thrombosis, long covid, ischemia with non-obstructive coronary arteries (INOCA), antiphospholipid syndrome (APS), and chronic kidney disease.

14. The method of claim 12, wherein the condition is atherosclerosis.

15. The method of claim 12, wherein the condition is coronary artery disease.

16. The method of claim 12, wherein the administering is oral.

17. The method of claim 12, wherein the administering is intravenous.
